SEQUENCE LISTING (74 sequences)

SEQ ID NO: 1 - DNA, 1218 bp, Zea mays
  source 1..1218: mol_type = unassigned DNA
atggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggataa

SEQ ID NO: 2 - protein, 405 aa, Zea mays
MDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAASTGLYHPYAQQPMRGGGWCKQEQDHAVIAAAHSLQDLHHLNLGAAGAHDFFSAGQQAAAAAMHGLGSIDSASLEHSTGSNSVVYNGGVGDSNGASAVGGSGGGYMMPMSAAGATTTSAMVSHEQVHARAYDEAKQAAQMGYESYLVNAENNGGG

SEQ ID NO: 3 - DNA, 1299 bp, Panicum virgatum
  source 1..1299: mol_type = unassigned DNA
cctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaaga

SEQ ID NO: 4 - DNA, 163 bp, Panicum virgatum
  source 1..163: mol_type = unassigned DNA
cgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagca

SEQ ID NO: 5 - DNA, 100 bp, Panicum virgatum
  source 1..100: mol_type = unassigned DNA
gccgcaggtcgtttggcgttccatgagcagacactgcagtgagtggatagcaatcaatagcgatgctctttggccttccatgaaacgcacgcactcatgt

SEQ ID NO: 6 - DNA, 1563 bp, Panicum virgatum
  source 1..1563: mol_type = unassigned DNA
cctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaagagccgcaggtcgtttggcgttccatgagcagacactgcagtgagtggatagcaatcaatagcgatgctctttggccttccatgaaacgcacgcactcatgtacgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagca

SEQ ID NO: 7 - DNA, 25312 bp, Synthetic construct
  misc_feature 1..25312: note = Plasmid
  source 1..25312: mol_type = other DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttacgctgtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagcgatttaaatctgcggttaccgaattcgagctcggtaccctgggatctgcccttactttaacgcctctaaccaacacccctttatctttataaggaacaataaacagaatttgccccactgttctaaatcacctaataatatccccagctaaaaacaataaaggtttcctagaattaagacaagcatgactgttcctccaggagggtttggaacattgttgcagtcttgcagatacgggcgaagggtgagaaacagagcggagggctggaggtgacctcggtagtcgacgccggagttgagcttgacaacgacggggcggcccctgatggacttgaggaagtccgatggcgtcttcaccgtcccgccggcgcccgaggcgggcctgtcgctgccgccgccgccgctgctcatcttgcgcgctgtgcccccggcggtgtccctgtgttgcggatcgcgggtgggccaggtggatgcgagggcgacccgtttggactccggccggagccgccggatccctggtcggtgtcagtgccgtttactctgggccccacgtgtcagtaccgtctgtagatgacaacaacccgtcgtccacagtcatgtccaaaatatcctttcttcttttttttcgattcggatatctatcttccttttttttttccaaaaatcttcttgacgcaccagcgcgcacgtttgtggtaaacgccgacacgtcggtcccacgtcgatagaccccacccaccagtgagtagcgtgtacgtattcgggggtgacggacgtgtcgccgtcgtcttgctagtcccattcccatctgagccacacatctctgaacaaaaaaaaggagggaggcctccacgcacatccccctccgtgccacccgccccaaaccctcgcgccgcctccgagacagccgccgcaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgagccccgcaagggtgctgacatcctcgtcgagtccctcgagcgctgcggcgtccgcgacgtaagtttctgcttctacctttgatatatatataataattatcattaattagtagtaatataatatttcaaatatttttttcaaaataaaagaatgtagtatatagcaattgcttttctgtagtttataacttcgtatagcatacattatacgaagttatacttttctaatatatgaccaaaatttgttaccgaattcaggcgacccatcgctgctttgtctacatcatgttcttcatcatcctccccaggcgacgcgtgctgctgttcttattcagactaccgttcgagtgactgcatggcgtacatctttctgcatcgactttgtacggctacatcgaacatatacacgagatgtctcgtgtgaatagagtcactaatgccttaagcatcggttactccgtagggtacattctgttcttcttatttgtgcatatttttattgttgtttactgattatacgagtagttatacatacatgcacatacatatcatcacatatatcacaatatttttctaaattaaattaaaactaaaaatgactaaatttctaacaccaacgacattgtaatgttttctccaacaactttacctattctacattgttctatttcgaatttcactctataaacaacatagtctacaatggaaaacagtgctttgtacgactatatacgcgatgtgtggctacaacataagacaatatagtcgtttgaagattgaacctatatatcggtacggttaatccgtctatgtacgtgggcatgacgaacacccgtgataacgaaggattaacgtgcacaatcataaatccaaagtaggagcggtgcatgatgagaatcgctctcagtactcgacataatgaaccttacgaggtacaacaggcaggcaggcagggaccaggggccgcctttatttcaggctcgctggccccacgggcgtgctgcgtgcacgaagggcactaccccaacctctcaccgaaaaccgcgctggatcggcaaatcaaacgaggtggtgccccgtgcccactctccacgtccacggcaccatccctctgcagccgctcaccagccatgccgtgtcgcggaacggcacaaccacccccaacccactcacgaaaccccgtcccggccgtgcccgtgtcggtccgcgctcggcaacgaggcggcccgcgctgctgagtcccctggacacccgacaccctgtcggccctttgtttattcatcccgaaatctcatctgcccccacggccgactgcgctgcgccgcccggatatatatacccatcgttatcgatcgactggggactctatcagtgatagagtctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactggtaccgttaacagatcattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgacccaccttgctagttggatattacctcttctcttcaaagtatccttgaacgctcaccggttatcaaatctctacactatagctctgtagtcttgctagatagttagttctttagctctcggtgaccaagcttggcgcgatcaagcttatcgataccgtcgacctcgaagcttggtcacccggtccgggcctagaaggccagcttcaagtttgtacaaaaaagcaggctccggccagaatggcccggaccgggttaccgaattcttaccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacctagtggtacctgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcaccgggatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctagatcgatgggccctggccgaagcttggtcacccggtccgggcctagaaggccagcttcaagtttgtaccctggagcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaaggatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatccagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaagctgttaccgaattcctgcccttaaggccaattgttcaagattcattcaacaattgaaacatctcccatgattaaatcagtataaggttgctatggtcttgcttgacaaagtttttttttgagggaatttcaactaaatttttgagtgaaactatcaaatactgattttaaaaaatttttataaaaggaagcgcagagataaaaggccatctatgctacaaaagtacccaaaaatgtaatcctaaagtatgaattgcattttttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcctcctgctctgctcctctccacaccacacggcacgaaaccgttacggcaccggcagcacccagcacgggagaggggattcctttcccaccgttccttccctttccgccccgccgctataaatagccagccccatccccagcttttttccccaatctcatctcctctctcctgttgttcggagcacacgcacaatccgatcgatccccaaatccccttcgtctctcctcgcgagcctcgtggatcccagcttcaaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggatatgtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcaggtcgactttaacttagcctaggatccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatagggtaccaatggccagttaacagatccagctgctgctgttctagggttcacaagtctgcctatttgtcttccccaatggagctatggttgtctggtctggtccttggtcgtgtcccgtttcattgtgtactatttacctgtaatgtgtatccttaagtctggtttgatggtgtctgaaacgttttgctgtggtagagcagcatggaagaactataatgaataagtgatccctaatcattgtgtccaaattttgcttctgctatacccttttgtgctgtttcttatgttttgcttaaaaatttgatctgacaaacaaatttgtctaaattatgctcttgttctgactgtgtactgtacacatttgtattgctaccggtttggcacatagttttctctttgattgcagatcaaacccttctgattgcagattgcttggctatggctgatgtgctcactgttcttctttagcttgtatattgctgatggtggcttggctacggctgaaaaagttgctgctgcgccgacttggcaactgttgctccagtaatgctttgtcctctcttcctagccgccgacttggcacttggcagctgttgctcctgtaatgcgttgttatctcttatctcttgagatgtactatgattttgaggctgtttgacttgtgccagattgccagtgctgatgctatattatctcaggtttttatgctagtatatgttttactgtgtgctgcctgtgtcgagttacgtaatctattgaaccgctatgcgtgttgatgccctgttgttgcttgccagattgatgcccctgttgcttgcagcttgcaagctgtagtgaaatcctaaaattcgaaaccaaatctattctgaacttttgaagatctgtgatgtccggaagaaaagattcaaacagataaacgggactctccagaatctataaaataaatgatcgacaaacgctttgctttggtaggcttcctagttagttagcggaccgaagcttcggtccgggcctagaaggccagcttcggccgccccatttcaaatatttttttcaaaataaaagaatgtagtatatagcaattgcttttctgtagtttataacttcgtatagcatacattatacgaagttatacttttctaatatatgaccaaaatttgttgatgtgcaggtcttcgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtggcgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagttggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactgatgactagctagtcagttaacagatctgccagatcctcggtgtacaaataacccgtcttatcctatgagacgggccggcgtcagtgtgttctggaggaatttttatgtcagagcctttttctttgtgtgcttgatgtagatgccaagggaagcttattggctgttgaagcttgatgcaaaataaattatggaactctgttttttgtttatctaataataactagcaaatatgcttccattgcattgaaactaacagccttttgtgtttccaagttttattttgtgacaatgtcatctatttcaattagttgtggaatcggaaacttgcaggactaacttggaaactccaatccctcagcatcctggactttttcctggtgtaatccatgtagatattattttaatcatcattttagttctggaggtttttccatctccggttttgctcccctttcttcaaaaaaaaaaaaaaaatgccgtaggcgccgcaacgcccacctgttgttcaaactcatgggcacgagtggctcgaagattttatacaacaattgctgtagtttcaccgttgctggtgaagaagcatttttttaaaaaaatatagtggtattcattttaattagtttagttgtgcagcgagcaaaatttggacatgctctgctcggaatctgatcgacctagacacagattagcagcagtagctttgtcatctgttccaagagttgcgatctgatagaagaaaaaaaaaccttcccttcaatgtaaaaccgaaactaacaagaaagaagcacagtgccgtttaggcaagtatggagtacgtattaagcatgtagaaggccatgcatgaaccactaacaagaaagaagcgcagtgccattcaggcaagcataagcatgtagattggcatgcatgaacaactaacagtagatcgtctctggtctgattagaagttttttgggaagccaagaaatcatgtacaactggttccatctcaaattccgtggcaaaaagaggcctaagcaacataaagctttccactggttgccatatatgatttgggcatttcactttgatcttaatgggccttgtaaagcccaagactcatgattatctttagttgatgctcttaattaggtgtgggcaaataattcaaactgtatgtacccgaccaaaaccaaagcaaaaataatcgaaccaaaccgaaaatttaaaaataaccgaatgaaaactaaatcctataactgaaagaactgaaaccgaatcaaaatatttaatgtaaccaaaaatatccgaaatataattatattgtcaaaaatattaataatttctagattaaataattaaaaatacttaaaaatttatataaaatagtaaaaatactcgaaaataaccacaaatattcaaaaacaaccgaaatatcccaaaatattcaaagcaaaataaccgaatggataccaaattttaaaaccgaaaaaactggaacaaaaccaaaatcgaaccaaaatttcaaaaatcgaataaatactaaactttagaacaaaaaaaaacgataaccgaatgtatacgaaccaaagccgaattagataaccgaacgtccaggactactcttaatctttccgccacttatgatttgggctattactttgtttataatgagccttttcaagctcaagttcatgattgtccgtgagatgagaaactgacttgttggattcgaaaccctagctagtattggttaatacttaatacataaatgacctgcattgacatcatcatccaagaaaataaaaattgtatgcttgagatatttagttttcctagctaggttttctttattttagtaccgaatctttaggtgtgccacgttaatttagacccattttttcatacttaccaactgagtctagtttaatcatgactataatcgtataaaatgattcagtcgacgtcattgcgaacgtatataaaatcatccaaattgacgtcattccaaagaggtaagcatgcttatctaagagtccgagcatactaaacaagacgacattttatttgcactccaaatcaaattttgtattgcctaaagaaaaacaatcaaactcaagtttcttaaaattaatttcattcaaactaatcactttcaatatctcacatattattcatgccatttctatttgtctaaacatgatttaaaaaaaaaagtaaaatacaaagattactatgcaaaaactctataaaaaaaaattcaaatttcttatttatttgtgacatcaaattttcaaaataatttttttaattatcggttgatccggtcagtcgataaaaacataaactttcagcgaccgttaaaactttcctactaccgatttagagaaaatcttagcttgaaacgtaattgtaacctgccttcatgcaagtcgcaagatatgtcatcctaagttgtatatgttttctcaaaagatgtatttacttgagaaaatacgtttcaacgttgatggacaaccaattaagaatcaagcacctttcgtaatcaatttaggcttatcgtctaaggtatactgatttacgacagttgactagacttataaggaacaaaataatagaatagtttctaaacacttcacctaatcatagagactaagatggtaattcgtaaacgacaaagcctagtgacactgtccattgtaaaattccacatcatattagtattaaacatatacatgtagtttcctgaacacatgtagtatcaaacacacttcgtggcttcttcctcgaaacctgcggccgctcattaattaagtcatctagttgaaggatcgctatcaactttgtatagaaaagttgggccgaattcgagctcggtacggccagaatggcccggaccgaagcttcctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaagagccgcaggtcgtttggcgttccatgagcagacactgcagtgagtggatagcaatcaatagcgatgctctttggccttccatgaaacgcacgcactcatgtacgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagcacctaggatccatggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggataaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccagcttcaagtttgtacaaaaaagcaggctccggccagaatggcccggaccgggttaccgaattcgagctcggtaccctgggatccgatatcgatgggccctggccgaagcttggtcacccggtccgggcctagaaggccgatctcccgggcacccagctttcttgtacaaagtggccgttaacggatcggccagaatggcccggaccgggttaccgaattcgagctcggtaccctgggatccgatatcgatgggccctggccgaagcttggtcacccggtccgggcctagaaggccagcttcggccgccccgggcaactttattatacaaagttgatagatcctcatgtcttcatcgtaagcttaaacaggccagaatggcctctggattcagcggcctagaaggccgaagtacttggtcttcctagtcgagtagatcacattcaagcttggtcacccggtccgtcaatattctgattccgatgacttcgtaggttcctagctcaagccgctcgtgtccaagcgtcacttacgattagctaatgattacggcatctaggaccgactagtggcgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 8 - DNA, 1215 bp, Zea mays
  source 1..1215: mol_type = unassigned DNA
atggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcgga

SEQ ID NO: 9 - protein, 405 aa, Zea mays; identical to SEQ ID NO: 2

SEQ ID NO: 10 - DNA, 45 bp, Synthetic construct
  misc_feature 1..45: note = Linker
  source 1..45: mol_type = other DNA
ggtggaggcggctcgggtggcggaggctccggaggcggtggctcg

SEQ ID NO: 11 - protein, 15 aa, Synthetic construct
  REGION 1..15: note = Linker
GGGGSGGGGSGGGGS

SEQ ID NO: 12 - DNA, 297 bp, Arabidopsis thaliana
  source 1..297: mol_type = unassigned DNA
acgtgcgccaaggacatccaaaaagcagccgcggaggcggcactcgcctttcaggacgagacctgcgacaccaccaccaccaaccacggcctcgacatggaggagaccatggtcgaagccatctacacccccgagcagagcgagggcgccttctacatggacgaagagacaatgttcggcatgccaaccctcctcgacaacatggccgaaggcatgctgctgccgcccccgagcgtccagtggaaccacaactacgacggcgaaggcgacggggatgtgtccctgtggagctactag

SEQ ID NO: 13 - protein, 98 aa, Arabidopsis thaliana
TCAKDIQKAAAEAALAFQDETCDTTTTNHGLDMEETMVEAIYTPEQSEGAFYMDEETMFGMPTLLDNMAEGMLLPPPSVQWNHNYDGEGDGDVSLWSY

SEQ ID NO: 14 - DNA, 1557 bp, Synthetic construct
  misc_feature 1..1557: note = DNA
  source 1..1557: mol_type = other DNA
atggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaggtggaggcggctcgggtggcggaggctccggaggcggtggctcgacgtgcgccaaggacatccaaaaagcagccgcggaggcggcactcgcctttcaggacgagacctgcgacaccaccaccaccaaccacggcctcgacatggaggagaccatggtcgaagccatctacacccccgagcagagcgagggcgccttctacatggacgaagagacaatgttcggcatgccaaccctcctcgacaacatggccgaaggcatgctgctgccgcccccgagcgtccagtggaaccacaactacgacggcgaaggcgacggggatgtgtccctgtggagctactag

SEQ ID NO: 15 - protein, 518 aa, Synthetic construct
  REGION 1..518: note = Protein
MDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAASTGLYHPYAQQPMRGGGWCKQEQDHAVIAAAHSLQDLHHLNLGAAGAHDFFSAGQQAAAAAMHGLGSIDSASLEHSTGSNSVVYNGGVGDSNGASAVGGSGGGYMMPMSAAGATTTSAMVSHEQVHARAYDEAKQAAQMGYESYLVNAENNGGGGGGGSGGGGSGGGGSTCAKDIQKAAAEAALAFQDETCDTTTTNHGLDMEETMVEAIYTPEQSEGAFYMDEETMFGMPTLLDNMAEGMLLPPPSVQWNHNYDGEGDGDVSLWSY

SEQ ID NO: 16 - DNA, 25651 bp, Synthetic construct
  misc_feature 1..25651: note = Plasmid
  source 1..25651: mol_type = other DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttacgctgtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagcgatttaaatctgcggttaccgaattcgagctcggtaccctgggatctgcccttactttaacgcctctaaccaacacccctttatctttataaggaacaataaacagaatttgccccactgttctaaatcacctaataatatccccagctaaaaacaataaaggtttcctagaattaagacaagcatgactgttcctccaggagggtttggaacattgttgcagtcttgcagatacgggcgaagggtgagaaacagagcggagggctggaggtgacctcggtagtcgacgccggagttgagcttgacaacgacggggcggcccctgatggacttgaggaagtccgatggcgtcttcaccgtcccgccggcgcccgaggcgggcctgtcgctgccgccgccgccgctgctcatcttgcgcgctgtgcccccggcggtgtccctgtgttgcggatcgcgggtgggccaggtggatgcgagggcgacccgtttggactccggccggagccgccggatccctggtcggtgtcagtgccgtttactctgggccccacgtgtcagtaccgtctgtagatgacaacaacccgtcgtccacagtcatgtccaaaatatcctttcttcttttttttcgattcggatatctatcttccttttttttttccaaaaatcttcttgacgcaccagcgcgcacgtttgtggtaaacgccgacacgtcggtcccacgtcgatagaccccacccaccagtgagtagcgtgtacgtattcgggggtgacggacgtgtcgccgtcgtcttgctagtcccattcccatctgagccacacatctctgaacaaaaaaaaggagggaggcctccacgcacatccccctccgtgccacccgccccaaaccctcgcgccgcctccgagacagccgccgcaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgagccccgcaagggtgctgacatcctcgtcgagtccctcgagcgctgcggcgtccgcgacgtaagtttctgcttctacctttgatatatatataataattatcattaattagtagtaatataatatttcaaatatttttttcaaaataaaagaatgtagtatatagcaattgcttttctgtagtttataacttcgtatagcatacattatacgaagttatacttttctaatatatgaccaaaatttgttaccgaattcaggcgacccatcgctgctttgtctacatcatgttcttcatcatcctccccaggcgacgcgtgctgctgttcttattcagactaccgttcgagtgactgcatggcgtacatctttctgcatcgactttgtacggctacatcgaacatatacacgagatgtctcgtgtgaatagagtcactaatgccttaagcatcggttactccgtagggtacattctgttcttcttatttgtgcatatttttattgttgtttactgattatacgagtagttatacatacatgcacatacatatcatcacatatatcacaatatttttctaaattaaattaaaactaaaaatgactaaatttctaacaccaacgacattgtaatgttttctccaacaactttacctattctacattgttctatttcgaatttcactctataaacaacatagtctacaatggaaaacagtgctttgtacgactatatacgcgatgtgtggctacaacataagacaatatagtcgtttgaagattgaacctatatatcggtacggttaatccgtctatgtacgtgggcatgacgaacacccgtgataacgaaggattaacgtgcacaatcataaatccaaagtaggagcggtgcatgatgagaatcgctctcagtactcgacataatgaaccttacgaggtacaacaggcaggcaggcagggaccaggggccgcctttatttcaggctcgctggccccacgggcgtgctgcgtgcacgaagggcactaccccaacctctcaccgaaaaccgcgctggatcggcaaatcaaacgaggtggtgccccgtgcccactctccacgtccacggcaccatccctctgcagccgctcaccagccatgccgtgtcgcggaacggcacaaccacccccaacccactcacgaaaccccgtcccggccgtgcccgtgtcggtccgcgctcggcaacgaggcggcccgcgctgctgagtcccctggacacccgacaccctgtcggccctttgtttattcatcccgaaatctcatctgcccccacggccgactgcgctgcgccgcccggatatatatacccatcgttatcgatcgactggggactctatcagtgatagagtctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactggtaccgttaacagatcattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgacccaccttgctagttggatattacctcttctcttcaaagtatccttgaacgctcaccggttatcaaatctctacactatagctctgtagtcttgctagatagttagttctttagctctcggtgaccaagcttggcgcgatcaagcttatcgataccgtcgacctcgaagcttggtcacccggtccgggcctagaaggccagcttcaagtttgtacaaaaaagcaggctccggccagaatggcccggaccgggttaccgaattcttaccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacctagtggtacctgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcaccgggatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcctaacttgacgactgcgtaactgactagagtcacacttagctgaccctagtcacttacaactgacttcgtgtcctagatcgatgggccctggccgaagcttggtcacccggtccgggcctagaaggccagcttcaagtttgtaccctggagcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaaggatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatccagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaagctgttaccgaattcctgcccttaaggccaattgttcaagattcattcaacaattgaaacatctcccatgattaaatcagtataaggttgctatggtcttgcttgacaaagtttttttttgagggaatttcaactaaatttttgagtgaaactatcaaatactgattttaaaaaatttttataaaaggaagcgcagagataaaaggccatctatgctacaaaagtacccaaaaatgtaatcctaaagtatgaattgcattttttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcctcctgctctgctcctctccacaccacacggcacgaaaccgttacggcaccggcagcacccagcacgggagaggggattcctttcccaccgttccttccctttccgccccgccgctataaatagccagccccatccccagcttttttccccaatctcatctcctctctcctgttgttcggagcacacgcacaatccgatcgatccccaaatccccttcgtctctcctcgcgagcctcgtggatcccagcttcaaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggatatgtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcaggtcgactttaacttagcctaggatccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatagggtaccaatggccagttaacagatccagctgctgctgttctagggttcacaagtctgcctatttgtcttccccaatggagctatggttgtctggtctggtccttggtcgtgtcccgtttcattgtgtactatttacctgtaatgtgtatccttaagtctggtttgatggtgtctgaaacgttttgctgtggtagagcagcatggaagaactataatgaataagtgatccctaatcattgtgtccaaattttgcttctgctatacccttttgtgctgtttcttatgttttgcttaaaaatttgatctgacaaacaaatttgtctaaattatgctcttgttctgactgtgtactgtacacatttgtattgctaccggtttggcacatagttttctctttgattgcagatcaaacccttctgattgcagattgcttggctatggctgatgtgctcactgttcttctttagcttgtatattgctgatggtggcttggctacggctgaaaaagttgctgctgcgccgacttggcaactgttgctccagtaatgctttgtcctctcttcctagccgccgacttggcacttggcagctgttgctcctgtaatgcgttgttatctcttatctcttgagatgtactatgattttgaggctgtttgacttgtgccagattgccagtgctgatgctatattatctcaggtttttatgctagtatatgttttactgtgtgctgcctgtgtcgagttacgtaatctattgaaccgctatgcgtgttgatgccctgttgttgcttgccagattgatgcccctgttgcttgcagcttgcaagctgtagtgaaatcctaaaattcgaaaccaaatctattctgaacttttgaagatctgtgatgtccggaagaaaagattcaaacagataaacgggactctccagaatctataaaataaatgatcgacaaacgctttgctttggtaggcttcctagttagttagcggaccgaagcttcggtccgggcctagaaggccagcttcggccgccccatttcaaatatttttttcaaaataaaagaatgtagtatatagcaattgcttttctgtagtttataacttcgtatagcatacattatacgaagttatacttttctaatatatgaccaaaatttgttgatgtgcaggtcttcgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtggcgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagttggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactgatgactagctagtcagttaacagatctgccagatcctcggtgtacaaataacccgtcttatcctatgagacgggccggcgtcagtgtgttctggaggaatttttatgtcagagcctttttctttgtgtgcttgatgtagatgccaagggaagcttattggctgttgaagcttgatgcaaaataaattatggaactctgttttttgtttatctaataataactagcaaatatgcttccattgcattgaaactaacagccttttgtgtttccaagttttattttgtgacaatgtcatctatttcaattagttgtggaatcggaaacttgcaggactaacttggaaactccaatccctcagcatcctggactttttcctggtgtaatccatgtagatattattttaatcatcattttagttctggaggtttttccatctccggttttgctcccctttcttcaaaaaaaaaaaaaaaatgccgtaggcgccgcaacgcccacctgttgttcaaactcatgggcacgagtggctcgaagattttatacaacaattgctgtagtttcaccgttgctggtgaagaagcatttttttaaaaaaatatagtggtattcattttaattagtttagttgtgcagcgagcaaaatttggacatgctctgctcggaatctgatcgacctagacacagattagcagcagtagctttgtcatctgttccaagagttgcgatctgatagaagaaaaaaaaaccttcccttcaatgtaaaaccgaaactaacaagaaagaagcacagtgccgtttaggcaagtatggagtacgtattaagcatgtagaaggccatgcatgaaccactaacaagaaagaagcgcagtgccattcaggcaagcataagcatgtagattggcatgcatgaacaactaacagtagatcgtctctggtctgattagaagttttttgggaagccaagaaatcatgtacaactggttccatctcaaattccgtggcaaaaagaggcctaagcaacataaagctttccactggttgccatatatgatttgggcatttcactttgatcttaatgggccttgtaaagcccaagactcatgattatctttagttgatgctcttaattaggtgtgggcaaataattcaaactgtatgtacccgaccaaaaccaaagcaaaaataatcgaaccaaaccgaaaatttaaaaataaccgaatgaaaactaaatcctataactgaaagaactgaaaccgaatcaaaatatttaatgtaaccaaaaatatccgaaatataattatattgtcaaaaatattaataatttctagattaaataattaaaaatacttaaaaatttatataaaatagtaaaaatactcgaaaataaccacaaatattcaaaaacaaccgaaatatcccaaaatattcaaagcaaaataaccgaatggataccaaattttaaaaccgaaaaaactggaacaaaaccaaaatcgaaccaaaatttcaaaaatcgaataaatactaaactttagaacaaaaaaaaacgataaccgaatgtatacgaaccaaagccgaattagataaccgaacgtccaggactactcttaatctttccgccacttatgatttgggctattactttgtttataatgagccttttcaagctcaagttcatgattgtccgtgagatgagaaactgacttgttggattcgaaaccctagctagtattggttaatacttaatacataaatgacctgcattgacatcatcatccaagaaaataaaaattgtatgcttgagatatttagttttcctagctaggttttctttattttagtaccgaatctttaggtgtgccacgttaatttagacccattttttcatacttaccaactgagtctagtttaatcatgactataatcgtataaaatgattcagtcgacgtcattgcgaacgtatataaaatcatccaaattgacgtcattccaaagaggtaagcatgcttatctaagagtccgagcatactaaacaagacgacattttatttgcactccaaatcaaattttgtattgcctaaagaaaaacaatcaaactcaagtttcttaaaattaatttcattcaaactaatcactttcaatatctcacatattattcatgccatttctatttgtctaaacatgatttaaaaaaaaaagtaaaatacaaagattactatgcaaaaactctataaaaaaaaattcaaatttcttatttatttgtgacatcaaattttcaaaataatttttttaattatcggttgatccggtcagtcgataaaaacataaactttcagcgaccgttaaaactttcctactaccgatttagagaaaatcttagcttgaaacgtaattgtaacctgccttcatgcaagtcgcaagatatgtcatcctaagttgtatatgttttctcaaaagatgtatttacttgagaaaatacgtttcaacgttgatggacaaccaattaagaatcaagcacctttcgtaatcaatttaggcttatcgtctaaggtatactgatttacgacagttgactagacttataaggaacaaaataatagaatagtttctaaacacttcacctaatcatagagactaagatggtaattcgtaaacgacaaagcctagtgacactgtccattgtaaaattccacatcatattagtattaaacatatacatgtagtttcctgaacacatgtagtatcaaacacacttcgtggcttcttcctcgaaacctgcggccgctcattaattaagtcatctagttgaaggatcgctatcaactttgtatagaaaagttgggccgaattcgagctcggtacggccagaatggcccggaccgaagcttcctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaagagccgcaggtcgtttggcgttccatgagcagacactgcagtgagtggatagcaatcaatagcgatgctctttggccttccatgaaacgcacgcactcatgtacgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagcacctaggatccatggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaggtggaggcggctcgggtggcggaggctccggaggcggtggctcgacgtgcgccaaggacatccaaaaagcagccgcggaggcggcactcgcctttcaggacgagacctgcgacaccaccaccaccaaccacggcctcgacatggaggagaccatggtcgaagccatctacacccccgagcagagcgagggcgccttctacatggacgaagagacaatgttcggcatgccaaccctcctcgacaacatggccgaaggcatgctgctgccgcccccgagcgtccagtggaaccacaactacgacggcgaaggcgacggggatgtgtccctgtggagctactagggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccagcttcaagtttgtacaaaaaagcaggctccggccagaatggcccggaccgggttaccgaattcgagctcggtaccctgggatccgatatcgatgggccctggccgaagcttggtcacccggtccgggcctagaaggccgatctcccgggcacccagctttcttgtacaaagtggccgttaacggatcggccagaatggcccggaccgggttaccgaattcgagctcggtaccctgggatccgatatcgatgggccctggccgaagcttggtcacccggtccgggcctagaaggccagcttcggccgccccgggcaactttattatacaaagttgatagatcctcatgtcttcatcgtaagcttaaacaggccagaatggcctctggattcagcggcctagaaggccgaagtacttggtcttcctagtcgagtagatcacattcaagcttggtcacccggtccgtcaatattctgattccgatgacttcgtaggttcctagctcaagccgctcgtgtccaagcgtcacttacgattagctaatgattacggcatctaggaccgactagtggcgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 17 - DNA, 1991 bp, Zea mays
  source 1..1991: mol_type = unassigned DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 18 - DNA, 1062 bp, Zea mays
  source 1..1062: mol_type = unassigned DNA
ccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaac

SEQ ID NO: 19 - DNA, 8924 bp, Synthetic construct
  misc_feature 1..8924: note = Plasmid
  source 1..8924: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccacgcgtgaagagcggttaccagagctggtcacctttgtccaccaagatggaactgtcttcggccagaatggccatctggattcagcaggcctagaaggccatttatcgctatcaactttgtatagaaaagttgggccgagctcggtacggccagaatggcccggaccgggttaccaagcggccgcatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagcaacgagatcatgagccaatcaaagaggagtgatgtagacctaaagcaataatggagccatgacgtaagggcttacgcccatacgaaataattaaaggctgatgtgacctgtcggtctctcagaacctttactttttatgtttggcgtgtatttttaaatttccacggcaatgacgatgtgaccgtcgacccactaaaacattgctttgtcaaaagctaaaaaagatgatgcccgacagccacttgtgtgaagcatgagaagccggtccctccactaagaaaattagtgaagcatcttccagtggtccctccactcacagctcaatcagtgagcaacaggacgaaggaaatgacgtaagccatgacgtctaatcccagggcccggtgaccaatcgaattcttaccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaaacaacgtaagcgcttacgcaccgaacaacgtaagcgcttacgcaccgaacaacgtaagcgcttacgcacccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagaacagatcattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtcacccggtccgggcctagaaggccagcttcaagtttgtacaaaaaagcaggctccggccagaatggcccggaccgggttaccgaattcgagctcggtaccctgggatccgatatcgatgggccctggccgaagcttggtcacccggtccgggcctagaaggccgatctcccgggcacccagctttcttgtacaaagtggccgttaacggatcggccagaatggcccggaccgaagccgaggccggccacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccggcggatctcgactctagaggatcgctcaggaaggccgctgagatagagccatggggaggagggcgtgttgcgcgaaggaaggcgttaagagaggggcgtggacgagcaaggaggacgatgccttggccgcctacgtcaaggcccacggcgaaggcaaatggagggaagtgccccagaaagccggtttgcgtcggtgcggcaagagctgccggctgcggtggctgaactacctccggcccaacatcaggcgcggcaacatctcctacgacgaggaggatctcatcatccgcctccacaggctcctcggcaacaggtggtcgctgattgcaggcaggctgcctggccgaacagacaatgaaatcaagaactactggaacagcacgctgggccggagggcaggcgccggcgccggcgccggcggcagatccacgatggcgctttcagcttcccgagttcagcaggcggaagaactgctgcaacgacctgctgagaggcagctgatgaggagccagcttgctgcagccgccaggagcatcaactggagctacgccctcttctggtccatttcagacactcaaccaggggtgctgacgtggacggacgggttctacaacggcgaggtgaagacgcggaagatttccaactccgtggagctgacatccgaccagctcgtcatgcagaggagcgaccagctccgggagctctacgaggccctcctgtcgggcgagggcgaccgccgcgctgcgcctgcgcggccggccggctctctgtcgccggaggacctcggcgacaccgagtggtactacgtggtgtccatgacctacgccttccggccaggccaagggttgcccggcaggagtttcgcgagcgacgagcatgtctggctgtgcaacgcgcacctcgccggcagcaaagccttcccccgcgcgctcctcgccaagagcgcgtccattcagtcaatcctctgcatcccggttatgggcggcgtgcttgagcttggtacaactgacacggtgccggaggccccggacttggtcagccgagcaaccgcggctttctgggagccgcagtgcccgagctccagcccgtcaggacgagcaaacgagacgggcgaggccgcagcagacgacggcacgtttgcgttcgaggaactcgaccacaataatggcatggacgacatagaggcgatgaccgccgccgggggacacgggcaggaggaggagctaagactaagagaagccgaggccctgtcagacgacgcaagcctggagcacatcaccaaggagatcgaggagttctacagcctctgcgacgaaatggacctgcaggcgctaccactaccgctagaggacggctggaccgtggacgcgtccaatttcgaggtcccctgctcttccccgcagccagcgccgcctccggtggatagggctaccgctaacgtcgccgccgacgcctcaagggcacccgtctacggctctcgcgcgacgagtttcatggcttggacgaggtcctcgcagcagtcgtcgtgctccgacgacgcggcgcccgcagcagtagtgccggccatcgaggagccgcagagattgctgaagaaagtggtggccggcggcggtgcttgggagagctgtggcggcgcgacgggagcagcacaggaaatgagtggcactggcaccaagaaccacgtcatgtcggagcgaaagcgacgagagaagctcaacgagatgttcctcgtcctcaagtcactgcttccgtccattcacagggtgaacaaagcgtcgatcctcgccgaaacgatagcctacctcaaggagcttcagagaagggtgcaagagctggagtccagtagggaacctgcgtcgcgcccatccgaaacgacgacaaggctaataacaaggccctcccgtggcaataatgagagtgtgaggaaggaggtctgcgcgggctccaagaggaagagcccagagctcggcagagacgacgtggagcgccccccggtcctcacgatggacgccggcaccagcaacgtcaccgtcaccgtctcggacaaggatgtgctcctggaggtgcagtgccggtgggaggagctcctgatgacgcgagtgttcgacgccatcaagagcctccatttggacgtcctctcggttcaggcttcagcgccagatggcttcatggggcttaagatacgagctcagtttgctggctccggtgccgtcgtgccctggatgatcagcgaggctcttcgcaaagctatagggaacccctgggtcgtcgtcgcgccggacaccggctcgcacgccaccccggccgcgacgtcgggcgcctgcgagacgggccagaatagcgccgctcatcgcgcggaccccgactcagccgggacgacgacgacctcggcggcggcggtgtgggcgcccaaggccgtgcggtgcacgggcggactcttcttcttccaccgggacacgacgccggcgcacgcgggcgagacggcgacgccaatggccggtggaggtggaggaggaggaggagaagcagggtcgtcggacgactgcagctcggcggcgtcggtatcgcttcgcgtcggaagccacgacgagccgtgcttctccggcgacggtgacggcgactggatggacgacgtgagggccctggcgtcgtttctggagtccgacgaggactggctccgctgtcagacggccgggcagcttgcgtagtggccaggtaccactaactatctatactgtaataatgttgtatagccgccggatagctagctagtttagtcattcagcggcgatgggtaataataaagtgtcatccatccatcaccatgcgtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgcgtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttggcgcgccggtcacccggtccgggcctagaaggccagcttcggccgccccgggcaactttattatacaaagttgatagataaatcagatatctggttcctaaggacccggcggaccgattaaactgattcggtccgacgcgttacctaataacttcgtatagcatacattatacgaagttattagctaactaactagtaagcttaagccatgggtcgacggatccgttacctgagctgattccgatgacttcgtaggttcctagctcaagccgctcgtgtccaagcgtcacttacgattagctaatgattacggcatctaggaccgactagctaactaactagtacgtacgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 20 - DNA, 8344 bp, Synthetic construct
  misc_feature 1..8344: note = Plasmid
  source 1..8344: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccacgcgtgaagagcggttaccagagctggtcacctttgtccaccaagatggaactgtcttcggccagaatggccatctggattcagcaggcctagaaggccatttatcgctatcaactttgtatagaaaagttgggccgaattcgagctcggtacggccagaatggcccggaccgggttaccgaattcgagctcggtaccctgggatccgatatcgatgggccctggccgaagcttggtcacccggtccgggcctagaaggccagcttcaagtttgtacaaaaaagcaggctccggccagaatggcccggaccgaagcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaaggatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccgatctcccgggcacccagctttcttgtacaaagtggccgttaacggatcggccagaatggcccggaccgggttaccgaattcgagctcggtaccctgggatccgatatcgatgggccctggccgaagcttggtcacccggtccgggcctagaaggccagcttcggccgccccgggcaactttattatacaaagttgatagataaatcagatatctggttcctaaggacccggcggaccgattaaactgattcggtccgacgcgttacctaataacttcgtatagcatacattatacgaagttattagctaactaactagtaagcttcctagggtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaagcttaagccatgggtcgacggatccgttacctgagctgattccgatgacttcgtaggttcctagctcaagccgctcgtgtccaagcgtcacttacgattagctaatgattacggcatctaggaccgactagctaactaactagtacgtacgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 21 - DNA, 11889 bp, Synthetic construct
  misc_feature 1..11889: note = Plasmid
  source 1..11889: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccacgcgtgaagagcggttaccagagctggtcacctttgtccaccaagatggaactgtcttcggccagaatggccatctggattcagcaggcctagaaggccatttatcgctatcaactttgtatagaaaagttgggccgaattcgagctcggtacggccagaatggcccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggataaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccagcttcaagtttgtacaaaaaagcaggctccggccagaatggcccggaccgaagcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaaggatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccgatctcccgggcacccagctttcttgtacaaagtggccgttaacggatcggccagaatggcccggaccgggttaccgaattcgagctcggtaccctgggatccgatatcgatgggccctggccgaagcttggtcacccggtccgggcctagaaggccagcttcggccgccccgggcaactttattatacaaagttgatagataaatcagatatctggttcctaaggacccggcggaccgattaaactgattcggtccgacgcgttacctaataacttcgtatagcatacattatacgaagttattagctaactaactagtaagcttcctagggtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaagcttaagccatgggtcgacggatccgttacctgagctgattccgatgacttcgtaggttcctagctcaagccgctcgtgtccaagcgtcacttacgattagctaatgattacggcatctaggaccgactagctaactaactagtacgtacgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 22 - DNA, 11504 bp, Synthetic construct
  misc_feature 1..11504: note = Plasmid
  source 1..11504: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccacgcgtgaagagcggttaccagagctggtcacctttgtccaccaagatggaactgtcttcggccagaatggccatctggattcagcaggcctagaaggccatttatcgctatcaactttgtatagaaaagttgggccgaattcgagctcggtacggccagaatggcccggaccgggttaccgaattcttaccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaaccctaggatccatggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggataagtcgaggcgtacctagtggtacctgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcaccggtaccgagctcgaattccggtccgggcctagaaggccagcttcaagtttgtacaaaaaagcaggctccggccagaatggcccggaccgaagcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaaggatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccgatctcccgggcacccagctttcttgtacaaagtggccgttaacggatcggccagaatggcccggaccgggttaccgaattcgagctcggtaccctgggatccgatatcgatgggccctggccgaagcttggtcacccggtccgggcctagaaggccagcttcggccgccccgggcaactttattatacaaagttgatagataaatcagatatctggttcctaaggacccggcggaccgattaaactgattcggtccgacgcgttacctaataacttcgtatagcatacattatacgaagttattagctaactaactagtaagcttcctagggtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaagcttaagccatgggtcgacggatccgttacctgagctgattccgatgacttcgtaggttcctagctcaagccgctcgtgtccaagcgtcacttacgattagctaatgattacggcatctaggaccgactagctaactaactagtacgtacgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 23 - DNA, 20962 bp, Synthetic construct
  misc_feature 1..20962: note = Plasmid
  source 1..20962: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccacgcgtgaagagcggttaccagagctggtcacctttgtccaccaagatggaactgtcttcggccagaatggccatctggattcagcaggcctagaaggccatttatcgctatcaactttgtatagaaaagttgggccgaattcgagctcggtacggccagaatggcccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggcaccgaagaagaagcgcaaggtgatggacaagaagtacagcatcggcctcgacatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgaccacatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagacaagagaccacgggaccgccacgatggcgagctgggaggccgcaagcgggcaaggtaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgccccggggaattgggggtagaccggttgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgaaggggcttcggaggatgttttagagctagaaatagcaagttaaaataaggctagtccgttatcaacttgaaaaagtggcaccgagtcggtgctttttttttgcggccgcgaattctgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgaaggggcttcggaggatgttttagagctagaaatagcaagttaaaataaggctagtccgttatcaacttgaaaaagtggcaccgagtcggtgctttttttttgaattccaccggtgttaacggcccgggttaacggcaaacgctaattagtcactaggcccaggaaacactaactaagccctagcttatcctagcgcaacctagttaggtcttccccaagtttgtacaaaaaagcaggctccggccagaatggcccggaccgaagcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaaggatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccgatctcccgggcacccagctttcttgtacaaagtggccgttaacggatcggccagaatggcccggaccgggttaccgaattcgagctcggtaccctgggatccgatatcgatgggccctggccgaagcttggtcacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggataaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccagcttcggccgccccgggcaactttattatacaaagttgatagataaatcagatatctggttcctaaggacccggcggaccgattaaactgattcggtccgacgcgttacctaataacttcgtatagcatacattatacgaagttattagctaactaactagtaagcttcctagggtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaagcttaagccatgggtcgacggatccgttacctgagctgattccgatgacttcgtaggttcctagctcaagccgctcgtgtccaagcgtcacttacgattagctaatgattacggcatctaggaccgactagctaactaactagtacgtacgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 24 - DNA, 20502 bp, Synthetic construct
  misc_feature 1..20502: note = Plasmid
  source 1..20502: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccacgcgtgaagagcggttaccagagctggtcacctttgtccaccaagatggaactgtcttcggccagaatggccatctggattcagcaggcctagaaggccatttatcgctatcaactttgtatagaaaagttgggccgaattcgagctcggtacggccagaatggcccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggcaccgaagaagaagcgcaaggtgatggacaagaagtacagcatcggcctcgacatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgaccacatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagacaagagaccacgggaccgccacgatggcgagctgggaggccgcaagcgggcaaggtaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgccccggggaattgggggtagaccggttgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgaaggggcttcggaggatgttttagagctagaaatagcaagttaaaataaggctagtccgttatcaacttgaaaaagtggcaccgagtcggtgctttttttttgcggccgcgaattctgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgaaggggcttcggaggatgttttagagctagaaatagcaagttaaaataaggctagtccgttatcaacttgaaaaagtggcaccgagtcggtgctttttttttgaattccaccggtgttaacggcccgggttaacggcaaacgctaattagtcactaggcccaggaaacactaactaagccctagcttatcctagcgcaacctagttaggtcttccccaagtttgtacaaaaaagcaggctccggccagaatggcccggaccgaagcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaaggatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccgatctcccgggcacccagctttcttgtacaaagtggccgttaacggatcggccagaatggcccggaccgggttaccgaattcttaccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaaccctaggatccatggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggataagtcgaggcgtacctagtggtacctgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcaccggtaccgagctcgaattccggtccgggcctagaaggccagcttcggccgccccgggcaactttattatacaaagttgatagataaatcagatatctggttcctaaggacccggcggaccgattaaactgattcggtccgacgcgttacctaataacttcgtatagcatacattatacgaagttattagctaactaactagtaagcttcctagggtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaagcttaagccatgggtcgacggatccgttacctgagctgattccgatgacttcgtaggttcctagctcaagccgctcgtgtccaagcgtcacttacgattagctaatgattacggcatctaggaccgactagctaactaactagtacgtacgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 25 - DNA, 8082 bp, Synthetic construct
  misc_feature 1..8082: note = DNA
  source 1..8082: mol_type = other DNA
cgctttgctttgtcgcgacgcgagcctgcgcgttgcgttcgtggcgtcggggagggggggggatggcaagcgccggtttgctgtattggtcacgtgatgcggctgcgcaacggccggggccagggcggtgccgccgcggcgacgggatcgcgcgtggttggtgtgacggacgggtgggggcgcaccgagcgcatgccgcctccatgaggattgaggaggccctcccgtccggatgcatcacacatgccactggcttggctggctgggacgtgatcccctgcaccggctacgtgttcttccttctcaccgagaagatgctgacaatgaaaacggcggcctcgtcacggctccgacgcgatccgtgggcgggcgggcgcgtccctcgccggagctggcgccgcgtgtcgccttctctggaacgccacgtgcctgcgtgcgcgcgcgcgcgcgagacgtcctagtccccagtgatttcgcttggaaattttttttatccgaatcttgagggtctggaggagcttcatggctaccgagtgcccgtcagcccgtggtatttttaggtgtgaacggagcccggttctcgtcggatcgccagctttaataagcacgaggtagtaggtgcgaagaggagaacggccttaactaactgctctcctcacgctgccaggaatgagcttgacgacggtgctagctggcacgaccttattaattttttattactatattactagaaaacgattgatcacggcgaaaattaagcactcgcctcatgacccgatcctccgaagccccttcgcatcacggcgctttgcggtgcttgtctttgacttcttcttttttttaaaaaaaaactcgcaatgggtagtacacacgatagccgcagtctttaaaagttgattactcttctcaatctgacctgacgtttggctcaagtcgaactcgagccgggaaaaaataaatatattagttaacaactaaaccctcctgtaaattaagcctaagtacctaggatacgtccctcctgcctctaactacttttttattactatctttattatcgatagaatctagggtgtgtttgtttagcgggttgacgcgggttggaacagattcaacacataacaactcgtgtttggtttaaaaggctcgcgggttagattggttccgggtcgaaatacgtcttgaaaatatgggttctaaggattcctaaaaacaagcgaaccatttcgacccacttcctctctaccctactgtcacccatgtcttatccactcgagaccagcacagggcgtgccgccactgtcgagggaccccgccgacctaccctccgacgttaatcccctgcctgccactagtcgtcggtcccccactggcctacccgccagcatgctggcctccgccagcgccgtcagactccctgaccggcttgttgcgcctcccaccgtcagactcctcggctggcatgccctccgatgtcgtggtgttctccgataggcacaacttgcgagccttgctagtgctgtcgtcgaacaggtaaaggtcagtgctcgtcatcgcctagacaagcctgctagtcatgtcaaatgttaaaaattcactgaaatgtatcttagtgttgttgggatgttcctttaactataactgaaatgtaacagtgagacctcatcactaaaaaattatgacttggttaataatggctttaatttatgtatatattgtgcttattgatttctcaaatattaaaatagtgtcgtttcatttattttgaatgttgtcatcttattgaattgaattgaagatatatcattttgggtaatatgaccagtaaggttactatattaagtttatccctcatcatgaaataaataagagatggtgtagctactatgagttctatgtctatctagtgtccatgccggacatgtttcgttgcatgcttaatagtagcgccgagagcgttccctcctcttctcgcaagcgtgcctgccaactttgaaaaaaaaaatatcttagatatacctttgctctttgctttgagcgcttgtattttgtccttctgtttcttctttaccgagttaagtgcttaactagcatttgtggcaatcaccatcgatcatctgatctatggctgcatctttcttcaatcttggatgtggacttgacctagcccacgcacaacgacacctgcagagtaactgttagcccactaattatctactaccaaacgcaaggttttcaaaatctcccctgtatatacatatatggctttcaatcccgcaaatagatacgttcattcataggagtacgcacgtgtgccgtgcacgagggtctcggtttataccgtcttcttttttttttgaagaagaaaaaaaataaacttgtcctaagcaggacgtacggtagcacctactgccaataagatatatattgctgctggtaaaatattttataatgtttatgatcttatcattgaaaatttcgtgtgaagtttttaaatttaaaattcaaattttataaacggtcttggatgtagaaactattaaaataaaacttttagatctcaaaaagttatgcaactttatagttggtcacattttcaaatgaactcatttagtgccttaaataatcaaattactctcgatttgttatagtacatgggaaatggaaacgtaatataaatataattggtgtaatagtgtagtgctatagaagggtatgcgcgagagagaggttgcgagttcgaatctcaccatttacaaaacatataagtttgattcaaaataatagtgaaaaatgatagggcaacgggtaagggtagggttggagagttgttcctagaatttaaaaaatgttttgctgttttttttttaatttttttgattcttaatttgccgagtgtttttctttgccgagtgctttttgacactcggcaaagtctttgccgagtgtccgaaaaaaacactcggcaaagaaccctttgccgatgaaatctttgtcgagtgttaagcctttgtcgagtgtaaaatagtctttgccgagtgtcttagacactcggcaaagaacgtgattccggtagtgaatagatacgttcattcataggagtacgcacgtgtgccgtgcaggagggtctcggtttacaccatgttctttttttagaagaaaaaaattaaacttgtcctaagcaggacgtacggtagcacctactgccaataagatatatattgctgctggtaaaatattttataatgtttatggctttataatgtttaaacttgtcctaatagctgacacatgacagactctaacctttcagtttccttgaaaagcagtaggccagcagccacaccccaggcgacagatggcgcgatcgtccacatggtggaggtggatagcagcaatccaggcttttccagtttggttccaaggcggcctccgtgtttttaattttacacacacatatatacatcctcctctcgttttcttcggccacttcttttttcttcttcttcttctttttgttgcacacatatatgtattccagggaaatttcccttggtgtgtgccatatacatgcatgtggaggagacggggatggagagctgtgagctagtcccctttgtactacagaaagaaagtgcgtgctgcccttatttctggaatgaaaaggcagaatgggaagagggggaaaaaaggggatagatagatgtggaggttgagggagacccacaccaccatataaacccccctcttccaaactagtcacacatggcacaagcacacagctcatcagttcttcttcgtcgtcttggctagctgcctagctagctagctagttcctcactcctctccgagaaccccctttttcatctctctctctctctctcggtctcgcccagctcctttcgatgttatcgtctctgctggcctcgcaactcgtcgagagagactcgtgaaaaagaatccagatatagctagagagaccgagagggcagctagcatgtcgatgagcttcttgagcatggtggaggcggagctgccgccggggttccggttccacccgagggacgacgagctcatctgcgactacctcgcgcccaagctcggcgccaagcccggcttctccggctgccgcccgcccatggtcgacgtcgacctcaacaaggtcgagccatgggatctccccggtgagtacgtacgtccacgcaagtagctagataaatcttttttttaatttgctagcgctcctttctttttctacgaccttcttctcagctaataaaagctagctagctgtgcgtcttccttgattaattcctccatgatacttgcaccaagttgatgcatatatgcgcacggagaggagaaagatgcagagaaatcacacacgtcaaatttccctagcttgctcgacacaacacgatccttaaatctgtccctgtttattatattctagtccacgatcaaaactcacagctttttccgtttttttaaataaaacacagcctcttaatttctgctgttttttttaaaaaaatcatgattttccagaataaagctatttgggatcgatcctctgttgcttcagcgtcaagagaaacaccacacgagtccccggccaaatgtgctgttcatacactaaactgctgcacacacatcacaccactctcctacatacttttatttttcgtcagcaagctagctaggatattaatatcgacgttttgctacctaaacctttagaaataattcatgatcgatctcgttttcatgcctatatatgatcatttccttgtcttcctcttcactcggtatatgttttctatacctcttctttttgttaccatgaacttgacttgttgatgacgcctccccaacaaatgaatagttatgaccagctgtattttaagtcggaggtcaaactaaaattaagcagtaggtatacacacaccattaattaattgaaaaacacatcgatttaatttgttttccgttcgtcggctgtaattatccaatttgtacacgatggtttctttgtttgtttctgaaaccttctcgcgcgcgcggtggtataatatcaacgtcacgatcagagtacgcgtgtacacgtactgcgctctcgatatgcacaagaccacactaacaaagctagccaagcgtccgtcgtttggacgagctttttgccaatctaaaaagaagagtaaatttaatttgaatcatttggggtggttcatctaaattcaccgcgaggaacgacaaccctctgctatatggtatatctctcgatttgtttaagctggtgaagccgtcctgctgcctgccaaatagatagactctagcaactactgagttttaaaaagagaaacaaaattaaagaatgacaaccttttacggccaaaccccttttaacgtctggccaacgaatcttctcttcgacctgttttagcttgtgtccaaaatgctttccgtcgtagccctgcgttcgttactagataccgctcaccatttgatcttgattatgaaaacattgaccttatatatatatatatatatatatagccaagtattttttaatctaatatggaataagagaaaaactaatgtgcgtgcaactatcagaagcaaaattttctgtttttttcttctaacattgttgggtaaattagaattcaaaattttatgtatcggtatactatatattataataccctccagctcagattcaaatccgttttagataattaatagattcatacaacacttaatggtgtgtgtacatgttttatacacgtgtctagattcattatcatctatttgaatatagtagacataaaaatcaagatcaaaaccgaatactactttagcacggagtgagtaaaagtataagtataatattttttggggatatttgggatttttttaattttgattacacgacgattatattatattgtacggccggggaagcgttgattgtgcacagactgcatgccgtacaaactgcaagaataatcgtacatgtacatgcatgctgatgaactgaaagatcttgagctcgtggccctcctaatttgttaaataaaaatgcacatcggaaagcaacaaactgcaaatcacgccttacgcatgagatctcagattcccagcacgctctctctctctctctctgtctgcacatcacgtcacgtgtccctcccctcttgtgaattgatgcccggatcagcacactactggtagcagaaggtcccaaaactaagctatgcatgggcgggctacatacatacaccacccccatgctatttctagcagagtggctccaaacctcagcatccacagtacaggctacagcgtgtggttgtgatggtgaccacaagaatcctactacatgcatgctgatgatgtgctgcagtgaacactcactcactgcccatgaacagcgtctcctacctgccagccagtgccaagaacaatgaatgaatgattaactgctggcttcgcttgctgaaccattcccctcctcgtgtgcgcatgatgggcatgcagtggcggcgtcggtggggccgcgggagtggtacttcttcagcctcaaggaccgcaagtacgcgacggggcagcggacgaaccgggccacggtgtccgggtactggaaggcgacggggaaggaccgaccagtggtggcggcgcggcgaggcgcgctggtggggatgcgcaagacgctcgtgttctaccaggggagggcgcccaagggcaggaagacggagtgggtgatgcacgagtacaggatggagccagctgctcctcttcttgatcaccaaccctcctcatccaactccaaggtaaaatcggtcgatccattattcgatgaaacgacaagttaagactccattaattcgacgaactgaccgggggtgttttaactgtttcgttgtcgtccatcgatccttctcctgtcatcatgtcaccagcaggatgaagattgggtgctgtgcagagtcatctgcaagaagaaactggcagcaggaggccgcgcaggagggggcagctcgaggagcctggtcgccagcaacggcggccgcgagaccgcgccagccaccccgccgccgccgccgctgccacctcgcatggacacggacgccaccctagcacagctccaggccgccatgcacgccaccgccggcgcgctcgagcaggtgccctgcttctccagcttcaacaacaacactgccagctctagagctgctgccgcagcagcagcagcgcagccatgctacctgcccagcatggccacaggcggcagccacggcacgacgagctactacctagaccacgcgatgctgccgcctgagctgggtggctgcttcgatcctctccacggcgacaagaagctgctcaaggcggtgctgggccagctcggcggcgacgcggtggcgccgggcctgagcctgcagcacgagatggccgcgggcgctgtcgtcgcttcatccgcttggatgaatcacttctaggggacattagttcagcagcgaacctaagtatgtgattggttgcgtatattatgggaaatacatacatatatacataattgtgtggtgaaacatttgtgtgtgaggcaaggagtagagcatgcgtcatttttttggttgctgccgatcgatttggacgaaggccatgaggcaaggagtagaactctgtaatataatgatggctgctcaagtgtgtgtacacattcagatgcagaggtcgtatgtcatgagcgatgatcaggagaatttcagattggttattttactgtgcctgaactcaaccccaagatcacacttgacaccactcactcactcactcactcagagacacagtgcacattcatagaatcatcatggtaaacagacagaagacagaacagtggctgccaaacccaatcaatcatgccatttatgcaggaaagtaggcatgacagtcacatccgagcatctcttctttaccacaccatctttctttttccgatgtgtattgttgcgatgacaatcatgctaaccaagcagtcagctgagtgagggctcctgggcactggctttccactgcact

SEQ ID NO: 26 - DNA, 444 bp, Zea mays
  source 1..444: mol_type = unassigned DNA
atgacttgccttgtggccgtcaattctaattctgtaaatagtagtttcaacatcgagtgtggagatgctacagatgccaactttgagaaatacaatctcagtagaggctccgtagctggcattcttacacttgatacagtggcagactatcttccgagaaggggacccctgaagcagagaaggacaggtattgcgtatatagcaaacgtcgcggtgcggaaggaggagcgacggaaagggatcgcgaagatgttggtccaagaagcagaggctcgggcaaggagctggggatgccggtcgatggcactgcactgcgacgtaaacaacgtggccgcgctacgcctgtacagaaaccaaggcttcaagtgcatacgagtgccggaaggagctaaatggcccgaacccaagatagctaaaggagcggagtacaacttcatgatgaag

SEQ ID NO: 27 - DNA, 447 bp, Zea mays
  source 1..447: mol_type = unassigned DNA
atgaatgacggcgttgatgaacacatgatatggttggtagggttgaagaatatcttcgcccgacagcttcctaatatgcccaaagaatatattgtacgccttgtcatggatagaactcacaagtcaatgatggttatcaggaacaatattgtcgtggggggcattacttatcgcccttatgcaagccagagatttggagaaatagcgttttgtgctatcacagctgatgagcaagttaaaggctatggaacaagattaatgaatcatttgaaacaacatgcacgggatgctgatgggctcacacatttcttaacctatgctgataacaatgctgttggctattttgtaaagcagggtttcacaaaggagatcacattggacaaagaaagatggcaagggtacattaaagattatgacggaggaatattgatggagtgtaaaattgac

SEQ ID NO: 28 - DNA, 1155 bp, Zea mays
  source 1..1155: mol_type = unassigned DNA
atggaggtgggcacgcgcgtgatgtgccggtggcgcgaccagaagctccacccggtcaaggtgatcgagcgccgcaagggctccgcgtgctcttcccccgccgactacgagtattacgtccactacaccgagttcaatagaagacttgacgagtgggttaagcttgatcagcttgatcttgacactgtcgagaccgttgtcgatgagaaagtagaagataaggcaacaagcttaaagatgacacggcaccagaaacgtaaaattgatgaaacacatgtggagcaaggacatgaggagcttgatgcagccagcttgcgggaacatgaagaattcacaaaggtgaaaaatatcgcgaagatcgaactggggagatatgagatagacacatggtatttctctccttttccaccagagtataatgactgcccgaagctgtttttctgtgaattttgcctcaacttcatgaagcgcaaagaacagcttcagaggcacatgaagaagtgtgatcttaagcaccccccaggtgatgaaatctatagaagtggaactctatcaatgttcgaggttgatgggaagaagaacaaggtttatggacagaacctttgttatctggctaagctatttcttgatcacaagacattgtactatgatgttgatttgttcttgttttatgtcctctgcgaatgtgatgatcggggatgtcatatggtgggatatttctctaaggaaaaacactccgaagaatcttataatttggcttgcattctcactctccccccataccaaagaaaaggatatgggaagttcctgattgctttctcatatgaactttccaagaaagaaggtaaagttgggacaccggagcgccctctctctgatcttggtctgctcagctatagaggttattggactagagtgcttctggaaattctaaagaaacataagggaaacatatctataaaggaactaagcgacatgacggcaataaaagcagatgacatattgagcacgctacagagcctggacctaatccagtaccgcaaagggcagcacgtcatctgcgctgacccgaaggtcctcgaccgccacctcaaggcggcggggcggggcggcctggacgtcgacgttagcaagctcatatggactccctacaag

SEQ ID NO: 29 - DNA, 1371 bp, Zea mays
  source 1..1371: mol_type = unassigned DNA
atgatggcgctcaagcagaaggacaccgacgccgccgccaccgccaccggtaccaagaagcgccgccgcgtcttcttctccgacaccgatgctggtgttgaggcaaatgagtgtatgaaagtgtttctagtatggaatccaggcgaggtgagctctgtagactgcactgctattcagccatttgacctaaaccatttttttggtgaagatggaaaaatatatggttacaaaaatcttaagataaatgtgtggataagtgcgaaatctttccatggatatgctgatgtttcatttgacgaaacatctgatggagggaaaggaataacagatctaaagcctgttcttcagaacatttttggagaaaacttggtagaaaaagaggaattcctgcatacattttcgaaggaatgtgaatatatcagaactgcagtgacaaatggtagtgccattaagcatgatggctcatatgaatcagatcctgcagttgagattgttcgagttgaactccaaggtgctgctgcatttctttattctcgtctggtacctcttgttcttcttctggttgaaggttccacgcctatagatattggagaacatggatgggaaatgcttctggtagtaaagaaggcaacacaagaggctggttcaaaatttgaactgctaggttttgcagctgtgcataacttctatcattatcctgagagtattcgcttacgaatcagccagatacttgtgttgccaccttatcagggtgaaggccatggtcttggtcttcttgaagcaatcaattatattgcacagtctgagaacatttacgatgttacaatagaaagcccttctgattacctgcagtatgtgcgatcatccattgactgcctccgtttgctcatgtttgatccgatcaaacctgcactcggtgctattgtcttgtctctgaaggagaccaacctgtcaaaaagggcccaaagtttgagaatggttccaccagctgacctgatggagactgtccgtcagaagctgaagataaacaagaaacagttcctccggtgctgggaaattctgatcttcctaagcctcgactctcaggatcataagagcatggacaacttcagggcctgcatatatgaccgcatgaagggggagatccttggcagtgcctctggaactaacaggaagcgtctgttgcagatgccaaccagtttcaataaggaggcatcttttgctgtgtattggacacaggagattgaggatgaggatgaacaaactgttgagcagcagccagaggatctcaagacgcaggagcaacagctcaatgagctcgtggacatccaaatcgaggagattgctggggtcgccaagaacgtcacttca

SEQ ID NO: 30 - DNA, 177 bp, Zea mays
  source 1..177: mol_type = unassigned DNA
gtggcctttcaaatactaaatgaaaagacaactgtcttgtctcctgaagtgcttctttctgctggagttccttgctgcagattggttcaaaaccctggtgaatttgttatcacattccctggagcttatcattctggttttagccatggatttaattgtggagaagcaacaaacatt

SEQ ID NO: 31 - DNA, 180 bp, Synthetic construct
  misc_feature 1..180: note = DNA
  source 1..180: mol_type = other DNA
ccgaaaaagaaacgcaaagttgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgtta

SEQ ID NO: 32 - protein, 148 aa, Zea mays
MTCLVAVNSNSVNSSFNIECGDATDANFEKYNLSRGSVAGILTLDTVADYLPRRGPLKQRRTGIAYIANVAVRKEERRKGIAKMLVQEAEARARSWGCRSMALHCDVNNVAALRLYRNQGFKCIRVPEGAKWPEPKIAKGAEYNFMMK

SEQ ID NO: 33 - protein, 149 aa, Zea mays
MNDGVDEHMIWLVGLKNIFARQLPNMPKEYIVRLVMDRTHKSMMVIRNNIVVGGITYRPYASQRFGEIAFCAITADEQVKGYGTRLMNHLKQHARDADGLTHFLTYADNNAVGYFVKQGFTKEITLDKERWQGYIKDYDGGILMECKID

SEQ ID NO: 34 - protein, 385 aa, Zea mays
MEVGTRVMCRWRDQKLHPVKVIERRKGSACSSPADYEYYVHYTEFNRRLDEWVKLDQLDLDTVETVVDEKVEDKATSLKMTRHQKRKIDETHVEQGHEELDAASLREHEEFTKVKNIAKIELGRYEIDTWYFSPFPPEYNDCPKLFFCEFCLNFMKRKEQLQRHMKKCDLKHPPGDEIYRSGTLSMFEVDGKKNKVYGQNLCYLAKLFLDHKTLYYDVDLFLFYVLCECDDRGCHMVGYFSKEKHSEESYNLACILTLPPYQRKGYGKFLIAFSYELSKKEGKVGTPERPLSDLGLLSYRGYWTRVLLEILKKHKGNISIKELSDMTAIKADDILSTLQSLDLIQYRKGQHVICADPKVLDRHLKAAGRGGLDVDVSKLIWTPYK

SEQ ID NO: 35 - protein, 457 aa, Zea mays
MMALKQKDTDAAATATGTKKRRRVFFSDTDAGVEANECMKVFLVWNPGEVSSVDCTAIQPFDLNHFFGEDGKIYGYKNLKINVWISAKSFHGYADVSFDETSDGGKGITDLKPVLQNIFGENLVEKEEFLHTFSKECEYIRTAVTNGSAIKHDGSYESDPAVEIVRVELQGAAAFLYSRLVPLVLLLVEGSTPIDIGEHGWEMLLVVKKATQEAGSKFELLGFAAVHNFYHYPESIRLRISQILVLPPYQGEGHGLGLLEAINYIAQSENIYDVTIESPSDYLQYVRSSIDCLRLLMFDPIKPALGAIVLSLKETNLSKRAQSLRMVPPADLMETVRQKLKINKKQFLRCWEILIFLSLDSQDHKSMDNFRACIYDRMKGEILGSASGTNRKRLLQMPTSFNKEASFAVYWTQEIEDEDEQTVEQQPEDLKTQEQQLNELVDIQIEEIAGVAKNVTS

SEQ ID NO: 36 - protein, 59 aa, Zea mays
VAFQILNEKTTVLSPEVLLSAGVPCCRLVQNPGEFVITFPGAYHSGFSHGFNCGEATNI

SEQ ID NO: 37 - protein, 60 aa, Synthetic construct
  REGION 1..60: note = Protein
PKKKRKVGRADALDDFDLDMLGSDALDDFDLDMLGSDALDDFDLDMLGSDALDDFDLDML

SEQ ID NO: 38 - DNA, 48 bp, Zea mays
  source 1..48: mol_type = unassigned DNA
aaacagatcaacaactggtttatcaatcaaaggaagcggcactggaaa

SEQ ID NO: 39 - protein, 16 aa, Zea mays
KQINNWFINQRKRHWK

SEQ ID NO: 40 - DNA, 1311 bp, Synthetic construct
  misc_feature 1..1311: note = DNA
  source 1..1311: mol_type = other DNA
atggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaggtggaggcggctcgggtggcggaggctccggaggcggtggctcgaaacagatcaacaactggtttatcaatcaaaggaagcggcactggaaatag

SEQ ID NO: 41 - protein, 436 aa, Synthetic construct
  REGION 1..436: note = Protein
MDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAASTGLYHPYAQQPMRGGGWCKQEQDHAVIAAAHSLQDLHHLNLGAAGAHDFFSAGQQAAAAAMHGLGSIDSASLEHSTGSNSVVYNGGVGDSNGASAVGGSGGGYMMPMSAAGATTTSAMVSHEQVHARAYDEAKQAAQMGYESYLVNAENNGGGGGGGSGGGGSGGGGSKQINNWFINQRKRHWK

SEQ ID NO: 42 - DNA, 1621 bp, Zea mays
  source 1..1621: mol_type = unassigned DNA
ctacatctaatgcatagaaacaccaagatcacattgtactagcaaaatgtcatagaagactagttaaaaccttgtttggtccgctcaaccttaaaaatcacctaggagacatgctagaagtatctcaacagagaatgacaccatatagtagtggcaccaagtgcctaatctgcacacaaaaaatcgtacaatacatgacatcaaggcttaataatagagtgtatgttaaagcgagcatgcaacctatgagtggtatgtaggagttaggtttaaacaaggtaatggctcaagcaccacacatcctaccacaatgtcgtaataaatataaaagcactagcaatctatttagcatgcctaaatgggatactatgaggttgggtgggatgtggcacctttgtataatggcccagttccttagtgtagtcttgatcctccccgttaggttcagactcctctagggattttgtaggaatcatcaaattttcataagcaatttcttgtgcacaaagaaccaaatagattgaaaaagttccaaattcactcaaacacaaaaccatggcacatagcttatgtgacaaaatatttggacactagtttcatattttttgagatcatataagtttattatcaaactccaaggattaaattattttttgaaaaaaaaagaaaaagggaaaacatcataaggtgacacatggcaacctctgaatgactagacttttaccatctctcaggtgggtctggtcaacaatcactgttggtcggtccttaccttgcctagacgggtccttagtaggcctactgggttgagttatgggataaattgtggcctagaaacataccagtccaccaaccttgggaccacttaaaaaattgcatcttgcaccattatactatttagatgtttttaaaaaacaataataacttttacatcgaaatcaaaactagacaaattttatactttcacagagcagcagaaatttatacaatatgattgaatacaagatgtaggacccaatggagagaatttttttgtctcctatatgcttgaatacccaacataatatcttcgcagcatactatctatctaatagaaaaattataatatagttaaatacttaagtagtatctagtggatagaattcaatatctcatacatgcatgaggagtaatatctactagacatgcaacatatttttatctatctaatagaatatatataataaagttaaatattatatgcatcacctactatatataatttgatatcttttagatgtataagggactaagaataatatctctagcacacatgcaatgcattatctatctaaatatattatataatagttaaatattaattatacgtagtctaaacctacatataagcctacccatccccacttaaagatctcagtgtcacacatagaccatacatctcacttcgccaaaaaaatttcgtcaacagttgaagttatacccataatagttaaatattaattatacgtagtctaaacctacatataagcctacccatccccacttaaagatctcagtgtcacacatagaccatacatctcacttcgccaaaaaaatttcgtcaacagttgaagttataccc

SEQ ID NO: 43 - DNA, 63 bp, Zea mays
  source 1..63: mol_type = unassigned DNA
atggcaaaactactcttgggtttgctccttgcccttgctattctagggacaacatcggctgct

SEQ ID NO: 44 - protein, 21 aa, Zea mays
MAKLLLGLLLALAILGTTSAA

SEQ ID NO: 45 - DNA, 720 bp, Aequorea coerulescens
  source 1..720: mol_type = unassigned DNA
atggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtga

SEQ ID NO: 46 - protein, 239 aa, Aequorea coerulescens
MVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYK

SEQ ID NO: 47 - DNA, 23747 bp, Synthetic construct
  misc_feature 1..23747: note = Plasmid
  source 1..23747: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccacgcgtgaagagcggttaccagagctggtcacctttgtccaccaagatggaactgtcttcggccagaatggccatctggattcagcaggcctagaaggccatttatcgctatcaactttgtatagaaaagttgggccgaattcgagctcggtacggccagaatggcccggaccgaagcttcctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaagagccgcaggtcgtttggcgttccatgagcagacactgcagtgagtggatagcaatcaatagcgatgctctttggccttccatgaaacgcacgcactcatgtacgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagcacctaggatccatggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggataaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccagcttcaagtttgtacaaaaaagcaggctccggccagaatggcccggaccgaagcttgcatgcctgcatctagaactaccagcagcatgatcagagtgaggatagttgctacttcaatggcctaggtttgataactactgtcgatccatctgtcgacaattgtttgtttcctctcacaattcacgtttctgccgccacttctttgcacctaacattgcccgtgtcgctgttattagttttgtggttttgcattgtctgtatatagtctgatgaggctatagattgagccttcaaaagtgtttcgttgggcctcttgttgaatgtactccaaagtctcggtgcaccatcagacgtattcctagaatggcacaatctggctatcagaactggtaggtttagtccaaatgtaatgatgtgttctaaagtagtcgggctgagtcaaacagcagcttcttctcttgttcaaaattttgaacctggtaatgttgtcatttcatgatagatcatagatacattcttggtttcactatatcttagaaagtccagccaacagaagtactcaagtacaggtgaacctttcagtttagctggagacatgggcagcagtgtacaaagtctaatccacagctgtgaccctgtgagctgggggttgggcctgcctcggaggggatcgtgtcttttctgtttgtgcaattatcagtgcctaaagaacatgtgttataacagttatacacggtcaattattcaatccatcttttaacgtgtgtttggttgtacgtataagaagggatcaaagagggcggccacattttctataacatttggatgagagtcacacgagaacgagatgaacaccgaagtaattttttatgacagcatgatccaaaaaaatcgagagaacggtgttatccccgtctcatcctattttgatctcaaaccaaacacataattagtcaatgagcactgacttgatgtagaagaacttggtacatataccaggaaatgactctgacttgctgagatgccttgtagtccctcggtcccgtaaattatacagtagtatttaaacttcctcgcacaaaggaaaactatttgtttttgcatgccttgtaaattagctatcaagtttttttttaaaaaaaacgacctttgaaaaccagtaaggaggactcgttcggtattcagttttaaagttcaaggttaaaaatcgaactgacgtgcaaaacaccctgggggttaaaatcaaaacacacaaatttggaaaatgagcaggcatgacttatgttgtgtccgttgaagtgccgtggctccatggcacggtggaactttacattagtgcggtagtgtcactgtggcagtaaagcccacttattttccattagagagagggagattaaaaacgtggctttacacattctgaaactaattgtgagtttgtgactcttggcttgcctgcagtgtccgcctgcaccttctataagtacagtcgcgagcctcagctatacaaaccaactagcgcatagtatctcctgagctccattcatcatcccaagtcttcactggtcttagatcacggatccatggcaccgaagaagaagcgcaaggtgatggacaagaagtacagcatcggcctcgacatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgaccacatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagacaagagaccacgggaccgccacgatggcgagctgggaggccgcaagcgggcaaggtaggtaccgtagttagcttaatcacctagagctcgctatggatcaggatgtggatgtgttcgttcgatcgtttccacgtccaatctcgctgaataaattgtctcgagaaagtttggctttgccgagataaataatagtgataattcttactcctgctacaaactgttttccttgaagcaactatgagcatcttccatagattcgcgacacgatttaaattttaaaaataacatttttaaaaaaatgtgtcacggttacacaactttttttttaacactagttgcgccccgcgtccaagcttactccctgaaattgcattcaaactctcgattcatgtctgcccttctcaccttctgtgtgcctgtgtgggctcgacgtctagtcgtcttcgactcttcgcctacatcagtcacctcctcacacgacgtgtctgcgacacaggccgcggcatagagcacgggcatgtagttttgaacaagtgcatgttgctacctcgactatgatcatgcaaaatattttttttattatttatagaggtttaaaataagacatggaataataagcctggtggccttgaagctggaagtgtagcatggtggaagtgcgggtttgacaggccgacagcgcagagcatatcgaaaatacagctgaaaaaagatccgattatacttgaaaaaaaggataatcaccgaagcaatggaaagctccattcctgtttcccggtggaagaaaaatttctagcctccatccctgcgaacacataggagaactttttatcccttcaccgataaatttatgcccgcgagaaatctgcaaatacaataaatacaaattaattatattaaattaatataaattgaaaaacaagatttaaaattataatagatttctactttagagttcagttagctattttataattagttgtattgaaattttagtaaaattttaacatatcaataagctaaaagaagattatggaacaagtatacggggaattgggggtagaccggttgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgaaggggcttcggaggatgttttagagctagaaatagcaagttaaaataaggctagtccgttatcaacttgaaaaagtggcaccgagtcggtgctttttttttgcggccgcgaattctgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgaaggggcttcggaggatgttttagagctagaaatagcaagttaaaataaggctagtccgttatcaacttgaaaaagtggcaccgagtcggtgctttttttttgaattccaccggtgttaacggcaaacgctaattagtcactaggcccaggaaacactaactaagccctagcttatcctagcgcaacctagttaggtcttggttaccggcctagaaggccgatctcccgggcacccagctttcttgtacaaagtggccgttaacggatcggccagaatggcccggaccgaagctttgtacagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaagcttcctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaagagccgcaggtcgtttggcgttccatgagcagacactgcagtgagtggatagcaatcaatagcgatgctctttggccttccatgaaacgcacgcactcatgtacgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagcacctagtatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcccgggcaactttattatacaaagttgatagataaatcagatatctggttcctaaggacccggcggaccgattaaactgattcggtccgacgcgttacctaataacttcgtatagcatacattatacgaagttattagctaactaactagtaagcttaagccatgggtcgacggatccgttacctgagctgattccgatgacttcgtaggttcctagctcaagccgctcgtgtccaagcgtcacttacgattagctaatgattacggcatctaggaccgactagctaactaactagtacgtacgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 48 - DNA, 27073 bp, Synthetic construct
  misc_feature 1..27073: note = Plasmid
  source 1..27073: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccacgcgtgaagagcggttaccagagctggtcacctttgtccaccaagatggaactgtcttcggccagaatggccatctggattcagcaggcctagaaggccatttatcgctatcaactttgtatagaaaagttgggccgaattcgagctcggtacggccagaatggcccggaccgaagcttcctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaagagccgcaggtcgtttggcgttccatgagcagacactgcagtgagtggatagcaatcaatagcgatgctctttggccttccatgaaacgcacgcactcatgtacgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagcacctaggatccatggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaggtggaggcggctcgggtggcggaggctccggaggcggtggctcgacgtgcgccaaggacatccaaaaagcagccgcggaggcggcactcgcctttcaggacgagacctgcgacaccaccaccaccaaccacggcctcgacatggaggagaccatggtcgaagccatctacacccccgagcagagcgagggcgccttctacatggacgaagagacaatgttcggcatgccaaccctcctcgacaacatggccgaaggcatgctgctgccgcccccgagcgtccagtggaaccacaactacgacggcgaaggcgacggggatgtgtccctgtggagctactagggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggcctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaagagccgcaggtcgtttggcgttccatgagcagacactgcagtgagtggatagcaatcaatagcgatgctctttggccttccatgaaacgcacgcactcatgtacgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagcacctaggatggcgcccagcagctacgaggtggcggcttccatcctcctctgcgccgaggacagcagcagcatcctggacctcgaagcggaggccgaggaggaagcgctactggcgcggagcggggagcccggcggcggcgcggagttccccgtgccgtccgaggaatgcgtcgccggtttcctggagtctgaggcagcgcacatgccgcgggaggactacgccgagaggctgcgcagcggggggatggatctccgcgtccggacggacgccatcgactggatttggaaggttcacacgtgctacggtttcggccctctcactgcctgcttggccgtcaactacctggaccgcttcctctcgctctaccagcttccggaaggcaaggcttggatgacacagctgctgtccgtggcgtgcttgtccctggccgccaagatggaggagacctacgtcccttcgtccctggacctgcaggccggggacgcgcggtacgtgttcgaggcgaagacgatccagaggatggagctcctggtcctcagcacgctcaagtggaggatgcaagcggtcacccccttgtcgtacgtcgactacttcctccaccggctccgaggcggcgccgcgccgagcagacgcgccgtcctccgatccgcggagctcatcctgtgcatcgccagagggacgcactgcttggatttcaggccctcggagatcgccttggccgtcgccgccaccgtggccggcgaagagcgcgccgtggacatcgacagggccttcacccaccgtgtacataaggagagggtgtcgcgatgcctcgaagcgatccagcaggccaccgccaccatggccctgccgcagccgctgaaaagtgaaggcccgagcagcagcggccgcagggcctcgtcttcctcggccaccgtgccgcggagccccaccggggtgctggacgctggctgcctgagctacaggagagccgccgccgccgccgcctcgcacgcgagctccagctggcgcgacgaggacgacgacgactgccccccggctgtctgcagcaagaggaggaagattagcagatgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccagcttcaagtttgtacaaaaaagcaggctccggccagaatggcccggaccgaagcttgcatgcctgcatctagaactaccagcagcatgatcagagtgaggatagttgctacttcaatggcctaggtttgataactactgtcgatccatctgtcgacaattgtttgtttcctctcacaattcacgtttctgccgccacttctttgcacctaacattgcccgtgtcgctgttattagttttgtggttttgcattgtctgtatatagtctgatgaggctatagattgagccttcaaaagtgtttcgttgggcctcttgttgaatgtactccaaagtctcggtgcaccatcagacgtattcctagaatggcacaatctggctatcagaactggtaggtttagtccaaatgtaatgatgtgttctaaagtagtcgggctgagtcaaacagcagcttcttctcttgttcaaaattttgaacctggtaatgttgtcatttcatgatagatcatagatacattcttggtttcactatatcttagaaagtccagccaacagaagtactcaagtacaggtgaacctttcagtttagctggagacatgggcagcagtgtacaaagtctaatccacagctgtgaccctgtgagctgggggttgggcctgcctcggaggggatcgtgtcttttctgtttgtgcaattatcagtgcctaaagaacatgtgttataacagttatacacggtcaattattcaatccatcttttaacgtgtgtttggttgtacgtataagaagggatcaaagagggcggccacattttctataacatttggatgagagtcacacgagaacgagatgaacaccgaagtaattttttatgacagcatgatccaaaaaaatcgagagaacggtgttatccccgtctcatcctattttgatctcaaaccaaacacataattagtcaatgagcactgacttgatgtagaagaacttggtacatataccaggaaatgactctgacttgctgagatgccttgtagtccctcggtcccgtaaattatacagtagtatttaaacttcctcgcacaaaggaaaactatttgtttttgcatgccttgtaaattagctatcaagtttttttttaaaaaaaacgacctttgaaaaccagtaaggaggactcgttcggtattcagttttaaagttcaaggttaaaaatcgaactgacgtgcaaaacaccctgggggttaaaatcaaaacacacaaatttggaaaatgagcaggcatgacttatgttgtgtccgttgaagtgccgtggctccatggcacggtggaactttacattagtgcggtagtgtcactgtggcagtaaagcccacttattttccattagagagagggagattaaaaacgtggctttacacattctgaaactaattgtgagtttgtgactcttggcttgcctgcagtgtccgcctgcaccttctataagtacagtcgcgagcctcagctatacaaaccaactagcgcatagtatctcctgagctccattcatcatcccaagtcttcactggtcttagatcacggatccatggcaccgaagaagaagcgcaaggtgatggacaagaagtacagcatcggcctcgacatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgaccacatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagacaagagaccacgggaccgccacgatggcgagctgggaggccgcaagcgggcaaggtaggtaccgtagttagcttaatcacctagagctcgctatggatcaggatgtggatgtgttcgttcgatcgtttccacgtccaatctcgctgaataaattgtctcgagaaagtttggctttgccgagataaataatagtgataattcttactcctgctacaaactgttttccttgaagcaactatgagcatcttccatagattcgcgacacgatttaaattttaaaaataacatttttaaaaaaatgtgtcacggttacacaactttttttttaacactagttgcgccccgcgtccaagcttactccctgaaattgcattcaaactctcgattcatgtctgcccttctcaccttctgtgtgcctgtgtgggctcgacgtctagtcgtcttcgactcttcgcctacatcagtcacctcctcacacgacgtgtctgcgacacaggccgcggcatagagcacgggcatgtagttttgaacaagtgcatgttgctacctcgactatgatcatgcaaaatattttttttattatttatagaggtttaaaataagacatggaataataagcctggtggccttgaagctggaagtgtagcatggtggaagtgcgggtttgacaggccgacagcgcagagcatatcgaaaatacagctgaaaaaagatccgattatacttgaaaaaaaggataatcaccgaagcaatggaaagctccattcctgtttcccggtggaagaaaaatttctagcctccatccctgcgaacacataggagaactttttatcccttcaccgataaatttatgcccgcgagaaatctgcaaatacaataaatacaaattaattatattaaattaatataaattgaaaaacaagatttaaaattataatagatttctactttagagttcagttagctattttataattagttgtattgaaattttagtaaaattttaacatatcaataagctaaaagaagattatggaacaagtatacggggaattgggggtagaccggttgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgaaggggcttcggaggatgttttagagctagaaatagcaagttaaaataaggctagtccgttatcaacttgaaaaagtggcaccgagtcggtgctttttttttgcggccgcgaattctgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgaaggggcttcggaggatgttttagagctagaaatagcaagttaaaataaggctagtccgttatcaacttgaaaaagtggcaccgagtcggtgctttttttttgaattccaccggtgttaacggcaaacgctaattagtcactaggcccaggaaacactaactaagccctagcttatcctagcgcaacctagttaggtcttggttaccggcctagaaggccgatctcccgggcacccagctttcttgtacaaagtggccgttaacggatcggccagaatggcccggaccgaagctttgtacagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaagcttcctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaagagccgcaggtcgtttggcgttccatgagcagacactgcagtgagtggatagcaatcaatagcgatgctctttggccttccatgaaacgcacgcactcatgtacgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagcacctagtatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcccgggcaactttattatacaaagttgatagataaatcagatatctggttcctaaggacccggcggaccgattaaactgattcggtccgacgcgttacctaataacttcgtatagcatacattatacgaagttattagctaactaactagtaagcttaagccatgggtcgacggatccgttacctgagctgattccgatgacttcgtaggttcctagctcaagccgctcgtgtccaagcgtcacttacgattagctaatgattacggcatctaggaccgactagctaactaactagtacgtacgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 49 - DNA, 1068 bp, Zea mays
  source 1..1068: mol_type = unassigned DNA
atggcgcccagcagctacgaggtggcggcttccatcctcctctgcgccgaggacagcagcagcatcctggacctcgaagcggaggccgaggaggaagcgctactggcgcggagcggggagcccggcggcggcgcggagttccccgtgccgtccgaggaatgcgtcgccggtttcctggagtctgaggcagcgcacatgccgcgggaggactacgccgagaggctgcgcagcggggggatggatctccgcgtccggacggacgccatcgactggatttggaaggttcacacgtgctacggtttcggccctctcactgcctgcttggccgtcaactacctggaccgcttcctctcgctctaccagcttccggaaggcaaggcttggatgacacagctgctgtccgtggcgtgcttgtccctggccgccaagatggaggagacctacgtcccttcgtccctggacctgcaggccggggacgcgcggtacgtgttcgaggcgaagacgatccagaggatggagctcctggtcctcagcacgctcaagtggaggatgcaagcggtcacccccttgtcgtacgtcgactacttcctccaccggctccgaggcggcgccgcgccgagcagacgcgccgtcctccgatccgcggagctcatcctgtgcatcgccagagggacgcactgcttggatttcaggccctcggagatcgccttggccgtcgccgccaccgtggccggcgaagagcgcgccgtggacatcgacagggccttcacccaccgtgtacataaggagagggtgtcgcgatgcctcgaagcgatccagcaggccaccgccaccatggccctgccgcagccgctgaaaagtgaaggcccgagcagcagcggccgcagggcctcgtcttcctcggccaccgtgccgcggagccccaccggggtgctggacgctggctgcctgagctacaggagagccgccgccgccgccgcctcgcacgcgagctccagctggcgcgacgaggacgacgacgactgccccccggctgtctgcagcaagaggaggaagattagcagatga

SEQ ID NO: 50 - protein, 355 aa, Zea mays
MAPSSYEVAASILLCAEDSSSILDLEAEAEEEALLARSGEPGGGAEFPVPSEECVAGFLESEAAHMPREDYAERLRSGGMDLRVRTDAIDWIWKVHTCYGFGPLTACLAVNYLDRFLSLYQLPEGKAWMTQLLSVACLSLAAKMEETYVPSSLDLQAGDARYVFEAKTIQRMELLVLSTLKWRMQAVTPLSYVDYFLHRLRGGAAPSRRAVLRSAELILCIARGTHCLDFRPSEIALAVAATVAGEERAVDIDRAFTHRVHKERVSRCLEAIQQATATMALPQPLKSEGPSSSGRRASSSSATVPRSPTGVLDAGCLSYRRAAAAAASHASSSWRDEDDDDCPPAVCSKRRKISR

SEQ ID NO: 51 - DNA, 26734 bp, Synthetic construct
  source 1..26734: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccacgcgtgaagagcggttaccagagctggtcacctttgtccaccaagatggaactgtcttcggccagaatggccatctggattcagcaggcctagaaggccatttatcgctatcaactttgtatagaaaagttgggccgaattcgagctcggtacggccagaatggcccggaccgaagcttcctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaagagccgcaggtcgtttggcgttccatgagcagacactgcagtgagtggatagcaatcaatagcgatgctctttggccttccatgaaacgcacgcactcatgtacgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagcacctaggatccatggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggataaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggcctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaagagccgcaggtcgtttggcgttccatgagcagacactgcagtgagtggatagcaatcaatagcgatgctctttggccttccatgaaacgcacgcactcatgtacgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagcacctaggatggcgcccagcagctacgaggtggcggcttccatcctcctctgcgccgaggacagcagcagcatcctggacctcgaagcggaggccgaggaggaagcgctactggcgcggagcggggagcccggcggcggcgcggagttccccgtgccgtccgaggaatgcgtcgccggtttcctggagtctgaggcagcgcacatgccgcgggaggactacgccgagaggctgcgcagcggggggatggatctccgcgtccggacggacgccatcgactggatttggaaggttcacacgtgctacggtttcggccctctcactgcctgcttggccgtcaactacctggaccgcttcctctcgctctaccagcttccggaaggcaaggcttggatgacacagctgctgtccgtggcgtgcttgtccctggccgccaagatggaggagacctacgtcccttcgtccctggacctgcaggccggggacgcgcggtacgtgttcgaggcgaagacgatccagaggatggagctcctggtcctcagcacgctcaagtggaggatgcaagcggtcacccccttgtcgtacgtcgactacttcctccaccggctccgaggcggcgccgcgccgagcagacgcgccgtcctccgatccgcggagctcatcctgtgcatcgccagagggacgcactgcttggatttcaggccctcggagatcgccttggccgtcgccgccaccgtggccggcgaagagcgcgccgtggacatcgacagggccttcacccaccgtgtacataaggagagggtgtcgcgatgcctcgaagcgatccagcaggccaccgccaccatggccctgccgcagccgctgaaaagtgaaggcccgagcagcagcggccgcagggcctcgtcttcctcggccaccgtgccgcggagccccaccggggtgctggacgctggctgcctgagctacaggagagccgccgccgccgccgcctcgcacgcgagctccagctggcgcgacgaggacgacgacgactgccccccggctgtctgcagcaagaggaggaagattagcagatgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccagcttcaagtttgtacaaaaaagcaggctccggccagaatggcccggaccgaagcttgcatgcctgcatctagaactaccagcagcatgatcagagtgaggatagttgctacttcaatggcctaggtttgataactactgtcgatccatctgtcgacaattgtttgtttcctctcacaattcacgtttctgccgccacttctttgcacctaacattgcccgtgtcgctgttattagttttgtggttttgcattgtctgtatatagtctgatgaggctatagattgagccttcaaaagtgtttcgttgggcctcttgttgaatgtactccaaagtctcggtgcaccatcagacgtattcctagaatggcacaatctggctatcagaactggtaggtttagtccaaatgtaatgatgtgttctaaagtagtcgggctgagtcaaacagcagcttcttctcttgttcaaaattttgaacctggtaatgttgtcatttcatgatagatcatagatacattcttggtttcactatatcttagaaagtccagccaacagaagtactcaagtacaggtgaacctttcagtttagctggagacatgggcagcagtgtacaaagtctaatccacagctgtgaccctgtgagctgggggttgggcctgcctcggaggggatcgtgtcttttctgtttgtgcaattatcagtgcctaaagaacatgtgttataacagttatacacggtcaattattcaatccatcttttaacgtgtgtttggttgtacgtataagaagggatcaaagagggcggccacattttctataacatttggatgagagtcacacgagaacgagatgaacaccgaagtaattttttatgacagcatgatccaaaaaaatcgagagaacggtgttatccccgtctcatcctattttgatctcaaaccaaacacataattagtcaatgagcactgacttgatgtagaagaacttggtacatataccaggaaatgactctgacttgctgagatgccttgtagtccctcggtcccgtaaattatacagtagtatttaaacttcctcgcacaaaggaaaactatttgtttttgcatgccttgtaaattagctatcaagtttttttttaaaaaaaacgacctttgaaaaccagtaaggaggactcgttcggtattcagttttaaagttcaaggttaaaaatcgaactgacgtgcaaaacaccctgggggttaaaatcaaaacacacaaatttggaaaatgagcaggcatgacttatgttgtgtccgttgaagtgccgtggctccatggcacggtggaactttacattagtgcggtagtgtcactgtggcagtaaagcccacttattttccattagagagagggagattaaaaacgtggctttacacattctgaaactaattgtgagtttgtgactcttggcttgcctgcagtgtccgcctgcaccttctataagtacagtcgcgagcctcagctatacaaaccaactagcgcatagtatctcctgagctccattcatcatcccaagtcttcactggtcttagatcacggatccatggcaccgaagaagaagcgcaaggtgatggacaagaagtacagcatcggcctcgacatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgaccacatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagacaagagaccacgggaccgccacgatggcgagctgggaggccgcaagcgggcaaggtaggtaccgtagttagcttaatcacctagagctcgctatggatcaggatgtggatgtgttcgttcgatcgtttccacgtccaatctcgctgaataaattgtctcgagaaagtttggctttgccgagataaataatagtgataattcttactcctgctacaaactgttttccttgaagcaactatgagcatcttccatagattcgcgacacgatttaaattttaaaaataacatttttaaaaaaatgtgtcacggttacacaactttttttttaacactagttgcgccccgcgtccaagcttactccctgaaattgcattcaaactctcgattcatgtctgcccttctcaccttctgtgtgcctgtgtgggctcgacgtctagtcgtcttcgactcttcgcctacatcagtcacctcctcacacgacgtgtctgcgacacaggccgcggcatagagcacgggcatgtagttttgaacaagtgcatgttgctacctcgactatgatcatgcaaaatattttttttattatttatagaggtttaaaataagacatggaataataagcctggtggccttgaagctggaagtgtagcatggtggaagtgcgggtttgacaggccgacagcgcagagcatatcgaaaatacagctgaaaaaagatccgattatacttgaaaaaaaggataatcaccgaagcaatggaaagctccattcctgtttcccggtggaagaaaaatttctagcctccatccctgcgaacacataggagaactttttatcccttcaccgataaatttatgcccgcgagaaatctgcaaatacaataaatacaaattaattatattaaattaatataaattgaaaaacaagatttaaaattataatagatttctactttagagttcagttagctattttataattagttgtattgaaattttagtaaaattttaacatatcaataagctaaaagaagattatggaacaagtatacggggaattgggggtagaccggttgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgaaggggcttcggaggatgttttagagctagaaatagcaagttaaaataaggctagtccgttatcaacttgaaaaagtggcaccgagtcggtgctttttttttgcggccgcgaattctgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgaaggggcttcggaggatgttttagagctagaaatagcaagttaaaataaggctagtccgttatcaacttgaaaaagtggcaccgagtcggtgctttttttttgaattccaccggtgttaacggcaaacgctaattagtcactaggcccaggaaacactaactaagccctagcttatcctagcgcaacctagttaggtcttggttaccggcctagaaggccgatctcccgggcacccagctttcttgtacaaagtggccgttaacggatcggccagaatggcccggaccgaagctttgtacagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaagcttcctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaagagccgcaggtcgtttggcgttccatgagcagacactgcagtgagtggatagcaatcaatagcgatgctctttggccttccatgaaacgcacgcactcatgtacgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagcacctagtatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcccgggcaactttattatacaaagttgatagataaatcagatatctggttcctaaggacccggcggaccgattaaactgattcggtccgacgcgttacctaataacttcgtatagcatacattatacgaagttattagctaactaactagtaagcttaagccatgggtcgacggatccgttacctgagctgattccgatgacttcgtaggttcctagctcaagccgctcgtgtccaagcgtcacttacgattagctaatgattacggcatctaggaccgactagctaactaactagtacgtacgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 52 - DNA, 27073 bp, Synthetic construct; identical to SEQ ID NO: 48
  source 1..27073: mol_type = other DNA

SEQ ID NO: 53 - DNA, 1858 bp, Arabidopsis thaliana
  source 1..1858: mol_type = unassigned DNA
caggcgtctagagaagctcgctcaaaaacttttgcacgaacacctaaaagttcaatggtccaaaaggacggctctggtcgagagagagagagaagagaggaccgcttcagagaacttcaatcacgaaagatcaaatcacaacccccttatcgtacgcctcttttccttcagtcctttttaacgtacgcatgctctcaatattttccaaagtacaatatagaaagagacggtaataataaaaagacttcatgactttcaaagtacaaaatcctcctcccctgccaaaaaaaaaaaaccctgttgggtctatctttctctctctgtcttctacttcttttgattttaaccttttttcagtagcaagccaaaaaaaaaaaacagacaaagaagttccttttatgataaaggtatgatgatagcaaacaaatgatacccccatgtcttgtgtgtctgcttcatgcaacatgttggtttggatttggttaatctaaaagtttaagataaggttttcggattctcttcctgtcttgtaatagtttcttgtcggagagccatcaacaccaacttcaacaaaaaaaacaagaaaaagaaaaagattctctttctcgttttatttccattagagaagaaaaaaagaatggcgaatccttggtgggtagggaatgttgcgatcggtggagttgagagtccagtgacgtcatcagctccttctttgcaccacagaaacagtaacaacaacaacccaccgactatgactcgttcggatccaagattggaccatgacttcaccaccaacaacagtggaagccctaatacccagactcagagccaagaagaacagaacagcagagacgagcaaccagctgttgaacccggatccggatccgggtctacgggtcgtcgtcctagaggtagacctcctggttccaagaacaaaccaaagagtccagttgttgttaccaaagaaagccctaactctctccagagccatgttcttgagattgctacgggagctgacgtggcggaaagcttaaacgcctttgctcgtagacgcggccggggcgtttcggtgctgagcggtagtggtttggttactaatgttactctgcgtcagcctgctgcatccggtggagttgttagtttacgtggtcagtttgagatcttgtctatgtgtggggcttttcttcctacgtctggctctcctgctgcagccgctggtttaaccatttacttagctggagctcaaggtcaagttgtgggaggtggagttgctggcccgcttattgcctctggacccgttattgtgatagctgctacgttttgcaatgccacttatgagaggttaccgattgaggaagaacaacagcaagagcagccgcttcaactagaagatgggaagaagcagaaagaagagaatgatgataacgagagtgggaataacggaaacgaaggatcgatgcagccgccgatgtataatatgcctcctaattttatcccaaatggtcatcaaatggctcaacacgacgtgtattggggtggtcctccgcctcgtgctcctccttcgtattgattagttagataggcggtggttggtgcgttctttttactggaatgattatattttccattaggatggttaggcttttgtttattaaagctatcaagtttcttttttttttacggataattcggatgacaattagctagtgtttgtttgtttgttttgtggcggcttttctgacttgactattttgatcgcggatagctttgtatgaaagtgaattgattgtagaatcgtcttttgaattttgatgttggaaaaaaccaagcaatggtgtgtggcctttgcaatggaagc

SEQ ID NO: 54 - protein, 310 aa, Arabidopsis thaliana
MANPWWVGNVAIGGVESPVTSSAPSLHHRNSNNNNPPTMTRSDPRLDHDFTTNNSGSPNTQTQSQEEQNSRDEQPAVEPGSGSGSTGRRPRGRPPGSKNKPKSPVVVTKESPNSLQSHVLEIATGADVAESLNAFARRRGRGVSVLSGSGLVTNVTLRQPAASGGVVSLRGQFEILSMCGAFLPTSGSPAAAAGLTIYLAGAQGQVVGGGVAGPLIASGPVIVIAATFCNATYERLPIEEEQQQEQPLQLEDGKKQKEENDDNESGNNGNEGSMQPPMYNMPPNFIPNGHQMAQHDVYWGGPPPRAPPSY

SEQ ID NO: 55 - DNA, 1706 bp, Arabidopsis thaliana
  source 1..1706: mol_type = unassigned DNA
aatatgtcatcaaaattttcccaagaaaacgtacgatgcatattgtatcctatttgtaatacgactttttttctgtcttaaatataattatgtcactctctcaagccttgagttgagggatttgttttaaatttttatttcttataaacactatacgtatacatgtatgtgtatcacatatacatatgcttccacttatttttataagttagcttccattacatcttttctctttcttgcttagtatcattctttgtcgtgttcttttaattaaccttttgcaatttgtcttgtgtttctcacaacacaaaaacttgtaaaagtgttaaaaaatcaagatctgaaaaatcttatcaccgcttctaggtttttcagttttttttcttccttttcctgatctaaattaacttatatttcttagggtttcacttcttgaaacatttaatcagaattaattaacctctctagggctttcatggcgaatccatggtggacaggacaagtgaacctatccggcctcgaaacgacgccgcctggttcctctcagttaaagaaaccagatctccacatctccatgaacatggccatggactcaggtcacaataatcatcaccatcaccaagaagtcgataacaacaacaacgacgacgatagagacaacttgagtggagacgaccacgagccacgtgaaggagccgtagaagcccccacgcgccgtccacgtggacgtcctgctggttccaagaacaaaccaaagccaccgatcttcgtcactcgcgattctccaaatgctctcaagagccatgtcatggagatcgctagtgggactgacgtcatcgaaaccctagctacttttgctaggcggcgtcaacgtggcatctgcatcttgagcggaaatggcacagtggctaacgtcaccctccgtcaaccctcgaccgctgccgttgcggcggctcctggtggtgcggctgttttggctttacaagggaggtttgagattctttctttaaccggttctttcttgccaggaccggctccacctggttccaccggtttaacgatttacttagccggtggtcaaggtcaggttgttggaggaagcgtggtgggcccattgatggcagcaggtccggtgatgctgatcgccgccacgttctctaacgcgacttacgagagattgccattggaggaggaagaggcagcagagagaggcggtggtggaggcagcggaggagtggttccggggcagctcggaggcggaggttcgccactaagcagcggtgctggtggaggcgacggtaaccaaggacttccggtgtataatatgccgggaaatcttgtttctaatggtggcagtggtggaggaggacagatgagcggccaagaagcttatggttgggctcaagctaggtcaggattttaacgtgcgttaaaatggtttttaatttacagaagttaacaataagattataatgatgtttattatgatgatgaaaaccagtcagttgctacttgttactagtgagctatatagtttgtggacattatattatgttctctcttgactatgattattatttgctaaatttcacttagcttacttgtgtttttatgatgttttattaaaagtttttctctttgttggattctccaattttttatagtgaggttaagtgtgcatacatatatacatgtgtacatgattg

SEQ ID NO: 56 - protein, 315 aa, Arabidopsis thaliana
MANPWWTGQVNLSGLETTPPGSSQLKKPDLHISMNMAMDSGHNNHHHHQEVDNNNNDDDRDNLSGDDHEPREGAVEAPTRRPRGRPAGSKNKPKPPIFVTRDSPNALKSHVMEIASGTDVIETLATFARRRQRGICILSGNGTVANVTLRQPSTAAVAAAPGGAAVLALQGRFEILSLTGSFLPGPAPPGSTGLTIYLAGGQGQVVGGSVVGPLMAAGPVMLIAATFSNATYERLPLEEEEAAERGGGGGSGGVVPGQLGGGGSPLSSGAGGGDGNQGLPVYNMPGNLVSNGGSGGGGQMSGQEAYGWAQARSGF

SEQ ID NO: 57 - DNA, 1815 bp, Arabidopsis thaliana
  source 1..1815: mol_type = unassigned DNA
ccagtatattttatatattaataaaaagagagtacacacatagtattgcgttcttggattctatataaaccctatataaataatactaacttgtgaaaaattaattattgcccactttctctctctctctctctctgattgattcttgaaaagaaaatcaaagtctcaatagctttgttttgattctagttccattccacaattgcatctaattctcctaccgtagccttttcttctttatttgtcatcaacaactcctaaaattcatatgaaattagagaaatcgaaacgaaaagataatatttttcaagaaagagctacatatacaccttttgaccttgtaaagttggaaccctagaggcctttcaagcaaatcatcagggtaacaatttcttgatctttctttttagcgaatttccagtttttggtcaatcatggcaaacccttggtggacgaaccagagtggtttagcgggcatggtggaccattcggtctcctcaggccatcaccaaaaccatcaccaccaaagtcttcttaccaaaggagatcttggaatagccatgaatcagagccaagacaacgaccaagacgaagaagatgatcctagagaaggagccgttgaggtggtcaaccgtagaccaagaggtagaccaccaggatccaaaaacaaacccaaagctccaatctttgtgacaagagacagccccaacgcactccgtagccatgtcttggagatctccgacggcagtgacgtcgccgacacaatcgctcacttctcaagacgcaggcaacgcggcgtttgcgttctcagcgggacaggctcagtcgctaacgtcaccctccgccaagccgccgcaccaggaggtgtggtctctctccaaggcaggtttgaaatcttatctttaaccggtgctttcctccctggaccttccccacccgggtcaaccggtttaacggtttacttagccggggtccagggtcaggtcgttggaggtagcgttgtaggcccactcttagccatagggtcggtcatggtgattgctgctactttctctaacgctacttatgagagattgcccatggaagaagaggaagacggtggcggctcaagacagattcacggaggcggtgactcaccgcccagaatcggtagtaacctgcctgatctatcagggatggccgggccaggctacaatatgccgccgcatctgattccaaatggggctggtcagctagggcacgaaccatatacatgggtccacgcaagaccaccttactgactcagtgagccatttctatatataatggtctatataaataaatatatagatgaatataagcaagcaatttgaggtagtctattacaaagcttttgctctggttggaaaaataaataagtatcaaagctttgtttgttcttaatggaaatatagagcttgggaaggtagaaagagacgacattaagggactttcagattttcagtattactactccacgaaattattgaaataaataaacattgtactgtaaattagaatgtttgtctttttgtttttgtgtagtagtatttaactacaatatttgtatgtgtttgcctttgtgatctttagatactttacattttggttggctcaaaaaccatgggtttattatatatatttaatgctggttcaacgatgaaggaacgttgagagcatatattgcctatttgtgcgtttcgatattatatttctaattcatgatcatttttatttaaggtttttaccataggtccatagtggtggttctttaatcttcgcgtcatgatgctattg

SEQ ID NO: 58 - protein, 281 aa, Arabidopsis thaliana
MANPWWTNQSGLAGMVDHSVSSGHHQNHHHQSLLTKGDLGIAMNQSQDNDQDEEDDPREGAVEVVNRRPRGRPPGSKNKPKAPIFVTRDSPNALRSHVLEISDGSDVADTIAHFSRRRQRGVCVLSGTGSVANVTLRQAAAPGGVVSLQGRFEILSLTGAFLPGPSPPGSTGLTVYLAGVQGQVVGGSVVGPLLAIGSVMVIAATFSNATYERLPMEEEEDGGGSRQIHGGGDSPPRIGSNLPDLSGMAGPGYNMPPHLIPNGAGQLGHEPYTWVHARPPY

SEQ ID NO: 59 - DNA, 822 bp, Zea mays
  source 1..822: mol_type = unassigned DNA
atggggagcctcgacggcggccactcgctgcagcagggccgccaccacggctacgcccactcccactcccacgtcggcgcgggtcccgacagcagcaacaacaacaacgacgaggacgacgcgtcgcccccgccgcctgccggggcgggaggaggtcctcgccggccgcgcgggaggcccccggggtccaagaacaagccgaagccgcccgtggtggtgacgcgggagagccccaacgcgatgcgctcccacgtgctggagatcgccagcggcgccgacatcgtcgacgccatcgcgggcttctcccgccgccgccagcgcggcgtctccgtcctcagcggcacgggcgcggtcaccaacgtcacgctgcgggagcccgcgggggcaggcggggcagcggccgtcgcgctgcgcggccgcttcgagatactgtccatgtccggggctttcctcccggcgccggcgccgccgggggccacggggctcaccgtgtacctggcgggggggcaggggcaggtggtgggcgggagcgtcatgggggagctcatcgcttcgggccccgtcatggtgatcgccgccacgttcggcaacgccacctacgagaggctgccgctggaccaggcagacgccgaggagggcgccgtgctgtccggctcgtccgagggcgccaccgcgcagcagctggagcagcagcagagcagcgggggtcccgtcgtgccgccgtccatgtacgccgtcccgcagacgccgccgcacccgcacgacatgttcgggcagtgggggcacgcggctgtgacgcgcccaccgccgacgtcgttctag

SEQ ID NO: 60 - protein, 273 aa, Zea mays
MGSLDGGHSLQQGRHHGYAHSHSHVGAGPDSSNNNNDEDDASPPPPAGAGGGPRRPRGRPPGSKNKPKPPVVVTRESPNAMRSHVLEIASGADIVDAIAGFSRRRQRGVSVLSGTGAVTNVTLREPAGAGGAAAVALRGRFEILSMSGAFLPAPAPPGATGLTVYLAGGQGQVVGGSVMGELIASGPVMVIAATFGNATYERLPLDQADAEEGAVLSGSSEGATAQQLEQQQSSGGPVVPPSMYAVPQTPPHPHDMFGQWGHAAVTRPPPTSF

SEQ ID NO: 61 - DNA, 1026 bp, Zea mays
  source 1..1026: mol_type = unassigned DNA
atggatcccgtggcggcggcgcacggcggcggccaccacttcgcgccgccagtggcgccgttccaccacgcgttcggcggcggccaccacctgttcccgggacagcagcacccggcgttccagcatttccaggagcaccagctggagctcatgggcggggtccccgccggcgcgaagcaggagctcggcgacgacagcaacaacaccatcaacagcgcggggagcaacgggagcggcgacggcgaccagcagcagcagcagcagcaggccgggggcgacgtcgacaaccaccaccagcaccaccaccaccagcaggccgcggtgatgcgccgcccgcggggtcgccccgcggggtccaagaacaagccgaagccgcccgtgatcatcacgcgggacagcgccagcgcgctgcgcgcgcacgtgctggaggtggccgccggctgcgacgtggtggacagcgtggccggcttcgcgcggcgccgccaggtgggcgtgtgcgtgctcagcggcgccggcagcgtggccaacgtgtgcgtccgccagcccggcgccggcgccggggccgtcacgctgccgggccgcttcgagatcctctccctctgcggctccttcctcccgccgccggcgccgcccgccgccacgggcctcaccgtctacctctccggcgggcagggccaggtggtgggcgggtccgtggccgggccgctcctcgcgtccggccccgtcgtcatcgtcgccgcctgcttcggcaacgccgcctacgagcgcctcccgctcgacgacgacgacctgccgcagcagcagcagcacctgccccctcctcctcctattccgcagggcatgcccggagctgccggcgccggcgggcagtcctcgtccccgccgcccccgccgctgccattgcccggacaacacccgctcgccgaccagctcccccacggcctcatcaacggcctccctctccccgctgacgcctacggctgggccaacccaggaggcggcgccggccgcgtcgccccgtactga

SEQ ID NO: 62 - protein, 341 aa, Zea mays
MDPVAAAHGGGHHFAPPVAPFHHAFGGGHHLFPGQQHPAFQHFQEHQLELMGGVPAGAKQELGDDSNNTINSAGSNGSGDGDQQQQQQQAGGDVDNHHQHHHHQQAAVMRRPRGRPAGSKNKPKPPVIITRDSASALRAHVLEVAAGCDVVDSVAGFARRRQVGVCVLSGAGSVANVCVRQPGAGAGAVTLPGRFEILSLCGSFLPPPAPPAATGLTVYLSGGQGQVVGGSVAGPLLASGPVVIVAACFGNAAYERLPLDDDDLPQQQQHLPPPPPIPQGMPGAAGAGGQSSSPPPPPLPLPGQHPLADQLPHGLINGLPLPADAYGWANPGGGAGRVAPY

SEQ ID NO: 63 - DNA, 930 bp, Zea mays
  source 1..930: mol_type = unassigned DNA
atggccggcctcgacctcggcaccgctgcgacgcgctacgttcatcagctccaccacctccaccccgacctccagctgcagcacaactacgccaagcagccggagccatccgaggacgaccctaatggcggcggcggcggcaacagcaacaacggcggaccgtatggtgagcacgacggcgggtcgtcctcatccggcccagctggagatgcccccggtggcagcggcggtaacggggagatggtagtccgccggccccgcggacgcccaccgggctcgaagaataagccgaagccgccggtgatcatcacgcgggagagcgccaatacgctgcgcgcccacatcctggaggtggcgagcggctgcgacgtgttcgagagtgtctccacgtatgcgcgccggcggcagcgtggcgtctgcgtgctgagcggcagcggcgtcgtcaccaacgtgacgctacggcagccgtcggcgcccgcgggcgccgtggtgacgctgcacgggaggttcgagatcctgtcgctctcgggatctttcctcccgccgccggctccccccggcgccaccagcctcaccatcttcctcgcggggggccaggggcaggtggtcggtgggaacgtcgtcggtgcgctctacgccgcaggccccgtcatcgtcatcgccgcgtccttcgccaacgtcgcctacgagcgcctccccctggaggaggaagaggctcaggcagcgccaccgggcctccagatgcagccacccggcggtggtgtcgatggcgctggaggcatgggcggtgggccgttccctcccgatccgtcggcggctgccgggctgccgttcttcaacctgccgctcaacaacatgtccggtggcgggtcgccgctcccgcccggtgctgacggccatggttgggccggtgcacggccgccgttctga

SEQ ID NO: 64 - protein, 309 aa, Zea mays
MAGLDLGTAATRYVHQLHHLHPDLQLQHNYAKQPEPSEDDPNGGGGGNSNNGGPYGEHDGGSSSSGPAGDAPGGSGGNGEMVVRRPRGRPPGSKNKPKPPVIITRESANTLRAHILEVASGCDVFESVSTYARRRQRGVCVLSGSGVVTNVTLRQPSAPAGAVVTLHGRFEILSLSGSFLPPPAPPGATSLTIFLAGGQGQVVGGNVVGALYAAGPVIVIAASFANVAYERLPLEEEEAQAAPPGLQMQPPGGGVDGAGGMGGGPFPPDPSAAAGLPFFNLPLNNMSGGGSPLPPGADGHGWAGARPPF

SEQ ID NO: 65 - DNA, 1687 bp, Zea mays
  source 1..1687: mol_type = unassigned DNA
atctctctcccatcattcatctatctgatctcatctcctatcttccctctctctcctccccgtcgtctgccttctctgcccggcaagaccgcctgcccatgtccgtggcgccgtgcgcgcttctcctgccctgcaatccgcttcctcagcgcagctccgatccgccaacgccgcccaaggagccgggatcggccgaccgatcggagacgagaggcaggcagcggaacagagcagcggcccgcaaggcgattgctctattctgccctgttccagcgttccatgcatggatgccaaggaactcatctcacagtccgacctgcagtcgttctaccagcagcaacaacagcagcaccgtgccttcggcggccaccatagcccgtcctcgctggccgggatgcactccgtcatccgacccatgcccaacatgaatatgagcccaactgctatccttaactccatcagcgggggttccctcgccggcatgcagttccagatggacccgcccccaccattgctgcacaacatcaacgccaccggcgctgtcacaccggcccctgcggagcctctcaagcgcaaaagaggaaggccgcgcaaatacggacccgatggcacgatgaggcaacagcaacagcaacaggctgcgtcatcgcagcagcagctcgtcgctacacaaccgaggatatgttctctatcgtcgggtcccgacatgctcggcagctccggtatggaggaccccgcgcagaagaagcggcgcggccggccgccgggcaccggcaagaagcaccagccctcgacctcacaaggtccaggcaatgcgttcgccggttcggctggaacgagcttcactccgcacatcatcacggcgtctccttcagaggacgtggcggcgaagatcgttgcgttcgccagccagtcgtcaaaggcggtgtgcgtcctctcggcgatgggttccgtctccagggccgtcctccgccacccggccgacggctctcccatggcgagggttcacgcgtcgccgcagccgtacaagaaccctgctgtctacgagggtttctacgagatcctgagcctaaccggctcgtacaacctggcacagggcggcggcctcagcgtcacgctctgcagccctgagaggaacgtgatcggaggcgttctgggcggcccactggtagctgcaggcaccgtgcaggtggttctggggagttttcaccagggagggtcgagatccaagtccaagaaagccgggaagcagcagcaggcggcggccttcagccccgactcgctcaccggcggccaggaggcgtcgcccagctccgggcacaaccaaaatctaacgccgccgccctccgtgacgggagggtggcccacctccggcatatttgacaccagaagttccagcattgatatcaactcgtctagaggctagctcattcgtcgatataattccagctcgacagaaattttctgttttcgcacaagaatacccaaaaaccatacttcgaaatagcttattcgcttctttcttttgagtaattaaattcgtatagaattcataggcatcgctcagtctcgtgacttgttattattcttccaacaatcccccttcgtcctctactcctctccgcgtggcataccgttgtgcaaattatacttcagaaatgcaaatatgcaactggtgtatgtaaat

SEQ ID NO: 66 - protein, 380 aa, Zea mays
MDAKELISQSDLQSFYQQQQQQHRAFGGHHSPSSLAGMHSVIRPMPNMNMSPTAILNSISGGSLAGMQFQMDPPPPLLHNINATGAVTPAPAEPLKRKRGRPRKYGPDGTMRQQQQQQAASSQQQLVATQPRICSLSSGPDMLGSSGMEDPAQKKRRGRPPGTGKKHQPSTSQGPGNAFAGSAGTSFTPHIITASPSEDVAAKIVAFASQSSKAVCVLSAMGSVSRAVLRHPADGSPMARVHASPQPYKNPAVYEGFYEILSLTGSYNLAQGGGLSVTLCSPERNVIGGVLGGPLVAAGTVQVVLGSFHQGGSRSKSKKAGKQQQAAAFSPDSLTGGQEASPSSGHNQNLTPPPSVTGGWPTSGIFDTRSSSIDINSSRG

SEQ ID NO: 67 - DNA, 900 bp, Zea mays
  source 1..900: mol_type = unassigned DNA
atggcagacctggatttgggcgccagctcctacctgcaccagcagcaccaccaccagcagctactgcccctccacctccaccacgaagacggcggcggcgccgcggggggctccgacgacggccaggactcgctgtcgccgggatcgggcggcggtgggggtccgcccagcaccgccggaggcgcgggcatcggcggcggggacgtcgtcgcgcgccgcccgcgcggccgcccgccggggtccaagaacaagcccaagccgcccgtgatcatcaccagggagagcgccaacgcgctccgcgcgcacatcctcgaggtcgccgccggctgcgacgtcttcgaggcgctcaccgcctacgcgcgccgcaggcagcgcggcgtctgcgttctctcggcggccgggaccgtggccaacgtcacgctgcgccagccgcagtcggcccagacgggcccggcggcctcggcgccggcggtcgccacgctccagggccggttcgagatcctctccctcgccggctccttcctgccgccgcccgcgcccccgggggccaccagcctcgccgccttcctggccgcggggcagggccaggttgtcggtggcagcgtggccggcgcgctcatcgctgcggggccggtcgtcgtcgtcgccgcgtcgttcagcaacgtcgcctacgagaggctgccgctcgaggagggcgacgaggtggctcctcctccgccgcctgcggggaccgaccagcagcctgccggtatgccgtttgaccccgcggccgcggctgccgctggcgggcttcccttcttcaacctgcctttggggatgccgccaatgccgatggacgaccacgccggctggcctgctggcgcccctggaggtggtgttgggaggtcgcccttctcgtga

SEQ ID NO: 68 - protein, 299 aa, Zea mays
MADLDLGASSYLHQQHHHQQLLPLHLHHEDGGGAAGGSDDGQDSLSPGSGGGGGPPSTAGGAGIGGGDVVARRPRGRPPGSKNKPKPPVIITRESANALRAHILEVAAGCDVFEALTAYARRRQRGVCVLSAAGTVANVTLRQPQSAQTGPAASAPAVATLQGRFEILSLAGSFLPPPAPPGATSLAAFLAAGQGQVVGGSVAGALIAAGPVVVVAASFSNVAYERLPLEEGDEVAPPPPPAGTDQQPAGMPFDPAAAAAAGGLPFFNLPLGMPPMPMDDHAGWPAGAPGGGVGRSPFS

SEQ ID NO: 69 - DNA, 213 bp, Arabidopsis thaliana
  source 1..213: mol_type = unassigned DNA
ggcttcgacatggaggaaacgctcgttgaagccatctacaccgccgagcagagcgagaacgccttctacatgcacgacgaggccatgttcgagatgccctcactgctcgccaacatggccgaaggcatgctcctgcccctgcccagcgtgcagtggaaccataaccacgaagttgacggcgatgacgacgatgtgtctctgtggtcgtactag

SEQ ID NO: 70 - protein, 70 aa, Arabidopsis thaliana
GFDMEETLVEAIYTAEQSENAFYMHDEAMFEMPSLLANMAEGMLLPLPSVQWNHNHEVDGDDDDVSLWSY

SEQ ID NO: 71 - DNA, 243 bp, Herpes simplex virus
  source 1..243: mol_type = unassigned DNA
tcctctgcgccgcccacggacgtgtcattaggagatgaattacatcttgatggtgaggatgtcgcgatggcccacgcggacgcgcttgatgatttcgacctcgacatgcttggggacggcgactcccccggtcctggcttcacgcctcatgattcagctccgtacggagcgctggacatggctgacttcgaatttgagcagatgttcacagatgcactcggcatagatgagtacggtgggtag

SEQ ID NO: 72 - protein, 80 aa, Herpes simplex virus
SSAPPTDVSLGDELHLDGEDVAMAHADALDDFDLDMLGDGDSPGPGFTPHDSAPYGALDMADFEFEQMFTDALGIDEYGG

SEQ ID NO: 73 - DNA, 246 bp, Herpes simplex virus
  source 1..246: mol_type = unassigned DNA
atttcgaccaccgcgcccccgaccgatgtttcgcttggggacgaacttcatcttgacggtgaggatgttgcaatggcgcacgctgatgcgttggatgatttcgacctcgacatgcttggggatggtgactccccagggcccggttttacgcctcacgactcggcgccctatggcgcacttgatgtggacttcgaatttgagcaaatgttcacggatgcgttgggtattgacgattttggcgggtag

SEQ ID NO: 74 - protein, 81 aa, Herpes simplex virus
ISTTAPPTDVSLGDELHLDGEDVAMAHADALDDFDLDMLGDGDSPGPGFTPHDSAPYGALDVDFEFEQMFTDALGIDDFGG

(19) United States
(12) Patent Application Publication
(10) Pub. No.: US 2025/0101453 A1
(43) Pub. Date: Mar. 27, 2025

(54) METHODS OF PARTHENOGENIC HAPLOID INDUCTION AND HAPLOID CHROMOSOME DOUBLING

(71) Applicant: PIONEER HI-BRED INTERNATIONAL, INC., JOHNSTON, IA (US)

(72) Inventors: WILLIAM JAMES GORDON-KAMM, URBANDALE, IA (US); JON AARON TUCKER REINDERS, CLIVE, IA (US); NAGESH SARDESAI, JOHNSTON, IA (US); HUAXUN YE, JOHNSTON, IA (US)

(73) Assignee: PIONEER HI-BRED INTERNATIONAL, INC., JOHNSTON, IA (US)

(21) Appl. No.: 18/850,498

(22) PCT Filed: Mar. 24, 2023
(86) PCT No.: PCT/US23/64929

§ 371 Date: Sep. 24, 2024

Related U.S. Application Data
(60) Provisional application No. 63/269,940, filed on Mar. 25, 2022.

(51) Int. Cl. C12N 15/82; C07K 14/415

(52) U.S. Cl. CPC C12N 15/8287; C07K 14/415; C07K 2319/00

(57) ABSTRACT

The present disclosure provides methods of generating doubled haploid plants using a polynucleotide sequence encoding the truncated ZM-ODP2 polypeptide for parthenogenic haploid induction and haploid chromosome doubling.

### REFERENCE TO SEQUENCE LISTING SUBMITTED ELECTRONICALLY

The official copy of the sequence listing is submitted electronically via Patent Center as an XML formatted sequence listing with a file named “8963-WO-PCT_Sequence_Listing_ST26” created on Mar. 20, 2023 and having a size of 347 KB and is filed concurrently with the specification. The sequence listing contained in this ASCII formatted document is part of the specification and is incorporated by reference herein in its entirety.

### BACKGROUND OF THE DISCLOSURE

Plant breeding programs identify new cultivars by screening numerous plants to identify individuals with desirable characteristics. Large numbers of progeny from crosses are typically grown and evaluated, ideally across multiple years and environments, to select the plants with the most desirable characteristics.

Typical breeding methods cross two parental plants and the filial 1 hybrid (F₁ hybrid), is the first filial (F₁) generation. Hybrid vigor in a commercial F₁ hybrid is observed when two parental strains, (typically inbreds), from different heterotic groups are intercrossed. Hybrid vigor, the improved or increased function of any biological quality resulting from combining the genetic contributions of its parents, is important to commercial maize seed production. Commercial hybrid performance improvements require continued development of new inbred parental lines.

Maize inbred line development methods may use maternal (gynogenic) doubled haploid production, in which maternal haploid embryos are selected following the fertilization of the ear of a plant resultant from a first-generation cross that has been fertilized with pollen from a so-called “haploid inducer” line. Pollination of a female flower with pollen of a haploid inducer strain results in elevated levels of ovules that contain only the haploid maternal genome, as opposed to inheriting a copy of both the maternal and paternal genome, thus, creating maternal haploid embryos. Ovules within the female flower are the products of meiosis and each maternal ovule is a unique meiotically recombined haploid genome, thereby allowing immature maternal haploid embryos to be isolated and treated using in vitro tissue culture methods that include chromosome doubling treatments to rapidly enable generating maternal doubled haploid recombinant populations. Many of the maize maternal haploid embryos generated by fertilizing a target plant with pollen from a maize haploid inducer line fail to regenerate into a fertile, doubled haploid plant and few, if any, in vitro tissue culture and plantlet regeneration methods propagate multiple, fertile plants from one haploid embryo. Thus, there is a need for improving methods of producing doubled haploid plants from maternal gamete doubled haploids in maize.

Plant breeders would thus also benefit from methods of developing a population of recombinant inbred lines that do not require extensive pollination control methods or the prolonged time required for propagating self-fertilized lines into isogenic states.

### SUMMARY

In a first aspect, the disclosure provides a method of producing a doubled haploid plant, the method comprising: providing a diploid embryo of a plant with a polynucleotide sequence encoding at least a truncated ZM-ODP2 polypeptide; regenerating a T₀ plant from the diploid embryo, wherein the T₀ plant expresses the polynucleotide sequence encoding the truncated ZM-ODP2 polypeptide; obtaining a donor ear from the T₀ plant; pollinating the donor ear with pollen from a pollen donor plant; selecting a haploid embryo expressing the polynucleotide sequence encoding the truncated ZM-ODP2 polypeptide and lacking the genome of the haploid inducer plant, wherein the truncated ZM-ODP2 polypeptide promotes chromosome doubling of the haploid embryo to produce a doubled haploid embryo; and regenerating a doubled haploid plant from the doubled haploid embryo or a mature seed thereof.

In some aspects of the method, chromosome doubling is achieved without a chemical chromosome doubling agent.

In some aspects of the method, a second genetic chromosome doubling agent is provided to the diploid embryo of the plant along with the polynucleotide sequence encoding the truncated ZM-ODP2 polypeptide. In some aspects of the method, the second genetic chromosome doubling agent comprises a polynucleotide sequence encoding a cyclin gene family member.

In some aspects of the method, the pollen donor plant is a non-haploid inducer plant.

In some aspects of the method, the pollen donor plant is a haploid inducer plant selected and/or derived from lines Stock 6, RWS, KEMS, KMS, or ZMS.

In some aspects of the method, the pollen donor plant comprises a paternal marker gene that is expressed in embryo tissue. In some aspects, the marker gene is a morphological marker, for example, a morphological marker expressing anthocyanin pigments. In some aspects, the marker gene is a reporter gene expressing a fluorescent protein, for example, GFP, YFP, CFP, or RFP.

In some aspects of the method, the polynucleotide sequence encoding the truncated ZM-ODP2 polypeptide is selected from: a polynucleotide sequence that has at least 85% sequence identify to SEQ ID NO: 1; a polynucleotide sequence that has at least 95% sequence identify to SEQ ID NO: 1; and a polynucleotide sequence that has SEQ ID NO: 1; and the truncated ZM-ODP2 polypeptide is selected from: a polypeptide sequence having at least 85% sequence identity to SEQ ID NO: 2; a polypeptide sequence having at least 95% sequence identity to SEQ ID NO: 2; and a polypeptide sequence having SEQ ID NO:2.

In some aspects of the method, the truncated ZM-ODP2 polypeptide is part of a fusion protein that further comprises CBF1a, GNAT1, GNAT2, HAT1, HAT2, JMJ, or SV40:VP64.

In some aspects of the method, providing the diploid embryo of the plant with the polynucleotide sequence encoding at least the truncated ZM-ODP2 polypeptide comprises providing the diploid embryo of the plant with a polynucleotide sequence encoding a fusion protein, the fusion protein comprising the truncated ZM-ODP2 polypeptide and CBF1a, GNAT1, GNAT2, HAT1, HAT2, JMJ, or SV40:VP64. In some aspects, the fusion protein exhibits cell non-autonomous activity.

In another aspect, the disclosure provides a method of producing a doubled haploid plant, the method comprising: stimulating parthenogenic haploid induction and chromosome doubling by providing a haploid plant cell with a polynucleotide sequence encoding at least a truncated ZM-ODP2 polypeptide; regenerating a T₀ plant expressing the polynucleotide sequence encoding the truncated ZM-ODP2 polypeptide, wherein a haploid set of chromosomes is diploidized; pollinating the T₀ plant; obtaining a doubled haploid embryo from the T₀ plant; and regenerating a doubled haploid plant from the doubled haploid embryo or a mature seed thereof.

In some aspects of the method, diploidization is achieved without a chemical chromosome doubling agent.

In some aspects of the method, a second genetic chromosome doubling agent is provided to the haploid plant cell along with the polynucleotide sequence encoding the truncated ZM-ODP2 polypeptide. In some aspects, the second genetic chromosome doubling agent comprises a polynucleotide sequence encoding a cyclin gene family member.

In some aspects of the method, pollinating the T0 plant comprises self-pollination.

In some aspects of the method, pollinating the T0 plant comprises pollinating the T0 plant with pollen from a sister plant.

In some aspects of the method, the polynucleotide sequence encoding the truncated ZM-ODP2 polypeptide is selected from: a polynucleotide sequence that has at least 85% sequence identify to SEQ ID NO: 1; a polynucleotide sequence that has at least 95% sequence identify to SEQ ID NO: 1; and a polynucleotide sequence that has SEQ ID NO: 1; and the truncated ZM-ODP2 polypeptide is selected from: a polypeptide sequence having at least 85% sequence identity to SEQ ID NO: 2; a polypeptide sequence having at least 95% sequence identity to SEQ ID NO: 2; and a polypeptide sequence having SEQ ID NO:2.

In some aspects of the method, the truncated ZM-ODP2 polypeptide is part of a fusion protein that further comprises CBF1a, GNAT1, GNAT2, HAT1, HAT2, JMJ, or SV40:VP64.

In some aspects of the method, providing the diploid embryo of the plant with the polynucleotide sequence encoding at least the truncated ZM-ODP2 polypeptide comprises providing the diploid embryo of the plant with a polynucleotide sequence encoding a fusion protein, the fusion protein comprising the truncated ZM-ODP2 polypeptide and CBF1a, GNAT1, GNAT2, HAT1, HAT2, JMJ, or SV40:VP64. In some aspects, the fusion protein exhibits cell non-autonomous activity.

In another aspect, the disclosure provides, a method of producing a genome-edited doubled haploid plant, the method comprising: providing a diploid embryo of a plant with (i) a polynucleotide sequence encoding at least a truncated ZM-ODP2 polypeptide; and (ii) at least one polynucleotide sequence encoding at least one genome-editing component; regenerating a T₀ plant from the diploid embryo, wherein the T₀ plant expresses the polynucleotide sequence encoding at least the truncated ZM-ODP2 polypeptide and the at least one polynucleotide sequence encoding the at least one genome-editing component; obtaining a donor ear from the T₀ plant; pollinating the donor ear with pollen from a pollen donor; selecting a haploid embryo that expresses the polynucleotide sequence encoding the truncated ZM-ODP2 polypeptide and the at least one polynucleotide sequence encoding the at least one genome-editing component, and lacks the genome of the haploid inducer plant, wherein the truncated ZM-ODP2 polypeptide promotes chromosome doubling of the haploid embryo to produce a doubled haploid embryo; and regenerating a doubled haploid plant from the doubled haploid embryo or a mature seed thereof.

In some aspects of the method, chromosome doubling is achieved without a chemical chromosome doubling agent.

In some aspects of the method, a second genetic chromosome doubling agent is provided to the diploid embryo of the plant along with the polynucleotide sequence encoding the truncated ZM-ODP2 polypeptide. In some aspects, the second genetic chromosome doubling agent comprises a polynucleotide sequence encoding a cyclin gene family member.

In some aspects of the method, the pollen donor plant is a non-haploid inducer plant.

In some aspects of the method, the pollen donor is a haploid inducer plant selected and/or derived from lines Stock 6, RWS, KEMS, KMS, or ZMS.

In some aspects of the method, the pollen donor plant comprises a paternal marker gene that is expressed in embryo tissue. In some aspects, the marker gene is a morphological marker, for example, a morphological marker expressing anthocyanin pigments. In some aspects, the marker gene is a reporter gene expressing a fluorescent protein, for example, GFP, YFP, CFP, or RFP.

In some aspects of the method, the polynucleotide sequence encoding the truncated ZM-ODP2 polypeptide is selected from: a polynucleotide sequence that has at least 85% sequence identify to SEQ ID NO: 1; a polynucleotide sequence that has at least 95% sequence identify to SEQ ID NO: 1; and a polynucleotide sequence that has SEQ ID NO: 1; and the truncated ZM-ODP2 polypeptide is selected from: a polypeptide sequence having at least 85% sequence identity to SEQ ID NO: 2; a polypeptide sequence having at least 95% sequence identity to SEQ ID NO: 2; and a polypeptide sequence having SEQ ID NO:2.

In some aspects of the method, the truncated ZM-ODP2 polypeptide is part of a fusion protein that further comprises CBF1a, GNAT1, GNAT2, HAT1, HAT2, JMJ, or SV40:VP64.

In some aspects of the method, providing the diploid embryo of the plant with the polynucleotide sequence encoding at least the truncated ZM-ODP2 polypeptide comprises providing the diploid embryo of the plant with a polynucleotide sequence encoding a fusion protein, the fusion protein comprising the truncated ZM-ODP2 polypeptide and CBF1a, GNAT1, GNAT2, HAT1, HAT2, JMJ, or SV40:VP64. In some aspects, the fusion protein exhibits cell non-autonomous activity.

In some aspects of the method, the at least one genome-editing component is a site-directed nuclease selected from meganucleases (MNs), zinc-finger nucleases (ZFN), transcription-activator like effector nucleases (TALENs), Cas9 nuclease, Cas alpha nuclease, Cpf1 nuclease, dCas9-FokI, dCpf1-FokI, chimeric Cas9-cytidine deaminase, chimeric Cas9 adenine deaminase, chimeric FEN1-Fok1, Mega-TALs, a nickase Cas9 (nCas9), chimeric dCas9 non-FokI nuclease, and dCpf1-non-FokI nuclease.

### Cas-Alpha Endonuclease

In some aspects of the methods disclosed herein, a genome editing system comprises a Cas-alpha (e.g., Cas12f) endonuclease and one or more guide polynucleotides that introduce one or more site-specific modifications in a target polynucleotide sequence. In some aspects, a genome editing system comprises a Cas-alpha endonuclease, one or more guide polynucleotides, and a donor DNA. Some exemplary Cas-alpha endonucleases are described, for example, in U.S. Ser. No. 10/934,536 and WO2022082179.

A Cas-alpha endonuclease is a functional RNA-guided, PAM-dependent dsDNA cleavage protein of fewer than 800 amino acids, comprising: a C-terminal RuvC catalytic domain split into three subdomains and further comprising bridge-helix and one or more Zinc finger motif(s); and an N-terminal Rec subunit with a helical bundle, WED wedge-like (or “Oligonucleotide Binding Domain”, OBD) domain, and, optionally, a Zinc finger motif.

Cas-alpha endonucleases comprise one or more Zinc Finger (ZFN) coordination motif(s) that may form a Zinc binding domain. Zinc Finger-like motifs can aid in target and non-target strand separation and loading of the guide polynucleotide into the DNA target. Cas-alpha endonucleases comprising one or more Zinc Finger motifs can provide additional stability to a ribonucleoprotein complex on a target polynucleotide. Cas-alpha endonucleases comprise C4 or C3H zinc binding domains.

A Cas-alpha endonuclease can function as a double-strand-break-inducing agent, a single-strand-break inducing agent, or as a nickase. In some aspects, a catalytically inactive Cas-alpha endonuclease can be used to target or recruit to a target DNA sequence but not induce cleavage. In some aspects, a catalytically inactive Cas-alpha protein can be combined with a base editing molecule, such as a cytidine deaminase or an adenine deaminase.

### Cas9 Endonuclease

In some aspects of the methods disclosed herein, a genome editing system comprises a Cas9 endonuclease and one or more guide polynucleotides that introduce one or more site-specific modifications in a target polynucleotide sequence. In some aspects, a genome editing system comprises a Cas9 endonuclease, one or more guide polynucleotides, and a donor DNA. Some exemplary Cas9 endonucleases are described, for example, in WO2019165168.

Cas9 (formerly referred to as Cas5, Csn1, or Csx12) is a Cas endonuclease that forms a complex with a crNucleotide and a tracrNucleotide, or with a single guide polynucleotide, for specifically recognizing and cleaving all or part of a DNA target sequence. The canonical Cas9 recognizes a 3′ GC-rich PAM sequence on the target dsDNA, typically comprising an NGG motif. The Cas endonucleases described herein may recognize additional PAM sequences and used to modify target sites with different recognition sequence specificity.

A Cas9 polypeptide comprises a RuvC nuclease with an HNH (H—N—H) nuclease adjacent to the RuvC-II domain. The RuvC nuclease and HNH nuclease each can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al., 2013, Cell 157:1262-1278). Cas9 endonucleases are typically derived from a type II CRISPR system, which includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA (Makarova et al. 2015, Nature Reviews Microbiology Vol. 13:1-15).

The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target. In some aspects, a guide polynucleotide comprises a synthetic fusion of two RNA molecules, a crRNA (CRISPR RNA) comprising a variable targeting domain, and a tracrRNA. In some aspects, a guide polynucleotide comprises a variable targeting domain of 12 to 30 nucleotides and an RNA fragment that interacts with a Cas9 endonuclease.

### NHEJ and HDR

In some aspects of the methods described herein, a genome editing system comprises a Cas endonuclease, one or more guide polynucleotides, and optionally donor DNA, and editing a target polynucleotide sequence comprises nonhomologous end-joining (NHEJ) or homologous recombination (HR) following a Cas endonuclease-mediated double-strand break. Once a double-strand break is induced in the DNA, the cell's DNA repair mechanism is activated to repair the break. The most common repair mechanism to bring the broken ends together is the nonhomologous end-joining pathway (Bleuyard et al., (2006) DNA Repair 5:1-12). The structural integrity of chromosomes is typically preserved by the repair, but deletions, insertions, or other rearrangements are possible (Siebert and Puchta, (2002) Plant Cell 14:1121-31; Pacher et al., (2007) Genetics 175:21-9). Alternatively, the double-strand break can be repaired by homologous recombination between homologous DNA sequences. Once the sequence around the double-strand break is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

As used herein, “donor DNA” is a DNA construct that comprises a polynucleotide of interest to be inserted into the target site of a Cas endonuclease. Once a double-strand break is introduced in the target site by the endonuclease, the first and second regions of homology of the donor DNA can undergo homologous recombination with their corresponding genomic regions of homology resulting in exchange of DNA between the donor and the target genome. As such, the provided methods result in the integration of the polynucleotide of interest of the donor DNA into the double-strand break in the target site in the plant genome, thereby altering the original target site and producing an altered genomic target site.

In some aspects of the methods described herein, the Cas polypeptide has endonuclease activity. In some aspects, the Cas polypeptide is Cas12f or Cas9. In some aspects of methods for editing a plant genome, the method further comprises providing the plant cell with a donor DNA.

### Base Editing

In some aspects of the methods described herein, a genome editing system comprises a base editing agent and a plurality of guide polynucleotides and editing a target polynucleotide sequence comprises introducing a plurality of nucleobase edits in the target polynucleotide sequence resulting in a variant nucleotide sequence.

One or more nucleobases of a target polynucleotide can be chemically altered, in some cases to change the base from one type to another, for example from a Cytosine to a Thymine, or an Adenine to a Guanine. In some aspects, a plurality of bases, for example 2 or more, 5 or more, 10 or more, 20 or more, 30 or more, 40 or more, 50 or more, 60 or more, 70 or more, 80 or more 90 or more, 100 or more, or even greater than 100, 200 or more, up to thousands of bases may be modified or altered, to produce a plant with a plurality of modified bases.

Any base editing complex, such as a base editing agent associated with an RNA-guided protein, may be used to target and bind to a desired locus in the genome of an organism and chemically modify one or more components of a target polynucleotide.

Site-specific base conversions can be achieved to engineer one or more nucleotide changes to create one or more edits into the genome. These include for example, a site-specific base edit mediated by an C•G to T•A or an A•T to G•C base editing deaminase enzymes (Gaudelli et al., Programmable base editing of A•T to G•C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4. A catalytically “dead” or inactive Cas9 (dCas9), for example a catalytically inactive “dead” version of a Cas endonuclease disclosed herein, fused to a cytidine deaminase or an adenine deaminase protein becomes a specific base editor that can alter DNA bases without inducing a DNA break. Base editors convert C->T (or G->A on the opposite strand) or an adenine base editor that would convert adenine to inosine, resulting in an A->G change within an editing window specified by the gRNA. Any molecule that effects a change in a nucleobase is a “base editing agent”.

For many traits of interest, the creation of single double-strand breaks and the subsequent repair via HDR or NHEJ is not ideal for quantitative traits. An observed phenotype includes both genotype effects and environmental effects. The genotype effects further comprise additive effects, dominance effects, and epistatic effects. The probability of no effect per any single edit can be greater than zero, and any single phenotypic effect can be small, depending on the method used and site selected. Double-stranded break repair can additionally be “noisy” and have low repeatability.

One approach to ameliorate the probability of no effect per edit or small phenotypic effect outcome is to multiplex genome modification, such that a plurality of target sites are modified. Methods to modify a genomic sequence that do not introduce double-strand breaks would allow for single base substitutions. Combining these approaches, multiplexed base editing is beneficial for creating large numbers of genotype edits that can produce observable phenotype modifications. In some cases, dozens or hundreds or thousands of sites can be edited within one or a few generations of an organism.

A multiplexed approach to base editing in an organism, has the potential to create a plurality of significant phenotypic variations in one or a few generations, with a positive directional bias to the effects. In some aspects, the organism is a plant. A plant or a population of plants with a plurality of edits can be cross-bred to produce progeny plants, some of which will comprise multiple pluralities of edits from the parental lines. In this way, accelerated breeding of desired traits can be accomplished in parallel in one or a few generations, replacing time-consuming traditional sequential crossing and breeding across multiple generations.

A base editing deaminase, such as a cytidine deaminase or an adenine deaminase, may be fused to an RNA-guided endonuclease that can be deactivated (“dCas”, such as a deactivated Cas9) or partially active (“nCas”, such as a Cas9 nickase) so that it does not cleave a target site to which it is guided. The dCas forms a functional complex with a guide polynucleotide that shares homology with a polynucleotide sequence at the target site, and is further complexed with the deaminase molecule. The guided Cas endonuclease recognizes and binds to a double-stranded target sequence, opening the double-strand to expose individual bases. In the case of a cytidine deaminase, the deaminase deaminates the cytosine base and creates a uracil. Uracil glycosylase inhibitor (UGI) is provided to prevent the conversion of U back to C. DNA replication or repair mechanisms then convert the Uracil to a thymine (U to T), and subsequent repair of the opposing base (formerly G in the original G-C pair) to an Adenine, creating a T-A pair. For example, see Komor et al. Nature Volume 533, Pages 420-424, 19 May 2016.

In some aspects of the methods described herein, the Cas polypeptide comprises a deactivated Cas endonuclease (dCas) operably associated with a deaminase such as a cytosine deaminase or an adenine deaminase. In some aspects, the dCas polypeptide is dCas12f or dCas9.

### Prime Editing

In some aspects of the methods described herein, a genome editing system comprises a prime editing agent and a guide polynucleotide and editing a target nucleotide sequence comprises introducing one or more insertions, deletions, or nucleobase swaps in a target nucleotide sequence without generating a double-stranded DNA break.

In some aspects, the prime editing agent is a Cas polypeptide fused to a reverse transcriptase, wherein the Cas polypeptide is modified to nick DNA rather than generating double-strand break. This Cas-polypeptide-reverse transcriptase fusion can also be referred to as a “prime editor” or “PE”. In some aspects, the guide polynucleotide comprises a prime editing guide polynucleotide (pegRNA), and is larger than standard sgRNAs commonly used for CRISPR gene editing (e.g., >100 nucleobases). The pegRNA comprises a primer binding sequence (PBS) and a template containing the desired or target RNA sequence at its 3′ end.

During prime editing, the PE:pegRNA complex binds to a target DNA sequence and the modified Cas polypeptide nicks one target DNA strand resulting in a flap. The PBS on the pegRNA binds to the DNA flap and the target RNA sequence is reverse transcribed using the reverse transcriptase. The edited strand is incorporated into the target DNA at the end of the nicked flap, and the target DNA sequence is repaired with the new reverse transcribed DNA.

In some aspects of the methods described herein, the Cas polypeptide comprises a nickase Cas endonuclease (nCas) operably associated with a reverse transcriptase or co-expressed with a reverse transcriptase. In some aspects, the nCas polypeptide is nCas12f or nCas9.

In some aspects of the method, the at least one polynucleotide sequence encoding the at least one genome-editing component comprises a first polynucleotide sequence encoding a first genome-editing component and a second polynucleotide sequence encoding a second genome-editing component. In some aspects, the first genome-editing component is a Cas9 nuclease and the second genome-editing component is a guide RNA. In some aspects, the first genome-editing component is a Cas alpha nuclease and the second genome-editing component is a guide RNA. Exemplary Cas alpha nucleases are described in U.S. Pat. No. 10,934,536, incorporated herein in its entirety.

In yet another aspect, the disclosure provides a method of producing a doubled haploid plant, the method comprising: inducing somatic embryogenesis in a haploid embryo; transforming the haploid embryo with a polynucleotide sequence encoding a truncated ZM-ODP2 polypeptide; obtaining a somatic embryo or somatic embryogenic tissue expressing the polynucleotide sequence encoding the truncated ZM-ODP2 polypeptide; culturing the somatic embryo or somatic embryogenic tissue to obtain a plantlet, wherein a haploid set of chromosomes is diploidized; and regenerating a doubled haploid plant from the plantlet or a mature seed thereof.

In some aspects of the method, inducing somatic embryogenesis in the haploid embryo comprises transforming the haploid embryo with a morphogenic gene expression cassette comprising: (i) a polynucleotide sequence encoding a WUS/WOX polypeptide; (ii) a polynucleotide sequence encoding a ZM-ODP2 polypeptide; or a combination of (i) and (ii).

In some aspects of the method, the WUS/WOX polypeptide is selected from WUS1, WUS2, WUS3, WOX2A, WOX4, WOX5, and WOX9.

In some aspects of the method, the ZM-ODP2 polypeptide is selected from BBM2, BMN2, BMN3, and ODP2.

In some aspects of the method, the morphogenic gene expression cassette further comprises a PLTP promoter operably linked to the polynucleotide sequence encoding the WUS/WOX polypeptide. In some aspects, the morphogenic gene expression cassette further comprises an enhancer and/or an expression modulating element.

In some aspects of the method, transformation is mediated by Agrobacterium is selected from AGL-1, EHA105, GV3101, LBA4404, and LBA4404 THY−.

In some aspects of the method, diploidization is achieved without a chemical chromosome doubling agent.

In some aspects of the method, the polynucleotide sequence encoding the truncated ZM-ODP2 polypeptide is selected from: a polynucleotide sequence that has at least 85% sequence identify to SEQ ID NO: 1; a polynucleotide sequence that has at least 95% sequence identify to SEQ ID NO: 1; and a polynucleotide sequence that has SEQ ID NO: 1; and the truncated ZM-ODP2 polypeptide is selected from: a polypeptide sequence having at least 85% sequence identity to SEQ ID NO: 2; a polypeptide sequence having at least 95% sequence identity to SEQ ID NO: 2; and a polypeptide sequence having SEQ ID NO:2.

In a further aspect, the disclosure provides a method of producing a genome-edited doubled haploid plant, the method comprising: inducing somatic embryogenesis in a haploid embryo; transforming the haploid embryo with: (i) a polynucleotide sequence encoding a truncated ZM-ODP2 polypeptide; and (ii) at least one polynucleotide sequence encoding at least one genome-editing component; obtaining a somatic embryo or somatic embryogenic tissue expressing the polynucleotide sequence encoding the truncated ZM-ODP2 polypeptide and the at least one polynucleotide sequence encoding the at least one genome-editing component; culturing the somatic embryo or somatic embryogenic tissue to obtain a plantlet, wherein a haploid set of chromosomes is diploidized; and regenerating a doubled haploid plant from the plantlet or a mature seed thereof.

In some aspects of the method, inducing somatic embryogenesis in the haploid embryo comprises transforming the haploid embryo with a morphogenic gene expression cassette comprising: (i) a polynucleotide sequence encoding a WUS/WOX polypeptide; (ii) a polynucleotide sequence encoding a ZM-ODP2 polypeptide; or a combination of (i) and (ii).

In some aspects of the method, the WUS/WOX polypeptide is selected from WUS1, WUS2, WUS3, WOX2A, WOX4, WOX5, and WOX9.

In some aspects of the method, the ZM-ODP2 polypeptide is selected from BBM2, BMN2, BMN3, and ODP2.

In some aspects of the method, the morphogenic gene expression cassette further comprises a PLTP promoter operably linked to the polynucleotide sequence encoding the WUS/WOX polypeptide. In some aspects, the morphogenic gene expression cassette further comprises an enhancer and/or an expression modulating element.

In some aspects of the method, transformation is mediated by Agrobacterium is selected from AGL-1, EHA105, GV3101, LBA4404, and LBA4404 THY−.

In some aspects of the method, diploidization is achieved without a chemical chromosome doubling agent.

In some aspects of the method, the polynucleotide sequence encoding the truncated ZM-ODP2 polypeptide is selected from: a polynucleotide sequence that has at least 85% sequence identify to SEQ ID NO: 1; a polynucleotide sequence that has at least 95% sequence identify to SEQ ID NO: 1; and a polynucleotide sequence that has SEQ ID NO: 1; and the truncated ZM-ODP2 polypeptide is selected from: a polypeptide sequence having at least 85% sequence identity to SEQ ID NO: 2; a polypeptide sequence having at least 95% sequence identity to SEQ ID NO: 2; and a polypeptide sequence having SEQ ID NO:2.

In some aspects of the method, the at least one genome-editing component is a site-directed nuclease selected from meganucleases (MNs), zinc-finger nucleases (ZFN), transcription-activator like effector nucleases (TALENs), Cas9 nuclease, Cas alpha nuclease, Cpf1 nuclease, dCas9-FokI, dCpf1-FokI, chimeric Cas9-cytidine deaminase, chimeric Cas9 adenine deaminase, chimeric FEN1-Fok1, Mega-TALs, a nickase Cas9 (nCas9), chimeric dCas9 non-FokI nuclease, and dCpf1-non-FokI nuclease.

In some aspects of the method, the at least one polynucleotide sequence encoding the at least one genome-editing component comprises a first polynucleotide sequence encoding a first genome-editing component and a second polynucleotide sequence encoding a second genome-editing component. In some aspects, the first genome-editing component is a Cas9 nuclease and the second genome-editing component is a guide RNA. In some aspects, the first genome-editing component is a Cas alpha nuclease and the second genome-editing component is a guide RNA.

In a further aspect, the disclosure provides a method of seed sorting in doubled haploid plants, the method comprising: providing a diploid embryo of a plant with a polynucleotide sequence encoding at least a truncated ZM-ODP2 polypeptide; regenerating a T₀ plant from the diploid embryo, wherein the T₀ plant expresses the polynucleotide sequence encoding the truncated ZM-ODP2 polypeptide; obtaining a donor ear from the T₀ plant; pollinating the donor ear with pollen from a pollen donor plant; selecting a haploid embryo expressing the polynucleotide sequence encoding the truncated ZM-ODP2 polypeptide and lacking the genome of the haploid inducer plant, wherein the truncated ZM-ODP2 polypeptide promotes chromosome doubling of the haploid embryo to produce a doubled haploid embryo; regenerating a doubled haploid plant from the doubled haploid embryo or a mature seed thereof; and selecting a maternally-derived doubled haploid seed based on the absence of a paternal marker gene caused by parthenogenic haploid induction of a maternal egg cell.

In some aspects of the method, selecting the maternally-derived doubled haploid seed comprises using a manual method or an automated method.

In some aspects of the method, the automated method uses machine vision and/or machine learning methods.

In some aspects of the method, chromosome doubling is achieved without a chemical chromosome doubling agent.

In some aspects of the method, a second genetic chromosome doubling agent is provided to the diploid embryo of the plant along with the polynucleotide sequence encoding the truncated ZM-ODP2 polypeptide. In some aspects, the second genetic chromosome doubling agent comprises a polynucleotide sequence encoding a cyclin gene family member.

In some aspects of the method, the pollen donor plant is a non-haploid inducer plant.

In some aspects of the method, the pollen donor plant is a haploid inducer plant selected and/or derived from lines Stock 6, RWS, KEMS, KMS, or ZMS.

In some aspects of the method, the pollen donor plant comprises a paternal marker gene that is expressed in embryo tissue. In some aspects, the marker gene is a morphological marker, for example, a morphological marker expressing anthocyanin pigments. In some aspects, the marker gene is a reporter gene expressing a fluorescent protein, for example, GFP, YFP, CFP, or RFP.

In some aspects of the method, the polynucleotide sequence encoding the truncated ZM-ODP2 polypeptide is selected from: a polynucleotide sequence that has at least 85% sequence identify to SEQ ID NO: 1; a polynucleotide sequence that has at least 95% sequence identify to SEQ ID NO: 1; and a polynucleotide sequence that has SEQ ID NO: 1; and the truncated ZM-ODP2 polypeptide is selected from: a polypeptide sequence having at least 85% sequence identity to SEQ ID NO: 2; a polypeptide sequence having at least 95% sequence identity to SEQ ID NO: 2; and a polypeptide sequence having SEQ ID NO:2.

In a further aspect, the disclosure provides a method of producing a doubled haploid plant, the method comprising: providing a plant cell with a polynucleotide sequence encoding at least a truncated ZM-ODP2 polypeptide; regenerating a T₀ plant from the plant cell, wherein the T₀ plant expresses the polynucleotide sequence encoding the truncated ZM-ODP2 polypeptide; obtaining a donor ear from the T₀ plant; pollinating the donor ear with pollen from a pollen donor plant; expressing the polynucleotide sequence encoding the truncated ZM-ODP2 polypeptide, wherein the truncated ZM-ODP2 polypeptide promotes chromosome doubling of a haploid embryo to produce a doubled haploid embryo; selecting a doubled haploid embryo lacking the genome of the pollen donor plant; and regenerating a doubled haploid plant from the doubled haploid embryo or a mature seed thereof. In some aspects of the method, chromosome doubling is achieved without a chemical chromosome doubling agent. In some aspects of the method, a second genetic chromosome doubling agent is provided to the plant cell of the plant along with the polynucleotide sequence encoding the truncated ZM-ODP2 polypeptide. In some aspects of the method, the second genetic chromosome doubling agent comprises a polynucleotide sequence encoding a cyclin gene family member. In some aspects of the method, the pollen donor plant is a non-haploid inducer plant. In some aspects of the method, the pollen donor plant is a haploid inducer plant selected and/or derived from lines Stock 6, RWS, KEMS, KMS, or ZMS. In some aspects of the method, the pollen donor plant comprises a paternal marker gene that is expressed in embryo tissue. In some aspects of the method, the polynucleotide sequence encoding the truncated ZM-ODP2 polypeptide is selected from: (i) a polynucleotide sequence that has at least 85% sequence identify to SEQ ID NO: 1; (ii) a polynucleotide sequence that has at least 95% sequence identify to SEQ ID NO: 1; and (iii) a polynucleotide sequence that has SEQ ID NO: 1; and/or wherein the truncated ZM-ODP2 polypeptide is selected from: (i) a polypeptide sequence having at least 85% sequence identity to SEQ ID NO: 2; (ii) a polypeptide sequence having at least 95% sequence identity to SEQ ID NO: 2; and (iii) a polypeptide sequence having SEQ ID NO:2. In some aspects of the method, the truncated ZM-ODP2 polypeptide is part of a fusion protein that further comprises CBF1a, CBF3I, GNAT1, GNAT2, HAT1, HAT2, JMJ, VP16, or SV40:VP64. In some aspects of the method, the fusion protein comprises the truncated ZM-ODP2 polypeptide and CBF1a, and wherein the fusion protein comprises a polypeptide having at least 95% sequence identity to SEQ ID NO: 15.

In yet a further aspect, the disclosure provides a method of producing a genome-edited doubled haploid plant, the method comprising: providing a plant cell with a polynucleotide sequence encoding at least a truncated ZM-ODP2 polypeptide; and a polynucleotide sequence encoding a genome-editing component; regenerating a T₀ plant from the plant cell, wherein the T₀ plant expresses the polynucleotide sequence encoding at least the truncated ZM-ODP2 polypeptide and the polynucleotide sequence encoding the genome-editing component; obtaining a donor ear from the T₀ plant; pollinating the donor ear with pollen from a pollen donor; expressing the polynucleotide sequence encoding the truncated ZM-ODP2 polypeptide and the polynucleotide sequence encoding the genome-editing component, wherein the truncated ZM-ODP2 polypeptide promotes chromosome doubling of a haploid embryo to produce a doubled haploid embryo; selecting a doubled haploid embryo lacking the genome of the pollen donor plant; and regenerating a doubled haploid plant from the doubled haploid embryo or a mature seed thereof. In some aspects of the method, chromosome doubling is achieved without a chemical chromosome doubling agent. For example, a second genetic chromosome doubling agent is provided to the plant cell of the plant along with the polynucleotide sequence encoding the truncated ZM-ODP2 polypeptide. In some aspects of the method, the second genetic chromosome doubling agent comprises a polynucleotide sequence encoding a cyclin gene family member. In some aspects of the method, the pollen donor plant is a non-haploid inducer plant. In some aspects of the method, the pollen donor is a haploid inducer plant selected and/or derived from lines Stock 6, RWS, KEMS, KMS, or ZMS. In some aspects of the method, the pollen donor plant comprises a paternal marker gene that is expressed in embryo tissue. In some aspects of the method, the polynucleotide sequence encoding the truncated ZM-ODP2 polypeptide is selected from: (i) a polynucleotide sequence that has at least 85% sequence identify to SEQ ID NO: 1; (ii) a polynucleotide sequence that has at least 95% sequence identify to SEQ ID NO: 1; and (iii) a polynucleotide sequence that has SEQ ID NO: 1; and/or wherein the truncated ZM-ODP2 polypeptide is selected from: (i) a polypeptide sequence having at least 85% sequence identity to SEQ ID NO: 2; (ii) a polypeptide sequence having at least 95% sequence identity to SEQ ID NO: 2; and (iii) a polypeptide sequence having SEQ ID NO:2. In some aspects of the method, the truncated ZM-ODP2 polypeptide is part of a fusion protein that further comprises CBF1a, CBF3I, GNAT1, GNAT2, HAT1, HAT2, JMJ, or SV40:VP64. In some aspects of the method, the fusion protein comprises the truncated ZM-ODP2 polypeptide and CBF1a, and wherein the fusion protein comprises a polypeptide having at least 95% sequence identity to SEQ ID NO: 15. In some aspects of the method, the genome-editing component is a Cas9 nuclease or a Cas alpha nuclease, and the method further comprises providing the plant cell with a guide polynucleotide.

### DESCRIPTION OF THE FIGURES

FIG. 1 is a schematic diagram depicting a method of obtaining mature seed containing maternally derived doubled haploids in vivo.

FIG. 2A and FIG. 2B illustrates results of Example 3.

FIG. 3 is a schematic diagram depicting a method of obtaining mature, genome-modified seed containing maternally derived doubled haploids in vivo.

### DETAILED DESCRIPTION

The disclosures herein are described more fully hereinafter with reference to the accompanying figures, in which some, but not all possible aspects are shown. Indeed, disclosures may be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed methods and compositions pertain having the benefit of the teachings presented in the following descriptions and the associated figures. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of” Unless defined otherwise, all technical and scientific terms used herein have the same meaning as commonly understood by one of ordinary skill in the art to which the disclosed methods and compositions belong. In this specification and in the claims which follow, reference is made to a number of terms which shall be defined herein.

As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

All patents, publications and patent applications mentioned in the specification are indicative of the level of those skilled in the art to which this disclosure pertains. All patents, publications and patent applications are herein incorporated by reference in the entirety to the same extent as if each individual patent, publication or patent application was specifically and individually indicated to be incorporated by reference in its entirety. The following examples are offered by way of illustration and not by way of limitation.

In plants, germ line cells (germline) provide the transgenerational inheritance of genetic information in each subsequent generation by producing spore mother cells during sporogenesis. For example, sporogenesis provides the megaspore mother cell that develops the female gametes, the egg cell and central cell that give rise to the embryo and endosperm, respectively; or the microspore mother cell that develops the male gamete, giving rise to four haploid microspores, wherein each microspore further develops into a mature pollen grain. A key aspect for the unique role of germline cells is providing the genetic information a future offspring receives, wherein half of the genetic contribution is from the female gamete and half of the genetic contribution is from the male gamete. Fertilization of the egg cell with one sperm cell forms a diploid zygote, while a second sperm cells fuses with the two polar nuclei of the central cell to form a triploid endosperm. The endosperm is a terminally nourishing tissue for the embryo yet does not contribute to the germline. After fertilization, the zygote gives rise to an embryo, a process referred to as zygotic embryogenesis that is characteristic of sexual reproduction. A newly formed embryo undergoing such an embryogenesis developmental program comprising an underlying regulatory program affected by genetic determinants and epigenetic reprogramming leading from an embryogenic cell state to the acquisition of a differentiated cell fate, or cell fates, ultimately giving rise to a plant with all differentiated tissues thereof.

Cellular reprogramming refers to the method of providing a stimulus to alter the cell fate of a treated cell. Often cellular reprogramming comprises reverting a differentiated, more specialized cell towards an induced pluripotent stem cell state. Such methods can also comprise trans-differentiation, defined as the transformation of a cell other than a stem cell into a second cell type.

As used herein, “reprogram” or “reprograming” or “reprogramed” is a process of reverting or sensitizing mature, specialized cells into induced pluripotent stem cells or into cells in an embryonic/embryogenic state capable of being further developed into an embryo or embryo-like structure. In a population of cells that are being “reprogrammed” not all cells are expected to be “reprogrammed” to the same extent or to the same embryonic/embryogenic state. A mixture or mosaic of cells at various states of reprogramming is generally expected. Methods and compositions provided herein are expected to increase the ratio or percent of cells that are reprogrammed and in a desired embryonic/embryogenic state compared to cells that have not been exposed to the methods and compositions provided herein. Reprograming also refers to the re-establishment of germ cell development. Reprograming can occur when an embryogenesis inducing polypeptide is contacted with plant cells rendering the plant cells embryogenic. In some aspects, the methods of the present disclosure include contacting a haploid plant cell with an embryogenesis inducing agent such as a polypeptide to reprogram cell fate and cause the cell to become embryogenic. Alternatively, a polynucleotide encoding an embryogenesis inducing polypeptide may be introduced and expressed in a plant cell wherein the embryogenesis inducing polypeptide impacts surrounding/adjacent cells thereby rendering those surrounding/adjacent cells embryogenic. The cells may be reprogrammed in planta or ex situ.

As used herein, a “cellular reprogramming factor” or an “embryogenesis inducing agent” includes, but is not limited to, small molecules, compounds, embryogenesis factor gene products and morphogenic developmental gene embryogenesis inducing gene products that function in cell fate reprogramming either independently or in concert, including for example, microspore embryogenesis induction.

As used herein, a “cellular reprogramming treatment” is any of the treatments disclosed herein that elicits an embryogenesis response in the contacted cell.

The use of a cellular reprogramming agent (an embryogenesis inducing polypeptide or an embryogenesis inducing compound) or a cellular reprogramming treatment of a plant cell inside of the tissue of the organism, prior to cell isolation or cell extraction for experimentation and/or measurements done in an external environment is referred to as an “in planta” treatment or treatment method.

Cellular reprogramming affects cell fate and can result in various types of cell fate changes. One cell fate is a cell becoming totipotent, characterized as a cell that can form the sporophyte and extraembryonic cells, such as endosperm cells in the case of plants. Another cell fate is a cell becoming pluripotent, characterized as a cell that can give rise to all the cell types comprising the sporophyte, excluding extraembryonic cells. Embryogenic cells capable of direct organogenesis can be considered as pluripotent. Another cell fate is characterized as a cell becoming multipotent, defined as a cell that can develop into more than one cell type, but being more limited than pluripotent cells, such as plant cells undergoing indirect organogenesis. Reprogramming can also refer to the erasure of epigenetic marks characteristic of a differentiated, or a more specialized cell state and re-establishment of epigenetic marks characteristic of an embryogenic cell state.

Parthenogenesis is a natural form of asexual reproduction wherein growth and development of female gametes (embryos) occur without fertilization by sperm. The female gamete produced parthenogenetically may be either haploid or diploid.

Parthenogenesis induction refers to a method of providing a stimulus to a cell that improves levels of maternal haploid induction. Specifically, the gametes of a maize plant develop into a haploid plant when the plant is transformed with a genetic construct including regulatory elements and structural genes capable of altering the cellular fate of the plant cells. Further, the gametes of a maize plant can develop into a diploid plant when the plant is transformed with a genetic construct including regulatory elements and structural genes capable of altering cellular fate and cell cycle regulation of plant cells.

As used herein, a “parthenogenesis factor” or “PF” includes, but is not limited to, gene products that improve levels of maternal haploid induction and asexual reproduction wherein growth and development of female gametes (embryos) occur without fertilization by sperm when expressed in egg cells.

As used herein, a “parthenogenesis treatment” is any of the treatments disclosed herein that elicits a parthenogenic response in the contacted cell.

As used herein, “asexual reproduction” means reproduction without the fusion of gametes.

As used herein, “central cell” means the female gamete giving rise to the endosperm.

As used herein, “egg cell” means the female gamete giving rise to the embryo.

As used herein, “megaspore mother cell” means the cell that develops into the female gametophyte, also known as a megasporocyte, or functional megaspore (FMS).

As used herein, “microspore mother cell” means the cell that develops into the male gametophyte, also known as a microsporocyte.

As used herein, “gametogenesis” means the development of gametophytes from spores.

As used herein, “parthenogenesis” means the formation of an embryo from an unfertilized egg cell.

As used herein, “pseudogamy” means the fertilization-dependent formation of endosperm from a central cell.

As used herein, “sexual reproduction” means the mode of reproduction whereby female (egg) and male (sperm) gametes fuse to form a zygote.

As used herein, “somatic embryogenesis” means the formation of an embryo from a sporophytic cell without gamete and seed formation.

As used herein, “sporogenesis” means the formation of spores from spore mother cells.

As used herein, “spore mother cell” means the first cell of the reproductive lineage, formed from sporophytic cells in female and male reproductive tissues of the plant.

As used herein, “vegetative reproduction” means a form of reproduction in which a new plant is formed without the formation of an embryo.

As used herein, the term “embryo” means embryos and progeny of the same, immature and mature embryos, immature zygotic embryo, zygotic embryos, somatic embryos, embryogenic callus, and embryos derived from mature ear-derived seed. An embryo is a structure that is capable of germinating to form a plant.

As used herein, “haploid” means a plant or a plant cell having a single set (genome) of chromosomes and the reduced number of chromosomes (n) is equal to that in the gamete.

As used herein, the term “1n” or “1n cell” means a cell containing a single set of chromosomes, typically the product of meiosis. Examples of a In cell include gametes such as sperm cells, egg cells, or tissues derived from a gamete through mitotic divisions, such as a 1n embryo or a 1n plant. In maize where the plant is normally diploid, and the gametes are haploid, such gamete-derived embryos or plants are referred to as haploid embryos and haploid plants.

As used herein, “genetic chromosome doubling” refers to inducing chromosome doubling in a plant cell by providing the plant cell with one or more polynucleotides encoding a polypeptide, such as a polynucleotide encoding a truncated ZM-ODP2 polypeptide with or without a polynucleotide encoding a cyclin protein, that promotes, mediates, or modulates chromosome doubling to generate a doubled haploid cell or plant. More specifically, the methods described herein can achieve chromosome doubling in vivo and in vitro without the use of a chemical chromosome doubling agent, such as those detailed in Table 1.

TABLE 1
Chemical Chromosome Doubling Agents

| | | |
|---|---|---|
| Common Name/Trade | | |
| name | CAS | IUPAC |

Colchicine and Colchicine Derivatives

| | |
|---|---|
| colchicine/ | (S)-N-(5,6,7,9-tetrahydro-1,2,3,10- |
| acetyltrimethylcol- | tetramethoxy- 9-oxobenzo (a) heptalen- |
| chicinic acid | 7-yl) acetamide |
| colchicine derivatives | |

Carbamates

| | | |
|---|---|---|
| Carbetamide | (R)-1-(ethylcarbamoyl)ethyl | (2R)-N-ethyl-2- |
| | carbanilate | [[(phenylamino)carbonyl]oxy]pro- |
| | | panamide |
| chloropropham | | |
| Propham | | |

Benzamides

| | | |
|---|---|---|
| Pronamide/propyzamide | 3,5-dichloro-N-(1,1- | 3,5-dichloro-N-(1,1-dimethyl-2- |
| | dimethylpropynyl)ben-zamide | propynyl)benzamide |
| Tebutam | | |

Benzoic Acids

| | | |
|---|---|---|
| Chlorthal dimethyl | | |
| (DCPA), | | |
| Dicamba/dianat/ | 3,6-dichloro-o-anisic acid | 3,6-dichloro-2-methoxybenzoic acid |
| disugran (dicamba- | | |
| methyl) (BANVEL, | | |
| CLARITY) | | |

Dinitroaniline chromosome doubling agents

| | | |
|---|---|---|
| benfluralin/benefin/ | N-butyl-N-ethyl-α,α,α-trifluoro- | N-butyl-N-ethyl-2,6-dinitro-4- |
| (BALAN) | 2,6-dinitro-p-toluidine | (trifluoromethyl)benzenamine |
| Butralin | (RS)-N-sec-butyl-4-tert-butyl- | 4-(1,1-dimethylethyl)-N-(1- |
| | 2,6-dinitroaniline | methylpropyl)-2,6-dinitrobenzenamine |
| Chloralin | | |
| dinitramine | N1,N1-diethyl-2,6-dinitro-4- | N3,N3-diethyl-2,4-dinitro-6- |
| | trifluoromethyl-m- | (trifluoromethyl)-1,3-benzenediamine |
| | phenylenediamine | |
| ethalfluralin (Sonalan) | N-ethyl-α,α,α-trifluoro-N-(2- | N-ethyl-N-(2-methyl-2-propenyl)-2,6- |
| | methylallyl)-2,6-dinitro-p- | dinitro-4-(trifluoromethyl)benzenamine |
| | toluidine | |
| fluchloralin | N-(2-chloroethyl)-2,6-dinitro- | N-(2-chloroethyl)-2,6-dinitro-N-propyl- |
| | N-propyl-4- | 4-(trifluoromethyl)benzenamine |
| | (trifluoromethyl)aniline or | |
| | N-(2-chloroethyl)-α,α,α- | |
| | trifluoro-2,6-dinitro-N-propyl- | |
| | p-toluidine | |
| isopropalin | 4-isopropyl-2,6-dinitro-N,N- | 4-(1-methylethyl)-2,6-dinitro-N,N- |
| | dipropylaniline | dipropylbenzenamine |
| methalpropalin | α,α,α-trifluoro-N-(2- | N-(2-methyl-2-propenyl)-2,6-dinitro-N- |
| | methylallyl)-2,6-dinitro-N- | propyl-4-(trifluoromethyl)benzenamine |
| | propyl-p-toluidine | |
| nitralin | 4-methylsulfonyl-2,6-dinitro- | 4-(methylsulfonyl)-2,6-dinitro-N,N- |
| | N,N-dipropylaniline | dipropylbenzenamine |
| oryzalin (SURFLAN) | 3,5-dinitro-N4,N4- | 4-(dipropylamino)-3,5- |
| | dipropylsulfanilamide | dinitrobenzenesulfonamide |
| pendimethalin (PROWL) | N-(1-ethylpropyl)-2,6-dinitro- | N-(1-ethylpropyl)-3,4-dimethyl-2,6- |
| | 3,4-xylidine | dinitrobenzenamine |
| prodiamine | 5-dipropylamino-α,α,α- | 2,4-dinitro-N3,N3-dipropyl-6- |
| | trifluoro-4,6-dinitro-o-toluidine | (trifluoromethyl)-1,3-benzenediamine |
| | or 2,6-dinitro-N1,N1-dipropyl-4- | |
| | trifluoromethyl-m- | |
| | phenylenediamine | |
| profluralin | N-cyclopropylmethyl-α,α,α- | N-(cyclopropylmethyl)-2,6-dinitro-N- |
| | trifluoro-2,6-dinitro-N-propyl- | propyl-4-(trifluoromethyl)benzenamine |
| | p-toluidine or | |
| | N-cyclopropylmethyl-2,6- | |
| | dinitro-N-propyl-4- | |
| | trifluoromethylaniline | |
| trifluralin (TREFLAN, | α,α,α-trifluoro-2,6-dinitro-N,N- | 2,6-dinitro-N,N-dipropyl-4- |
| TRIFIC, TRILLIN) | dipropyl-p-toluidine | (trifluoromethyl)benzenamine |

Phosphoroamidates

| | | |
|---|---|---|
| APM (Amiprofos | | |
| methyl); amiprophos- | | |
| methyl | | |
| Butamifos | O-ethyl O-6-nitro-m-tolyl (RS)- | O-ethyl O-(5-methyl-2-nitrophenyl) (1- |
| | sec-butylphosphoramidothioate | methylpropyl)phosphoramidothioate |

Pyridines

| | | |
|---|---|---|
| Dithiopyr | | |
| Thiazopyr | methyl 2-difluoromethyl-5- | methyl 2-(difluoromethyl)-5-(4,5- |
| | (4,5-dihydro-1,3-thiazol-2-yl)- | dihydro-2-thiazolyl)-4-(2- |
| | 4-isobutyl-6- | methylpropyl)-6-(trifluoromethyl)-3- |
| | trifluoromethylnicotinate | pyridinecarboxylate |

As used herein, “diploid” means a plant or a plant cell having two sets (genomes) of chromosomes and the chromosome number (2n) is equal to that in the zygote.

As used herein, “diploid embryo” means an embryo having two sets (genomes) of chromosomes and the chromosome number (2n) is equal to that in the zygote.

As used herein, the term “2n” or “2n cell” means a cell containing two sets of chromosomes. Examples of 2n cells include a zygote, an embryo resulting from mitotic divisions of a zygote, or a plant produced by germination of a 2n embryo.

As used herein, “haploid plant” means a plant having a single set (genome) of chromosomes and the reduced number of chromosomes (n) is equal to that in the gamete.

As used herein, the term “diploid plant” means a plant having two sets (genomes) of chromosomes and the chromosome number (2n) is equal to that in the zygote.

As used herein, a “doubled haploid” or a “doubled haploid plant or cell” is one that is developed by the doubling of a haploid set of chromosomes, male or female. A plant or seed that is obtained from a doubled haploid (DH) plant that is any number of generations may still be identified as a doubled haploid plant. A doubled haploid plant is considered a homozygous plant. A plant is a doubled haploid if it is fertile, even if the entire vegetative part of the plant does not consist of the cells with the doubled set of chromosomes. For example, a plant is considered a doubled haploid plant if it contains viable gametes, even if it is chimeric. As used herein, “chromosome doubling” refers to a method resulting in a doubled haploid cell or plant from a haploid cell or plant. As used herein “diploidized” or “diploidization” refers to a cell that has undergone chromosome doubling to become a doubled haploid.

As used herein, a “doubled haploid embryo” is an embryo that has one or more cells containing 2 sets of homozygous chromosomes that can then be grown into a doubled haploid plant.

As used herein, the term “clonal” means multiple propagated plant cells or plants that are genetically, epigenetically and morphologically identical.

As used herein, the term “gamete” means a In reproductive cell such as a sperm cell, an egg cell or an ovule cell resulting from meiosis.

As used herein, the term “haploid embryo” means a gamete-derived somatic structure.

As used herein, the term “somatic structure” means a tissue, organ or organism.

As used herein, the term “somatic cell” is a cell that is not a gamete. Somatic cells, tissues or plants can be haploid, diploid, triploid, tetraploid, hexaploid, etc. A complete set of chromosomes is referred to as being 1n (haploid), with the number of chromosomes found in a single set of chromosomes being referred to as the monoploid number (x). For example, in the diploid plant Zea mays, 2n=2x=20 total chromosomes, while in diploid rice Oryza sativa, 2n=2x=24 total chromosomes. In a triploid plant, such as banana, 2n=3x=33 total chromosomes. In hexaploid wheat Triticum aestivum, 2n=6x=42. Ploidy levels can also vary between cultivars within the same species, such as in sugarcane, Saccharum officinarum, where 2n=10x=80 chromosomes, but commercial sugarcane varieties range from 100 to 130 chromosomes.

As used herein, the terms “modulate” or “mediate” refer to modifying, controlling, or stabilizing the expression or the strength of expression of a polynucleotide of interest including, but not limited to, up or down regulation.

As used herein, the term “modulator” refers to a polynucleotide that modifies, controls, or stabilizes the expression or the strength of expression of a polynucleotide of interest including, but not limited to, up or down regulation of the polynucleotide of interest.

As used herein, the term “medium” includes compounds in a liquid state, a gaseous state, or a solid state.

As used herein, the term “selectable marker” means a transgene that when expressed in a transformed/transfected cell confers resistance to selective agents such as antibiotics, herbicides and other compounds toxic to an untransformed/untransfected cell.

As used herein, the term “EAR” means an “Ethylene-responsive element binding factor-associated Amphiphilic Repression motif” with a general consensus sequence of LLxLxL, DNLxxP, LxLxPP, R/KLFGV, or TLLLFR that act as transcriptional repression signals within transcription factors. Addition of an EAR-type repressor element to a DNA-binding protein such as a transcription factor, dCAS9, or LEXA (as examples) confers transcriptional repression function to the fusion protein (Kagale, S., and Rozwadowski, K. 2010. Plant Signaling and Behavior 5:691-694).

As used herein, the term “transcription factor” means a protein that controls the rate of transcription of specific genes by binding to the DNA sequence of the promoter and either up-regulating or down-regulating expression. Examples of transcription factors, which are also morphogenic developmental genes, include members of the AP2/EREBP family (including the Babyboom (BBM) (also known as Ovule Development Protein 2 (ODP2)) genes and variants, plethora and aintegumenta sub-families, CAAT-box binding proteins such as LEC1 and HAP3, and members of the MYB, bHLH, NAC, MADS, bZIP and WRKY families.

As used herein, the term “synthetic transcription factor” refers to a molecule comprising at least two domains, a recognition domain and a regulatory domain not naturally occurring in nature.

As used herein, the term “expression cassette” means a distinct component of vector DNA consisting of coding and non-coding sequences including 5′ and 3′ regulatory sequences that control expression in a transformed/transfected cell.

As used herein, the term “coding sequence” means the portion of DNA sequence bounded by a start and a stop codon that encodes the amino acids of a protein.

As used herein, the term “non-coding sequence” means the portions of a DNA sequence that are transcribed to produce a messenger RNA, but that do not encode the amino acids of a protein, such as 5′ untranslated regions, introns and 3′ untranslated regions. Non-coding sequence can also refer to RNA molecules such as micro-RNAs, interfering RNA or RNA hairpins, that when expressed can down-regulate expression of an endogenous gene or another transgene.

As used herein, the term “regulatory sequence” means a segment of a nucleic acid molecule which is capable of increasing or decreasing the expression of a gene. Regulatory sequences include promoters, terminators, enhancer elements, silencing elements, 5′ UTR and 3′ UTR (untranslated region).

As used herein, the term “transfer cassette” means a T-DNA comprising an expression cassette or expression cassettes flanked by the right border and the left border.

As used herein, the term “T-DNA” means a portion of a Ti plasmid that is inserted into the genome of a host plant cell.

As used herein, the term “embryogenesis factor” means a gene that when expressed enhances improved formation of a somatically-derived structure. More precisely, ectopic expression of an embryogenesis factor stimulates de novo formation of an organogenic structure, for example a structure from embryogenic callus tissue, that can improve the formation of an embryo. This stimulated de novo embryogenic formation occurs either in the cell in which the embryogenesis factor is expressed, or in a neighboring cell. An embryogenesis factor gene can be a transcription factor that regulates expression of other genes or a gene that influences hormone levels in a plant cell which can stimulate embryogenic changes.

An embryogenesis factor is involved in plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof.

As used herein, the term “morphogenic developmental gene” or “morphogenic gene” means a gene that when ectopically expressed stimulates formation of a somatically-derived structure that can produce a plant. More precisely, ectopic expression of the morphogenic gene stimulates the de novo formation of a somatic embryo or an organogenic structure, such as a shoot meristem, that can produce a plant. This stimulated de novo formation occurs either in the cell in which the morphogenic gene is expressed, or in a neighboring cell. A morphogenic gene can be a transcription factor that regulates expression of other genes, or a gene that influences hormone levels in a plant tissue, both of which can stimulate morphogenic changes. A morphogenic gene may be stably incorporated into the genome of a plant or it may be transiently expressed. As used herein, the term “morphogenic factor” means a morphogenic gene and/or the protein expressed by a morphogenic gene. Some morphogenic developmental genes are parthenogenic.

Morphogenic genes involved in plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof, such as WUS/WOX genes (WUS1, WUS2, WUS3, WOX2A, WOX4, WOX5, or WOX9) see U.S. Pat. Nos. 7,348,468 and 7,256,322 and United States Patent Application publications 2017/0121722 and 2007/0271628; Laux et al. (1996) Development 122:87-96; and Mayer et al. (1998) Cell 95:805-815; van der Graaff et al., 2009, Genome Biology 10:248; Dolzblasz et al. 2016. Mol. Plant 19:1028-39 are useful in the methods of the disclosure. Modulation of WUS/WOX is expected to modulate plant and/or plant tissue phenotype including plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof. Expression of Arabidopsis WUS can induce stem cells in vegetative tissues, which can differentiate into somatic embryos (Zuo, et al. (2002) Plant J 30:349-359). Additional genes useful in the methods disclosed herein include, but are not limited to, a MYB118 gene (see U.S. Pat. No. 7,148,402), a MYB115 gene (see Wang et al. (2008) Cell Research 224-235), a BABYBOOM gene (BBM; see Boutilier et al. (2002) Plant Cell 14:1737-1749), or a CLAVATA gene (see, for example, U.S. Pat. No. 7,179,963). Morphogenic genes useful in the present disclosure include, but are not limited to, functional WUS/WOX genes.

Morphogenic polynucleotide sequences and amino acid sequences of WUS/WOX homeobox polypeptides can be used in the disclosed methods. As defined herein, a “functional WUS/WOX nucleotide” or a “functional WUS/WOX gene” is any polynucleotide encoding a protein that contains a homeobox DNA binding domain, a WUS box, and an EAR repressor domain (Ikeda et al., 2009 Plant Cell 21:3493-3505). As demonstrated by Rodriguez et al., 2016 PNAS www.pnas.org/cgi/doi/10.1073/pnas.1607673113 removal of the dimerization sequence which leaves behind the homeobox DNA binding domain, a WUS box, and an EAR repressor domain results in a functional WUS/WOX polypeptide. The WUSCHEL protein, designated hereafter as WUS, plays a key role in the initiation and maintenance of the apical meristem, which contains a pool of pluripotent stem cells (Endrizzi et al., (1996) Plant Journal 10:967-979; Laux, et al., (1996) Development 122:87-96; and Mayer, et al., (1998) Cell 95:805-815). Arabidopsis plants mutant for the WUS gene contain stem cells that are misspecified and that appear to undergo differentiation. WUS encodes a homeodomain protein which presumably functions as a transcriptional regulator (Mayer, et al., (1998) Cell 95:805-815). The stem cell population of Arabidopsis shoot meristems is believed to be maintained by a regulatory loop between the CLAVATA (CLV) genes which promote organ initiation and the WUS gene which is required for stem cell identity, with the CLV genes repressing WUS at the transcript level, and WUS expression being sufficient to induce meristem cell identity and the expression of the stem cell marker CLV3 (Brand, et al., (2000) Science 289:617-619; Schoof, et al., (2000) Cell 100:635-644). Constitutive expression of WUS in Arabidopsis has been shown to lead to adventitious shoot proliferation from leaves (in planta) (Laux, T., Talk Presented at the XVI International Botanical Congress Meeting, Aug. 1-7, 1999, St. Louis, Mo.).

In some aspects, the functional WUS/WOX homeobox polypeptide useful in the methods of the disclosure is a WUS1, WUS2, WUS3, WOX2A, WOX4, WOX5, WOX5A, or WOX9 polypeptide (see, U.S. Pat. Nos. 7,348,468 and 7,256,322 and US Patent Application Publication Numbers 2017/0121722 and 2007/0271628, herein incorporated by reference in their entirety and van der Graaff et al., 2009, Genome Biology 10:248). The functional WUS/WOX homeobox polypeptide useful in the methods of the disclosure can be obtained from or derived from any plant. Functional WUS/WOX nucleotides encoding proteins that contain a homeobox DNA binding domain, a WUS box, and an EAR repressor domain useful in the methods of the present disclosure are disclosed in US Patent Application Publication Number 2020/0270622 incorporated herein by reference in its entirety.

Other morphogenic genes useful in the present disclosure include, but are not limited to, LEC1 (U.S. Pat. No. 6,825,397 incorporated herein by reference in its entirety, Lotan et al., 1998, Cell 93:1195-1205), LEC2 (Stone et al., 2008, PNAS 105:3151-3156; Belide et al., 2013, Plant Cell Tiss. Organ Cult 113:543-553), KN1/STM (Sinha et al., 1993. Genes Dev 7:787-795), the IPT gene from Agrobacterium (Ebinuma and Komamine, 2001, In vitro Cell. Dev Biol—Plant 37:103-113), MONOPTEROS-DELTA (Ckurshumova et al., 2014, New Phytol. 204:556-566), the Agrobacterium AV-6b gene (Wabiko and Minemura 1996, Plant Physiol. 112:939-951), the combination of the Agrobacterium IAA-h and IAA-m genes (Endo et al., 2002, Plant Cell Rep., 20:923-928), the Arabidopsis SERK gene (Hecht et al., 2001, Plant Physiol. 127:803-816), the Arabidopsis AGL15 gene (Harding et al., 2003, Plant Physiol. 133:653-663), the FUSCA gene (Castle and Meinke, Plant Cell 6:25-41), and the PICKLE gene (Ogas et al., 1999, PNAS 96:13839-13844).

The present disclosure also includes plants obtained by any of the disclosed methods or compositions herein. The present disclosure also includes seeds from a plant obtained by any of the methods or compositions disclosed herein. As used herein, the term “plant” refers to whole plants, plant organs (e.g., leaves, stems, roots, etc.), plant tissues, plant cells, plant parts, seeds, propagules, embryos and progeny of the same. As used herein, the term “plant” refers to whole plants, plant organs (e.g., leaves, stems, roots, etc.), plant tissues, plant cells, plant parts, seeds, propagules, embryos and progeny of the same. Plant cells are differentiated or undifferentiated (e.g. callus, undifferentiated callus, immature and mature embryos, immature zygotic embryo, immature cotyledon, embryonic axis, suspension culture cells, protoplasts, leaf, leaf cells, root cells, phloem cells and pollen). Plant cells include, without limitation, cells from seeds, suspension cultures, explants, immature embryos, embryos, zygotic embryos, somatic embryos, embryogenic callus, meristem, somatic meristems, organogenic callus, protoplasts, embryos derived from mature ear-derived seed, leaf bases, leaves from mature plants, leaf tips, immature inflorescences, tassel, immature ear, silks, cotyledons, immature cotyledons, meristematic regions, callus tissue, cells from leaves, cells from stems, cells from roots, cells from shoots, gametophytes, sporophytes, pollen, microspores, multicellular structures (MCS), and embryo-like structures (ELS). Plant parts include differentiated and undifferentiated tissues including, but not limited to, roots, stems, shoots, leaves, pollen, seeds, tumor tissue and various forms of cells in culture (e.g., single cells, protoplasts, embryos, and callus tissue). The plant tissue may be in a plant or in a plant organ, tissue, or cell culture. Grain is intended to mean the mature seed produced by commercial growers for purposes other than growing or reproducing the species. Progeny, variants and mutants of the regenerated plants are also included within the scope of the disclosure, provided these progeny, variants and mutants are derived from regenerated plants made using the methods and compositions disclosed herein and/or comprise the introduced polynucleotides disclosed herein.

As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of a heterologous nucleic acid including those transgenics initially so altered as well as those created by sexual crosses or asexual propagation from the initial transgenic. A transgenic plant is defined as a mature, fertile plant that contains a transgene.

A transgenic “event” is produced by transformation of plant cells with a heterologous DNA construct, including a nucleic acid expression cassette that comprises a gene of interest, the regeneration of a population of plants resulting from the insertion of the transferred gene into the genome of the plant and selection of a plant characterized by insertion into a particular genome location. An event is characterized phenotypically by the expression of the inserted gene. At the genetic level, an event is part of the genetic makeup of a plant. The term “event” also refers to progeny produced by a sexual cross between the transformant and another plant wherein the progeny include the heterologous DNA.

The compositions and methods of the present disclosure are applicable to a broad range of plant species, including dicotyledonous plants and monocotyledonous plants. Representative examples of plants that are treated in accordance with the methods disclosed herein include, but are not limited to, wheat, cotton, sunflower, safflower, tobacco, Arabidopsis, barley, oats, rice, maize, triticale, sorghum, rye, millet, flax, sugarcane, banana, cassava, common bean, cowpea, tomato, potato, beet, grape, Eucalyptus, wheat grasses, turf grasses, alfalfa, clover, soybean, peanuts, citrus, papaya, Setaria sp, cacao, cucumber, apple, Capsicum, bamboo, melon, ornamentals including commercial garden and flower bulb species, fruit trees, vegetable species, Brassica species, as well as interspecies hybrids. In some aspects, the compositions and methods of the disclosure are applied to maize plants.

The methods of the disclosure involve introducing a polypeptide, polynucleotide (i.e., DNA or RNA), or nucleotide construct (i.e., DNA or RNA) into a plant. As used herein, “introducing” or “providing” means presenting to the plant the polynucleotide, polypeptide, or nucleotide construct in such a manner that the polynucleotide, polypeptide, or nucleotide construct gains access to the interior of a cell of the plant. The methods of the disclosure do not depend on a particular method for introducing the polynucleotide, polypeptide, or nucleotide construct into a plant, only that the polynucleotide, polypeptide, or nucleotide construct gains access to the interior of at least one cell of the plant. Methods for introducing polynucleotides, polypeptides, or nucleotide constructs into plants include, but are not limited to, stable transformation methods, transient transformation methods and virus-mediated methods.

As used herein, a “stable transformation” is a transformation in which the polynucleotide or nucleotide construct introduced into a plant integrates into the genome of the plant and is capable of being inherited by the progeny thereof. “Transient transformation” means that a polynucleotide or nucleotide construct is introduced into the plant and does not integrate into the genome of the plant or a polypeptide is introduced into a plant. In addition, “transient”, in certain embodiments may represent the presence of a parthenogenesis inducing agent in a cell where such an agent has been exogenously applied or secreted from a neighboring cell or is being produced from an extrachromosomal location (e.g., plasmid or another independently replicating origin), or not produced by a stably integrated recombinant DNA construct within the same cell.

As used herein, “contacting”, “comes in contact with” or “in contact with” mean “direct contact” or “indirect contact”. For example, cells are placed in a condition where the cells can come into contact with any of the parthenogenesis factors disclosed herein and/or an embryogenesis factor, a morphogenic developmental gene, a small molecule, or a doubling agent. Such substance is allowed to be present in an environment where the cells survive (for example, medium or expressed in the cell or expressed in an adjacent cell) and can act on the cells. For example, the medium comprising a doubling agent may have direct contact with the haploid cell or the medium comprising the doubling agent may be separated from the haploid cell by filter paper, plant tissues, or other cells thus the doubling agent is transferred through the filter paper or cells to the haploid cell.

As used herein, the term “biparental cross” is the cross-fertilization of two genetically different plants to obtain the first filial (F₁) generation of offspring and/or any successive filial generation thereafter. As used herein a biparental cross includes the offspring that are the progeny of any filial generation of offspring, including cross-fertilizing an offspring to one of its parental lines or an individual genetically like its parent to obtain progeny with a genetic identity closer to that of the parent referred to as a “backcross” and/or any successive backcross generation thereafter.

The methods provided herein rely upon the use of bacteria-mediated and/or biolistic-mediated gene transfer to produce regenerable plant cells. Bacterial strains useful in the methods of the disclosure include, but are not limited to, a disarmed Agrobacteria, an Ochrobactrum bacteria or a Rhizobiaceae bacteria (U.S. Pat. No. 9,365,859 incorporated herein by reference in its entirety). Standard protocols for particle bombardment (Finer and McMullen, 1991, In Vitro Cell Dev. Biol.—Plant 27:175-182), Agrobacterium-mediated transformation (Jia et al., 2015, Int J. Mol. Sci. 16:18552-18543; US2017/0121722 incorporated herein by reference in its entirety), or Ochrobactrum-mediated transformation (US2018/0216123 incorporated herein by reference in its entirety) can be used with the methods and compositions of the disclosure. Numerous methods for introducing heterologous genes into plants are known and can be used to insert a polynucleotide into a plant host, including biological and physical plant transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant and include chemical transfection methods such as calcium phosphate, microorganism-mediated gene transfer such as Agrobacterium (Horsch, et al., (1985) Science 227:1229-31), Ochrobactrum (US2018/0216123), electroporation, micro-injection and biolistic bombardment. Expression cassettes and vectors and in vitro culture methods for plant cell or tissue transformation and regeneration of transgenic plants are known and available. See, e.g., Gruber, et al., “Vectors for Plant Transformation,” in Methods in Plant Molecular Biology and Biotechnology, supra, pp. 89-119.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (Townsend, et al., U.S. Pat. No. 5,563,055 and Zhao, et al., U.S. Pat. No. 5,981,840), Ochrobactrum-mediated transformation (US2018/0216123), direct gene transfer (Paszkowski, et al., (1984) EMBO J. 3:2717-2722) and ballistic particle acceleration (see, for example, U.S. Pat. Nos. 4,945,050; 5,879,918; 5,886,244; 5,932,782; Tomes, et al., (1995) in Plant Cell, Tissue, and Organ Culture: Fundamental Methods, ed. Gamborg and Phillips (Springer-Verlag, Berlin); McCabe, et al., (1988) Biotechnology 6:923-926) and Lec1 transformation (WO 00/28058). See also, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Softener, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Ishida, et al., (1996) Nature Biotechnology 14:745-750 (maize via Agrobacterium tumefaciens), all of which are herein incorporated by reference in their entirety. Methods and compositions for rapid plant transformation are also found in U.S. 2017/0121722, herein incorporated in its entirety by reference. Vectors useful in plant transformation are found in U.S. patent application Ser. No. 15/765,521, herein incorporated by reference in its entirety.

Reporter genes or selectable marker genes may also be included in the expression cassettes of the present disclosure. Examples of suitable reporter genes are found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Other genes may be used the expression cassettes of the present disclosure that also assist in the recovery of transgenic events and include, but are not limited to, GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449), herein incorporated by reference in their entirety.

As used herein “recombinant” means a cell or vector, that has been modified by the introduction of a heterologous nucleic acid or a cell derived from a cell so modified. Thus, for example, a recombinant cell is a cell expressing a gene that is not found in identical form or location within the native (non-recombinant) cell or a cell that expresses a native gene in an expression pattern that is different from that of the native (non-recombinant) cell for example, the native gene is abnormally expressed, under expressed, has reduced expression or is not expressed at all because of deliberate human intervention. The term “recombinant” as used herein does not encompass the alteration of a cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette is incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “regulatory element” refers to a nucleic acid molecule having gene regulatory activity, i.e. one that has the ability to affect the transcriptional and/or translational expression pattern of an operably linked transcribable polynucleotide. The term “gene regulatory activity” thus refers to the ability to affect the expression of an operably linked transcribable polynucleotide molecule by affecting the transcription and/or translation of that operably linked transcribable polynucleotide molecule. Gene regulatory activity may be positive and/or negative and the effect may be characterized by its temporal, spatial, developmental, tissue, environmental, physiological, pathological, cell cycle, and/or chemically responsive qualities as well as by quantitative or qualitative indications.

In an aspect, a regulatory element expressed in the egg cell of the plant is useful for regulating ZM-ODP2 polypeptide activity to induce maternal haploid induction, resulting in a percentage of the progeny produced being haploid (having half the number of chromosomes compared to the parent). In addition, alternative regulatory elements are used to further optimize parthenogenic maternal haploid induction levels. For example, regulatory elements such as those disclosed in US2015/0152430 (promoters including, but not limited to the AT-DD5 promoter, the AT-DD31 promoter, the AT-DD65 promoter, and the ZM-DD45) and those disclosed in US2018/0094273 (Zea mays egg cell promoters) are used in the methods of the present disclosure (US2015/0152430 and US2018/0094273 incorporated herein by reference in their entireties).

Cis regulatory elements are regulatory elements that affect gene expression. Cis regulatory elements are regions of non-coding DNA that regulate the transcription of neighboring genes, often as DNA sequences in the vicinity of the genes that they regulate. Cis regulatory elements typically regulate gene transcription by encoding DNA sequences conferring transcription factor binding.

As used herein “promoter” is an exemplary regulatory element and generally refers to a nucleotide sequence capable of controlling the expression of a coding sequence or functional RNA. In general, a coding sequence is located 3′ to a promoter sequence. The promoter sequence comprises proximal and more distal upstream elements, the latter elements are often referred to as enhancers. Accordingly, an “enhancer” is a nucleotide sequence that can stimulate promoter activity and may be an innate element of the promoter or a heterologous element inserted to enhance the level or tissue-specificity of a promoter. Promoters may be derived in their entirety from a native gene or may be composed of different elements derived from different promoters found in nature, or even comprise synthetic nucleotide segments. Different regulatory elements may direct the expression of a gene in different tissues or cell types, or at different stages of development, or in response to different environmental conditions.

A “plant promoter” is a promoter capable of initiating transcription in plant cells. Exemplary plant promoters include, but are not limited to, those that are obtained from plants, plant viruses, and bacteria such as Agrobacterium or Rhizobium, which comprise genes expressed in plant cells. Examples are promoters that preferentially initiate transcription in certain tissues, such as leaves, roots, seeds, fibers, xylem vessels, tracheids or sclerenchyma. Such promoters are referred to as “tissue preferred” promoters. A “cell type” specific promoter primarily drives expression in certain cell types in one or more organs, for example, vascular cells in roots or leaves. An “inducible” or “regulatable” promoter is a promoter which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters are members of the class of “non-constitutive” promoters. A “constitutive” promoter is a promoter that causes a nucleic acid fragment to be expressed in most cell types at most times under most environmental conditions and states of development or cell differentiation.

In an aspect, egg cell promoters and egg cell specific promoters are useful in the methods of the present disclosure. In addition to those egg cell promoters and/or egg cell specific promoters disclosed herein and those disclosed in US2015/0152430 and US2018/0094273, each of which is incorporated herein in its entirety, egg cell promoters and/or egg cell specific promoters useful in the present disclosure include, but are not limited to the egg cell-specific EC1.1 and EC1.2 promoters disclosed in Sprunck et al., (2012) Science, 338, 1093-1097 and Steffen et al., (2007) Plant J., 51:281-92.

A “translation leader sequence” refers to a nucleotide sequence located between the promoter sequence of a gene and the coding sequence. The translation leader sequence is present in the fully processed mRNA upstream of the translation start sequence. The translation leader sequence may affect numerous parameters including, processing of the primary transcript to mRNA, mRNA stability and/or translation efficiency. Examples of translation leader sequences have been described (Turner and Foster (1995) Mol. Biotechnol. 3:225-236).

As used herein, “heterologous” refers to a nucleic acid that originates from a foreign species, or, if from the same species, is substantially modified from its native form in composition and/or genomic locus by deliberate human intervention. For example, a promoter operably linked to a heterologous structural gene that is from a species different from that from which the structural gene was derived, or, if from the same species, one or both are substantially modified from their original form and/or genomic location.

The polynucleotide encoding a truncated BBM useful in the methods of the disclosure, and optionally a morphogenic developmental gene sequence, can be provided in expression cassettes for expression in a plant of interest. The cassette can include 5′ and 3′ regulatory sequences operably linked to a polynucleotide encoding a truncated BBM and optionally morphogenic developmental gene sequence disclosed herein. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions (fusion proteins), by operably linked it is intended that the coding regions are in the same reading frame. The cassette may additionally contain at least one additional gene to be co-transformed into the organism. Alternatively, polynucleotide encoding a truncated BBM and optional morphogenic developmental gene(s) are provided on multiple expression cassettes. Such an expression cassette is provided with a plurality of restriction sites for insertion under the transcriptional regulation of the regulatory regions (i.e., promoter(s)). The expression cassette may additionally contain selectable marker genes.

### A. Polynucleotides and Polypeptides

The present disclosure provides methods of inducing parthenogenic haploid induction and genetic chromosome doubling in vivo in a plant cell using a truncated morphogenic developmental gene. Also disclosed are methods of inducing somatic embryogenesis and genetic chromosome doubling in vitro using the truncated morphogenic developmental gene.

Polynucleotides useful in the methods of the disclosure include the ZM-ODP (TR5) morphogenic developmental gene, which encodes a truncated maize Ovule Development Protein 2 (ZM-ODP2), also referred to herein as BBM⁴⁰⁴. The ZM-ODP2 polynucleotide sequence can be operably linked to an inducible promoter, a tissue-preferred promoter, or a promoter that is both inducible and tissue-preferred. For example, a promoter that can be both haploid-tissue specific and inducible.

In some aspects, the methods use the ZM-ODP2 (TR5) morphogenic developmental gene polynucleotide (SEQ ID NO: 1) encoding the ZM-ODP2-(266-669) polypeptide (SEQ ID: 2).

In some aspects, the methods use a polynucleotide sequence having at least 85% sequence identity to SEQ ID NO: 1.

In some aspects, the methods use a polynucleotide sequence having at least 90% sequence identify to SEQ ID NO: 1.

In some aspects, the methods use a polynucleotide sequence having at least 95% sequence identify to SEQ ID NO: 1.

In some aspects, the methods use a polynucleotide sequence having at least 85%, 86%, 87%, 88%, 89%, 90%, 91%, 92%, 93%, 94%, 95%, 96%, 97%, 98%, 99%, 99.5% or 99.9% sequence identity to SEQ ID NO: 1.

In some aspects, the methods use a polynucleotide sequence having SEQ ID NO: 1.

In some aspects, the methods use the ZM-ODP2-(266-669) polypeptide having at least 85% sequence identity to SEQ ID NO: 2.

In some aspects, the methods use the ZM-ODP2-(266-669) polypeptide having at least 90% sequence identity to SEQ ID NO: 2.

In some aspects, the methods use the ZM-ODP2-(266-669) polypeptide having at least 95% sequence identity to SEQ ID NO: 2.

In some aspects, the methods use the ZM-ODP2-(266-669) polypeptide having at least 85%, 86%, 87%, 88%, 89%, 90%, 91%, 92%, 93%, 94%, 95%, 96%, 97%, 98%, 99%, 99.5% or 99.9% sequence identity to SEQ ID NO: 2.

In some aspects, the methods use ZM-ODP2-(266-669) polypeptide having SEQ ID NO: 2.

In some aspects, the methods use a ZM-ODP2(TR5)-V1:AT-CBF1A polynucleotide (SEQ ID NO: 14) encoding a ZM-ODP2-(266-668):At-CBF1a fusion protein (SEQ ID: 15).

In some aspects, the methods use a polynucleotide sequence having at least 85% sequence identity to SEQ ID NO: 14.

In some aspects, the methods use a polynucleotide sequence having at least 90% sequence identify to SEQ ID NO: 14.

In some aspects, the methods use a polynucleotide sequence having at least 95% sequence identify to SEQ ID NO: 14.

In some aspects, the methods use a polynucleotide sequence having at least 85%, 86%, 87%, 88%, 89%, 90%, 91%, 92%, 93%, 94%, 95%, 96%, 97%, 98%, 99%, 99.5% or 99.9% sequence identity to SEQ ID NO: 14.

In some aspects, the methods use a polynucleotide sequence having SEQ ID NO: 14.

In some aspects, the methods use the ZM-ODP2-(266-668):At-CBF1a fusion protein having at least 85% sequence identity to SEQ ID NO: 15.

In some aspects, the methods use the ZM-ODP2-(266-668):At-CBF1a fusion protein having at least 90% sequence identity to SEQ ID NO: 15.

In some aspects, the methods use the ZM-ODP2-(266-668):At-CBF1a fusion protein having at least 95% sequence identity to SEQ ID NO: 15.

In some aspects, the methods use the ZM-ODP2-(266-668):At-CBF1a fusion protein having at least 85%, 86%, 87%, 88%, 89%, 90%, 91%, 92%, 93%, 94%, 95%, 96%, 97%, 98%, 99%, 99.5% or 99.9% sequence identity to SEQ ID NO: 15.

In some aspects, the methods use the ZM-ODP2-(266-668):At-CBF1a fusion protein having SEQ ID NO: 15.

In some aspects, the methods can utilize a secondary morphogenic developmental gene polynucleotide in addition to ZM-ODP2 (TR5), such as, WUS/WOX genes and other BBM (ODP2) genes and variants.

In some aspects, the methods can utilize a parthenogenesis factor (PF) in addition to ZM-ODP2 (TR5).

In some aspects, the methods can utilize a cell cycle gene in addition to ZM-ODP2 (TR5), including Cyclin A, Cyclin B, Cyclin C, Cyclin D, Cyclin E, Cyclin F, Cyclin G, and Cyclin H; Pin1; E2F; Cdc25; RepA genes and similar plant viral polynucleotides encoding replication-associated proteins (see U.S. Patent Publication No. 2002/0188965 incorporated herein by reference in its entirety).

In some aspects, the methods can use a secondary genetic chromosome doubling agent, wherein a cyclin gene is provided as a second genetic chromosome doubling agent. For example, a polypeptide encoding a truncated ZM-ODP2 polypeptide and be co-expressed with a polynucleotide containing a ZM-CYCD2 sequence (DNA SEQ ID NO: 49, Protein SEQ ID NO: 50).

In some aspects, the methods use a cell non-autonomous fusion polypeptide comprising a polynucleotide containing a ZM-ODP2 (TR5)-V1 sequence (SEQ ID NO: 8), a linker sequence (SEQ ID NO: 10), and a cell penetrating polypeptide (CPP) sequence, for example a KNOTTED-1 CPP DNA fragment (DNA SEQ ID NO: 38; Protein SEQ ID NO: 39) encoding a ZM-ODP2-(266-669):KNOTTED-1 CPP fusion polypeptide (DNA SEQ ID NO: 40; Protein SEQ ID NO: 41). In another aspect, it is understood that an alternative CPP sequence, either natural or synthetic variants, known in the art can be used to provide cell non-autonomous activity to such a fusion polypeptide.

In some aspects, the methods utilize a translational fusion protein comprising a heterologous, synthetic transcription factor. For example, a ZM-ODP2 (TR5) polynucleotide encoding a truncated ZM-ODP2 polypeptide can be fused to a maize optimized DNA sequence, AT-CBF1A (MO) encoding a C-repeat/DRE binding factor domain, referred to herein as “At-CBF1a”. Together, this synthetic transcription factor coding sequence encodes a truncated ZM-ODP2:At-CBF1a fusion protein. In some aspects, the ZM-ODP2:At-CBF1a fusion protein exhibits cell non-autonomous activity.

Additional domains suitable for use in a translational fusion protein are shown in Table 2. In some aspects, these synthetic transcription factors can modulate or improve the regulation of gene products conferring parthenogenic haploid induction and genetic chromosome doubling activities such as by creating and/or maintaining an open chromatin structure or improving assembly of the preinitiation complex. In some aspects, the fusion proteins described herein exhibit cell non-autonomous activity.

TABLE 2
Fusion Protein Domains

| | | DNA SEQ | PROTEIN SEQ |
|---|---|---|---|
| Domain | Description | ID NO | ID NO |
| GNAT1 | chromatin modifying histone | 26 | 32 |
| | acetyltransferase domain | | |
| GNAT2 | chromatin modifying histone | 27 | 33 |
| | acetyltransferase domain | | |
| HAT1 | chromatin modifying histone | 28 | 34 |
| | acetyltransferase domain | | |
| HAT2 | chromatin modifying histone | 29 | 35 |
| | acetyltransferase domain | | |
| JMJ | chromatin modifying lysine | 30 | 36 |
| | demethylase domain | | |
| SV40:VP64 | nuclear localization and | 31 | 37 |
| | viral transcriptional | | |
| | activator domain | | |

### B. Methods

In the methods of the present disclosure, the ZM-ODP2 (TR5) polynucleotide encoding the ZM-ODP2-(266-669) polypeptide (also referred herein as a “truncated ZM-ODP2 polypeptide”) are used for altering plant cell fate and ploidy levels in vivo. In some aspects, the polynucleotides and polypeptides can be used in combination with a secondary morphogenic developmental polynucleotide and/or a cell cycle gene.

In a first aspect, the disclosure provides a method for producing maternal haploid plants expressing a truncated ZM-ODP2 polypeptide resulting in an increased percentage of maternal haploids.

In another aspect, the disclosure provides methods for producing plants using asexual reproduction. Apogamy, a type of reproduction of flowering plants, is characterized by a diploid cell in the embryo sac developing into an embryo without being fertilized. Parthenogenesis is one form of apogamy and in a broader sense can include de novo embryogenic formation from a haploid gametophytic cell, for example an egg cell resulting from megasporogenesis.

In another aspect, the disclosure provides methods of transforming a plant cell with a bacterial strain containing a plasmid that comprises a transfer-DNA containing a ZM-ODP2 (TR5) polynucleotide.

In another aspect, the disclosure provides efficient and effective methods of producing populations of recombinant inbred lines including, but not limited to, methods of initiating inducing parthenogenic haploid induction and genetic chromosome doubling in plant cells using a ZM-ODP2 (TR5) polynucleotide and/or a truncated ZM-ODP2 polypeptide to enable generation of doubled haploid recombinant populations. In some aspects, a method of producing a doubled haploid plant from gametes by contacting a plant cell with a truncated ZM-ODP2 polypeptide that can activate haploid induction and genetic chromosome doubling. It will be understood that genetic chromosome doubling methods described herein are achieved without providing a chromosome doubling agent, for example a chemical chromosome doubling agent of Table 1, to a plant cell.

In another aspect, the disclosure provides methods of simultaneous parthenogenic haploid induction and in vivo chromosome doubling of an unfertilized egg in response to a providing a single protein (e.g., the ZM-ODP2-(266-669) polypeptide) to the unfertilized egg cell. In some aspects, the in vivo genetic chromosome doubling methods described herein resulted in chromosome doubling rates greater than baseline levels of spontaneous chromosomal doubling. For most maize germplasm, the rate of spontaneous chromosomal doubling is too low for reliable doubled haploid production, with an average frequency of approximately 1:900 (0.11%) with a range between 0-0.68% fertile haploid plants (see Chase, S. 1949. Genetics 34:328-32). Kleiber et al. reported a median fertile haploid level of 0.41% and the mean number of such plants producing mature, intact seed was 0.15% (see Kleiber et al. 2012. Crop Sci. 52:623-630).

One benefit of the genetic chromosome doubling methods disclosed herein is that use of artificial or chemical chromosome doubling agents or treatments can be rendered unnecessary. This effectively reduces usage and exposure to potentially harmful chemicals and simplifies the logistics of doubled haploid production. For example, the labor and time required for haploid embryo rescue is eliminated, or at least reduced, as are the in vitro tissue culturing steps, the transfer steps, and transplanting steps; as well as the attrition that can occur during each of these steps despite the investment and cost of the operating process.

In a further aspect, the disclosure provides methods of transforming a non-haploid inducer line to provide parthenogenic haploid induction, genetic chromosome doubling, and optionally genome modification to an unfertilized egg cell to obtain a fertile, doubled haploid plant optionally having a genome modification. The methods are performed in the absence of providing an artificial, chemical chromosome doubling agent to a maternally derived embryo. Further, these methods allow for the harvesting of intact, mature seed and do not require the use of immature haploid embryo rescue and associated in vitro tissue culture, currently used in the art.

In a further aspect, the disclosure provides a method of producing a doubled haploid plant from a plant cell. In some aspects, the method comprises (a) providing a diploid embryo of a plant with a polynucleotide sequence encoding a truncated ZM-ODP2 polypeptide; (b) regenerating a T₀ plant from the diploid embryo, wherein the T₀ plant expresses the polynucleotide sequence encoding the truncated ZM-ODP2 polypeptide; (c) obtaining a donor ear from the T₀ plant; (d) pollinating the donor ear with pollen from a pollen donor plant; (e) selecting a haploid embryo expressing the polynucleotide sequence encoding the truncated ZM-ODP2 polypeptide and lacking the genome of the haploid inducer plant, wherein the truncated ZM-ODP2 polypeptide promotes chromosome doubling of the haploid embryo to produce a doubled haploid embryo; and (f) regenerating a doubled haploid plant from the doubled haploid embryo or a mature seed thereof.

In some aspects, the methods described herein can utilize non-haploid inducer plants as pollen donors.

In some aspects, the methods described herein can utilize haploid inducer plants as pollen donors. Exemplary maize haploid inducer lines of the disclosure include, for example, Stock 6 (Coe, 1959, Am. Nat. 93:381 382; Sharkar and Coe, 1966, Genetics 54:453 464) RWS, KEMS (Deimling, Roeber, and Geiger, 1997, Vortr. Pflanzenzuchtg 38:203 224), or KMS and ZMS (Chalyk, Bylich & Chebotar, 1994, MNL 68:47; Chalyk & Chebotar, 2000, Plant Breeding 119:363 364), and indeterminate gametophyte (ig) mutation (Kermicle 1969 Science 166:1422 1424).

In some aspects, the pollen donor plant comprises a paternal marker gene that is expressed in embryo tissue such as a morphological marker or a reporter gene. In some aspects, the morphological marker expresses anthocyanin pigments. In some aspects, the reporter gene expresses a fluorescent protein (e.g., GFP, YFP, CFP, and RFP).

In some aspects, the methods described herein use pollen grains collected from the anthers of male haploid inducer plants or non-haploid inducer plants.

In some aspects, pollen donor plants can constitutively express a paternal color marker, such as a yellow fluorescent protein color marker (YFP), an anthocyanin color marker, or both. It is understood that the paternal color marker can comprise methods using alternative reporter gene activities, including but is not limited to detection methods wherein said paternal reporter gene product has embryo-preferred tissue specificity that can be detected using either manual or automated identification methods, or a combination thereof.

In some aspects, the pollen donor plant comprises a paternal marker gene, for example, a morphological marker expressing anthocyanin pigments or a reporter gene. In some aspects, the reporter gene expresses a fluorescent protein, for example, GFP, YFP, CFP, or RFP.

As detailed above, the method stimulates in vivo parthenogenic haploid induction and genetic chromosome doubling from expression of a single polypeptide. In another aspect, the disclosure provides a method of producing a doubled haploid plant comprising: (a) stimulating parthenogenic haploid induction and chromosome doubling by providing a haploid plant cell with a polynucleotide sequence encoding a truncated BBM polypeptide; (b) regenerating a T₀ plant expressing the polynucleotide sequence encoding the truncated BBM polypeptide, wherein a haploid set of chromosomes is diploidized; (c) pollinating the T₀ plant; (d) obtaining a doubled haploid embryo from the T₀ plant; and (e) regenerating a doubled haploid plant from the doubled haploid embryo or a mature seed thereof. As detailed above, the method stimulates in vivo parthenogenic haploid induction and genetic chromosome doubling from expression of a single polypeptide. The T0 plant can be pollinated via self-pollination or with pollen from a sister plant.

In some aspects, the methods of producing a doubled haploid plant utilize a translational fusion protein comprising a heterologous, synthetic transcription factor. For example, a ZM-ODP2 (TR5) polynucleotide encoding a truncated ZM-ODP2 polypeptide can be fused to a maize optimized DNA sequence, AT-CBF1A (MO) encoding a C-repeat/DRE binding factor domain, referred to herein as “At-CBF1a”. Together, this synthetic transcription factor coding sequence encodes a truncated ZM-ODP2:At-CBF1a fusion protein. In some aspects, providing a ZM-ODP2-(266-668):At-CBF1a fusion protein to an unfertilized egg cell improves parthenogenic haploid induction and genetic chromosome doubling responses in vivo, increases the proportion of regenerated plants that are diploidized, and/or increases the proportion of fertile regenerated plants.

In some aspects, a polynucleotide containing ZM-ODP2 (TR5)-V1 (SEQ ID NO: 8) that encodes a ZM-ODP2-(266-668) polypeptide (SEQ ID NO: 9) is fused with a linker sequence (DNA SEQ ID NO: 10; Protein SEQ ID NO: 11) which in turn is fused to a maize optimized DNA sequence, AT-CBF1A (MO) (SEQ ID NO: 12) that encodes At-CBF1a (SEQ ID NO: 13). This synthetic transcription factor coding sequence (SEQ ID NO: 14) encodes a ZM-ODP2-(266-668):At-CBF1a fusion protein (SEQ ID NO: 15) that is operably linked to a PvEC1 promoter (SEQ ID NO: 6).

An unexpected finding demonstrated herein is that a portion of null T1 plants produced mature, intact seed in response to self-pollination (see Example 3). This result is likely due to the ZM-ODP2-(266-669) polypeptide acting as a morphogen with a concentration gradient affecting adjacent cells. More specifically, the ZM-ODP2-(266-669) polypeptide expressed by a sporophytic, diploid (2n) cell hemizygous for the T-DNA of RV027603 acted as a morphogen affecting maternal cells, such as the embryo sac, the unfertilized egg cell, and/or an unfertilized haploid egg cell lacking the T-DNA of RV027603. In this manner, a concentration gradient formed an embryogenic condition sufficient to induce both haploid induction and in vivo chromosome doubling within a null-segregant egg cell such that chromosome doubling and fertility was sufficiently restored in 3.2% of the null T1 plants regenerated from the embryos contacted with the ZM-ODP2-(266-669) polypeptide. This level of restored fertility to a null embryo was greater than expected in response to spontaneous chromosomal doubling, and thus, demonstrated a novel improvement to the state of the art. In some aspects, the methods disclosed herein enable cell non-autonomous gene activity of the truncated ZM-ODP2 polypeptide by providing to a cell a ZM-ODP2-(266-669) fusion polypeptide that functions as a signaling molecule, is involved in synthesizing a signaling molecule, or participates in activating a signal transduction response. In this respect, the disclosure provides methods to obtain a genotypically wild type embryo with elevated parthenogenic haploid induction and in vivo genetic doubling activities in response to being contacted with a cell non-autonomous gene product (e.g., the truncated ZM-ODP2 polypeptide) provided from a non-embryo cell.

In some aspects, the cell non-autonomous methods of the disclosure form a concentration gradient providing an embryogenic condition sufficient to induce both haploid induction and in vivo chromosome doubling within a null-segregant egg cell. In some aspects, a T0 female parent plant having a F1 hybrid genome can produce intact, mature DO seed with proportions greater than that associated with spontaneous chromosomal doubling. The simultaneous parthenogenic haploid induction and in vivo chromosome doubling of an unfertilized egg cell can therefore provide a reliable method for producing fertile, non-transgenic maize doubled haploids grown from intact, mature seed.

In some aspects, a cell non-autonomous fusion polypeptide comprises a polynucleotide containing a ZM-ODP2 (TR5)-V1 sequence (SEQ ID NO: 8), a linker sequence (SEQ ID NO: 10), and a cell penetrating polypeptide (CPP) sequence, for example a KNOTTED-1 CPP DNA fragment (DNA SEQ ID NO: 38; Protein SEQ ID NO: 39) encoding a ZM-ODP2-(266-668):KNOTTED-1 CPP fusion polypeptide (DNA SEQ ID NO: 40; Protein SEQ ID NO: 41). In another aspect, it is understood that an alternative CPP sequence, either natural or synthetic variants, known in the art can be used to provide cell non-autonomous activity to such a fusion polypeptide.

In some aspects, a polynucleotide containing ZM-ODP2 (TR5)-V1 (SEQ ID NO: 8) that encodes a ZM-ODP2-(266-668) polypeptide (SEQ ID NO: 9) is fused with a linker sequence (DNA SEQ ID NO: 10; Protein SEQ ID NO: 11) which in turn is fused to a maize optimized DNA sequence, AT-CBF1A (MO) (SEQ ID NO: 12) that encodes At-CBF1a (SEQ ID NO: 13). This synthetic transcription factor coding sequence (SEQ ID NO: 14) encodes a ZM-ODP2-(266-668):At-CBF1a fusion protein (SEQ ID NO: 15) that is operably linked to a PvEC1 promoter (SEQ ID NO: 6). In some aspects, the ZM-ODP2-(266-668):At-CBF1a fusion protein exhibits cell non-autonomous activity.

In some aspects, to provide a fusion polypeptide as a solute from a non-reduced, diploid sporophytic cell hemizygous for the T-DNA containing an expression cassette to a reduced, haploid gametic cell, the fusion polypeptide can be expressed in the transfer cells located in the basal endosperm.

In some aspects, in vivo methods can utilize a truncated ZM-ODP2 polypeptide and a cyclin gene product such as ZM-CYCD2 to further stimulate stimulate partenogenic haploid induction and genetic chromosome doubling.

In another aspect, the disclosure provides methods of inducing somatic embryogenesis and genetic chromosome doubling in vitro using ZM-ODP2 (TR5) polynucleotide encoding a truncated ZM-ODP2 polypeptide. It will be understood that genetic chromosome doubling methods described herein are achieved without providing a chromosome doubling agent, for example a chemical chromosome doubling agent of Table 1, to a plant cell.

In some aspects, the disclosure provides methods to obtain doubled haploid plants from a single treated (e.g., transformed with a truncated ZM-ODP2 polypeptide) haploid embryo. In some aspects, the disclosure provides methods to obtain more than one regenerated plant per treated haploid embryo, providing a utility to obtain clonal siblings.

In some aspects, a method of producing a doubled haploid plant comprises: (a) inducing somatic embryogenesis in a haploid embryo; (b) transforming the haploid embryo with a polynucleotide sequence encoding a truncated ZM-ODP2 polypeptide; (c) obtaining a somatic embryo or somatic embryogenic tissue expressing the polynucleotide sequence encoding the truncated ZM-ODP2 polypeptide; (d) culturing the somatic embryo or somatic embryogenic tissue to obtain a plantlet, wherein a haploid set of chromosomes is diploidized; and (e) regenerating a doubled haploid plant from the plantlet or a mature seed thereof. In some aspects, to induce somatic embryogenesis, the haploid embryo can be transformed with a secondary morphogenic developmental expression gene (i.e., in addition to ZM-ODP2 (TR5)), such as, WUS/WOX genes and other BBM (ODP2) genes and variants.

In some aspects, the in vitro methods disclosed herein can provide genotypic data from a regenerated plant, which in turn, can be used to predict phenotypic performance of the plant, thereby resulting in a method to select, or enrich, a population comprising desired genotypes and with reduced levels of undesirable genotypes.

In some aspects, the combined activity of WUS and a truncated ZM-ODP2 polypeptide can further improve induction of somatic embryogenesis and/or in vitro chromosome doubling.

In some aspects, the combined activity of WUS and a truncated ZM-ODP2 polypeptide can further improve regeneration of fertile doubled haploid plants capable of producing intact, mature seed. In yet another aspect, the disclosure provides methods of producing genome-edited doubled haploid plants in vivo and in vitro.

Site specific modifications that can be introduced with the disclosed methods include those produced using any method for introducing site specific modification, including, but not limited to, through the use of gene repair oligonucleotides (e.g., US Publication 2013/0019349), or through the use of double-stranded break technologies such as TALENs, meganucleases, zinc finger nucleases, CRISPR-Cas, and the like. For example, the disclosed methods can be used to introduce a CRISPR-Cas system into a plant cell or plant, for the purpose of genome modification of a target sequence in the genome of a plant or plant cell, for selecting plants, for deleting a base or a sequence, for gene editing, and for inserting a polynucleotide of interest into the genome of a plant or plant cell. Thus, the disclosed methods can be used together with a CRISPR-Cas system to provide for an effective system for modifying or altering target sites and nucleotides of interest within the genome of a plant, plant cell or seed. In some aspects, the Cas endonuclease gene is a plant optimized Cas9 endonuclease, wherein the plant optimized Cas9 endonuclease is capable of binding to and creating a double strand break in a genomic target sequence of the plant genome.

In some aspects, a method of producing a genome-edited double haploid plant comprises: (a) providing a diploid embryo of a plant with a polynucleotide sequence encoding a truncated ZM-ODP2 polypeptide and a polynucleotide sequence encoding at least one genome-editing component; (b) regenerating a T₀ plant from the diploid embryo, wherein the T₀ plant expresses the polynucleotide sequence encoding the truncated ZM-ODP2 polypeptide and the polynucleotide sequence encoding the genome-editing component; (c) obtaining a donor ear from the T₀ plant; (d) pollinating the donor ear with pollen from a pollen donor (e.g., non-haploid inducer or haploid inducer); (e) selecting a haploid embryo that expresses the polynucleotide sequence encoding the truncated ZM-ODP2 polypeptide and the polynucleotide sequence encoding the one genome-editing component, and lacks the genome of the haploid inducer plant, wherein the truncated ZM-ODP2 polypeptide promotes chromosome doubling of the haploid embryo to produce a doubled haploid embryo; and (f) regenerating a doubled haploid plant from the doubled haploid embryo or a mature seed thereof.

In some aspects, female plants providing the donor ears can be grown to maturity and mature seed harvested and scored for the presence/absence of the paternal color marker. For this method, it is anticipated a seed expressing the paternal reporter gene is discarded; a seed without said paternal activity is retained.

In some aspects, a seed without paternal activity can be sampled in a non-destructive manner, for the purpose of DNA isolation. The isolated DNA can be used for a diagnostic PCR-based assay to detect presence/absence of any transgene, including determining the copy number of any transgenic construct, and for genotyping using PCR-based genetic marker assay methods known in the art. The genotypic data can be used to determine allelic states inherited at genome-wide marker loci useful for breeding selection methods known in the art, such as an estimate to calculate genomic estimated breeding values (GEBVs) for individuals without first having phenotypic data. Such genomic estimated breeding values (GEBVs) provide a method for identifying doubled haploid progeny lacking desirable genotypes that can be discarded and doubled haploid progeny possessing desirable genotypes to retain, thereby providing a method to enrich the structure of a doubled haploid population to be optimized for breeding purposes. Thus, the disclosure provides methods that produce a doubled haploid population that is both enriched for desirable genotypes, wherein a proportion of these desirable genotypes can also have a targeted genome modification.

In some aspects, the method of producing a genome-edited double haploid plant can comprise using isolated DNA for molecular characterization of genome modification, such as characterization of site directed nuclease activity at the genomic target site(s), and at off-target sites. For example, a double-stranded break (DSB) at any one gRNA target site without the addition of foreign DNA can cause a mutation or small deletion as an example of a first outcome for a site directed nuclease (SDN), hereinafter an “SDN-1 method”. It is also possible double strand breaks by a Cas nuclease at two gRNA target sites can occur causing subsequent DNA repair, for example non-homologous end joining. This exemplifies a second SDN outcome, that is a two-gRNA “drop-out”, hereinafter an “SDN-2 method”.

As the disclosed method provides simultaneous genome modification, parthenogenic haploid induction, and in vivo chromosome doubling activities to an unfertilized egg cell, in some aspects, the method further provides a subset of diploidized plants that produce fertile, genome modified, doubled haploid plants.

Further, in some aspects, the method does not require first creating a stable transgenic haploid inducer strain possessing the genome modification expression cassette that can optionally also require providing a gRNA expression cassette. Hence, the current disclosure enables a method for obtaining a genome-modified doubled haploid population with simplified logistics, in less time, and with less labor.

In some aspects, as the in vivo methods described herein can integrate genome-modification directly into the breeding process, the in vivo chromosome doubling method can improve the breeding process by increasing the proportion of selectable, genome-modified individuals per population.

In some aspects, the methods of producing genome-edited double haploid plant can utilize the ZM-ODP2-(266-668):At-CBF1a fusion protein. In some aspects, providing a ZM-ODP2-(266-668):At-CBF1a fusion protein to an unfertilized egg cell improves parthenogenic haploid induction and genetic chromosome doubling responses. In some aspects, the increased parthenogenic haploid induction and genetic chromosome doubling responses increase the resulting proportion of genome-modified, doubled haploid embryos.

In some aspects, a method of producing a genome-edited double haploid plant comprises: (a) inducing somatic embryogenesis in a haploid embryo; (b) transforming the haploid embryo with a polynucleotide sequence encoding a truncated ZM-ODP2 polypeptide; and a polynucleotide sequence encoding at least one genome-editing component; (c) obtaining a somatic embryo or somatic embryogenic tissue expressing the polynucleotide sequence encoding the truncated ZM-ODP2 polypeptide and the polynucleotide sequence encoding the genome-editing component; (d) culturing the somatic embryo or somatic embryogenic tissue to obtain a plantlet, wherein a haploid set of chromosomes is diploidized; and (e) regenerating a doubled haploid plant from the plantlet or a mature seed thereof. In some aspects, to induce somatic embryogenesis, the haploid embryo can be transformed with a secondary morphogenic developmental expression gene (i.e., in addition to ZM-ODP2 (TR5)), such as, WUS/WOX genes and other BBM (ODP2) genes and variants.

In some aspects, the combined activity of WUS and a ZM-ODP2-(266-669) polypeptide improves the level of somatic embryogenesis activity, in vitro chromosome doubling, and/or the frequency of regenerating genome-modified doubled haploid plants capable of producing mature, intact seed without the need for providing an artificial, chemical chromosome doubling treatment.

It will be understood that genetic chromosome doubling in the genome-editing methods described herein are achieved without providing a chromosome doubling agent, for example a chemical chromosome doubling agent of Table 1, to a plant cell.

In some aspects, the genome-editing component is a site-directed nuclease such as meganucleases (MNs), zinc-finger nucleases (ZFN), transcription-activator like effector nucleases (TALENs), Cas9 nuclease, Cas alpha nuclease, Cpf1 nuclease, dCas9-FokI, dCpf1-FokI, chimeric Cas9-cytidine deaminase, chimeric Cas9 adenine deaminase, chimeric FEN1-Fok1, Mega-TALs, a nickase Cas9 (nCas9), chimeric dCas9 non-FokI nuclease, and dCpf1-non-FokI nuclease.

In some aspects, more than one genome-editing component can be provided. For example, a first polynucleotide sequence encoding a Cas9 endonuclease and a second polynucleotide sequence encoding a guide RNA. Alternatively, a first polynucleotide sequence encoding a Cas alpha endonuclease and a second polypeptide sequence encoding a guide RNA.

The Cas endonuclease is guided by the guide nucleotide to recognize and optionally introduce a double strand break at a specific target site into the genome of a cell. The CRISPR-Cas system provides for an effective system for modifying target sites within the genome of a plant, plant cell or seed. Further provided are methods employing a guide polynucleotide/Cas endonuclease system to provide an effective system for modifying target sites within the genome of a cell and for editing a nucleotide sequence in the genome of a cell. Once a genomic target site is identified, a variety of methods can be employed to further modify the target sites such that they contain a variety of polynucleotides of interest. The disclosed methods can be used to introduce a CRISPR-Cas system for editing a nucleotide sequence in the genome of a cell. The nucleotide sequence to be edited (the nucleotide sequence of interest) can be located within or outside a target site that is recognized by a Cas endonuclease.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

Cas gene includes a gene that is generally coupled, associated or close to or in the vicinity of flanking CRISPR loci. The terms “Cas gene” and “CRISPR-associated (Cas) gene” are used interchangeably herein.

In another aspect, the Cas endonuclease gene is operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region.

As related to the Cas endonuclease, the terms “functional fragment,” “fragment that is functionally equivalent,” and “functionally equivalent fragment” are used interchangeably herein. These terms refer to a portion or subsequence of the Cas endonuclease sequence in which the ability to create a double-strand break is retained.

As related to the Cas endonuclease, the terms “functional variant,” “variant that is functionally equivalent” and “functionally equivalent variant” are used interchangeably herein. These terms refer to a variant of the Cas endonuclease in which the ability to create a double-strand break is retained. Fragments and variants can be obtained via methods such as site-directed mutagenesis and synthetic construction.

In an aspect, the Cas endonuclease gene is a plant codon optimized Streptococcus pyogenes Cas9 gene that can recognize any genomic sequence of the form N(12-30)NGG which can in principle be targeted.

Endonucleases are enzymes that cleave the phosphodiester bond within a polynucleotide chain and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (Patent application PCT/US 12/30061 filed on Mar. 22, 2012). Meganucleases have been classified into four families based on conserved sequence motifs. These motifs participate in the coordination of metal ions and hydrolysis of phosphodiester bonds. Meganucleases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families. TAL effector nucleases are a new class of sequence-specific nucleases that can be used to make double-strand breaks at specific target sequences in the genome of a plant or other organism. (Miller, et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a nonspecific endonuclease domain, for example nuclease domain from a Type Ms endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

A “Dead-CAS9” (dCAS9) as used herein, is used to supply a transcriptional repressor domain. The dCAS9 has been mutated so that can no longer cut DNA. The dCAS0 can still bind when guided to a sequence by the gRNA and can also be fused to repressor elements. The dCAS9 fused to the repressor element, as described herein, is abbreviated to dCAS9-REP, where the repressor element (REP) can be any of the known repressor motifs that have been characterized in plants. An expressed guide RNA (gRNA) binds to the dCAS9-REP protein and targets the binding of the dCAS9-REP fusion protein to a specific predetermined nucleotide sequence within a promoter (a promoter within the T-DNA). For example, if this is expressed beyond—the border using a ZM-UBI PRO::dCAS9˜REP::PINII TERM cassette along with a U6-POL PRO::gRNA::U6 TERM cassette and the gRNA is designed to guide the dCAS9-REP protein to bind the SB-UBI promoter in the expression cassette SB-UBI PRO::moPAT::PINII TERM within the T-DNA, any event that has integrated the beyond-the-border sequence would be bialaphos sensitive. Transgenic events that integrate only the T-DNA would express moPAT and be bialaphos resistant. The advantage of using a dCAS9 protein fused to a repressor (as opposed to a TETR or ESR) is the ability to target these repressors to any promoter within the T-DNA. TETR and ESR are restricted to cognate operator binding sequences. Alternatively, a synthetic Zinc-Finger Nuclease fused to a repressor domain can be used in place of the gRNA and dCAS9-REP (Urritia et al., 2003, Genome Biol. 4:231) as described above.

The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target. As used herein, the term “guide nucleotide” relates to a synthetic fusion of two RNA molecules, a crRNA (CRISPR RNA) comprising a variable targeting domain, and a tracrRNA. In an aspect, the guide nucleotide comprises a variable targeting domain of 12 to 30 nucleotide sequences and a RNA fragment that can interact with a Cas endonuclease.

As used herein, the term “guide polynucleotide” relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide nucleotide”.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In an aspect, the guide nucleotide and Cas endonuclease are capable of forming a complex that enables the Cas endonuclease to introduce a double strand break at a DNA target site.

In an aspect of the present disclosure the variable target domain is 12, 13, 14, 15, 16, 17, 18, 19, 20, 21, 22, 23, 24, 25, 26, 27, 28, 29 or 30 nucleotides in length.

In an aspect of the present disclosure, the guide nucleotide comprises a cRNA (or cRNA fragment) and a tracrRNA (or tracrRNA fragment) of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. The guide nucleotide can be introduced into a plant or plant cell directly using any method known in the art such as, but not limited to, particle bombardment or topical applications.

In an aspect, the guide nucleotide can be introduced indirectly by introducing a recombinant DNA molecule comprising the corresponding guide DNA sequence operably linked to a plant specific promoter that is capable of transcribing the guide nucleotide in the plant cell. The term “corresponding guide DNA” includes a DNA molecule that is identical to the RNA molecule but has a “T” substituted for each “U” of the RNA molecule.

In an aspect, the guide nucleotide is introduced via particle bombardment or using the disclosed methods for Agrobacterium transformation of a recombinant DNA construct comprising the corresponding guide DNA operably linked to a plant U6 polymerase III promoter.

In an aspect, the RNA that guides the RNA Cas9 endonuclease complex, is a duplexed RNA comprising a duplex crRNA-tracrRNA. One advantage of using a guide nucleotide versus a duplexed crRNA-tracrRNA is that only one expression cassette needs to be made to express the fused guide nucleotide.

The terms “target site,” “target sequence,” “target DNA,” “target locus,” “genomic target site,” “genomic target sequence,” and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature.

As used herein, terms “endogenous target sequence” and “native target sequence” are used interchangeably herein to refer to a target sequence that is endogenous or native to the genome of a plant and is at the endogenous or native position of that target sequence in the genome of the plant.

An “artificial target site” or “artificial target sequence” are used interchangeably herein and refer to a target sequence that has been introduced into the genome of a plant. Such an artificial target sequence can be identical in sequence to an endogenous or native target sequence in the genome of a plant but be located in a different position (i.e., a non-endogenous or non-native position) in the genome of a plant.

An “altered target site,” “altered target sequence” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

In an aspect, the disclosed methods can be used to introduce into plants polynucleotides useful for gene suppression of a target gene in a plant. Reduction of the activity of specific genes (also known as gene silencing, or gene suppression) is desirable for several aspects of genetic engineering in plants. Many techniques for gene silencing are well known to one of skill in the art, including but not limited to antisense technology.

In an aspect, the disclosed methods can be used to introduce into plants polynucleotides useful for the targeted integration of nucleotide sequences into a plant. For example, the disclosed methods can be used to introduce T-DNA expression cassettes comprising nucleotide sequences of interest flanked by non-identical recombination sites are used to transform a plant comprising a target site. In an aspect, the target site contains at least a set of non-identical recombination sites corresponding to those on the T-DNA expression cassette. The exchange of the nucleotide sequences flanked by the recombination sites is affected by a recombinase. Thus, the disclosed methods can be used for the introduction of T-DNA expression cassettes for targeted integration of nucleotide sequences, wherein the T-DNA expression cassettes which are flanked by non-identical recombination sites recognized by a recombinase that recognizes and implements recombination at the nonidentical recombination sites. Accordingly, the disclosed methods and composition can be used to improve efficiency and speed of development of plants containing non-identical recombination sites.

Thus, the disclosed methods can further comprise methods for the directional, targeted integration of exogenous nucleotides into a transformed plant. In an aspect, the disclosed methods use novel recombination sites in a gene targeting system which facilitates directional targeting of desired genes and nucleotide sequences into corresponding recombination sites previously introduced into the target plant genome.

In an aspect, a nucleotide sequence flanked by two non-identical recombination sites is introduced into one or more cells of an explant derived from the target organism's genome establishing a target site for insertion of nucleotide sequences of interest. Once a stable plant or cultured tissue is established a second construct, or nucleotide sequence of interest, flanked by corresponding recombination sites as those flanking the target site, is introduced into the stably transformed plant or tissues in the presence of a recombinase protein. This process results in exchange of the nucleotide sequences between the non-identical recombination sites of the target site and the T-DNA expression cassette.

It is recognized that the transformed plant prepared in this manner may comprise multiple target sites; i.e., sets of non-identical recombination sites. In this manner, multiple manipulations of the target site in the transformed plant are available. By target site in the transformed plant is intended a DNA sequence that has been inserted into the transformed plant's genome and comprises non-identical recombination sites.

Examples of recombination sites for use in the disclosed method are known. The two-micron plasmid found in most naturally occurring strains of Saccharomyces cerevisiae, encodes a site-specific recombinase that promotes an inversion of the DNA between two inverted repeats. This inversion plays a central role in plasmid copy-number amplification.

The protein, designated FLP protein, catalyzes site-specific recombination events. The minimal recombination site (FRT) has been defined and contains two inverted 13-base pair (bp) repeats surrounding an asymmetric 8-bp spacer. The FLP protein cleaves the site at the junctions of the repeats and the spacer and is covalently linked to the DNA via a 3′phosphate. Site specific recombinases like FLP cleave and religate DNA at specific target sequences, resulting in a precisely defined recombination between two identical sites. To function, the system needs the recombination sites and the recombinase. No auxiliary factors are needed. Thus, the entire system can be inserted into and function in plant cells. The yeast FLP\FRT site specific recombination system has been shown to function in plants. To date, the system has been utilized for excision of unwanted DNA. See, Lyznik et at. (1993) Nucleic Acid Res. 21: 969-975. In contrast, the present disclosure utilizes non-identical FRTs for the exchange, targeting, arrangement, insertion and control of expression of nucleotide sequences in the plant genome.

In an aspect, a transformed organism of interest, such as an explant from a plant, containing a target site integrated into its genome is needed. The target site is characterized by being flanked by non-identical recombination sites. A targeting cassette is additionally required containing a nucleotide sequence flanked by corresponding non-identical recombination sites as those sites contained in the target site of the transformed organism. A recombinase which recognizes the non-identical recombination sites and catalyzes site-specific recombination is required.

It is recognized that the recombinase can be provided by any means known in the art. That is, it can be provided in the organism or plant cell by transforming the organism with an expression cassette capable of expressing the recombinase in the organism, by transient expression, or by providing messenger RNA (mRNA) for the recombinase or the recombinase protein.

By “non-identical recombination sites” it is intended that the flanking recombination sites are not identical in sequence and will not recombine or recombination between the sites will be minimal. That is, one flanking recombination site may be a FRT site where the second recombination site may be a mutated FRT site. The non-identical recombination sites used in the methods of the present disclosure prevent or greatly suppress recombination between the two flanking recombination sites and excision of the nucleotide sequence contained therein. Accordingly, it is recognized that any suitable non-identical recombination sites may be utilized in the present disclosure, including FRT and mutant FRT sites, FRT and lox sites, lox and mutant lox sites, as well as other recombination sites known in the art.

By suitable non-identical recombination site implies that in the presence of active recombinase, excision of sequences between two non-identical recombination sites occurs, if at all, with an efficiency considerably lower than the recombinationally-mediated exchange targeting arrangement of nucleotide sequences into the plant genome. Thus, suitable non-identical sites for use in the present disclosure include those sites where the efficiency of recombination between the sites is low; for example, where the efficiency is less than about 30 to about 50%, preferably less than about 10 to about 30%, more preferably less than about 5 to about 10%.

As noted above, the recombination sites in the targeting cassette correspond to those in the target site of the transformed plant. That is, if the target site of the transformed plant contains flanking non-identical recombination sites of FRT and a mutant FRT, the targeting cassette will contain the same FRT and mutant FRT non-identical recombination sites.

It is furthermore recognized that the recombinase, which is used in the disclosed methods, will depend upon the recombination sites in the target site of the transformed plant and the targeting cassette. That is, if FRT sites are utilized, the FLP recombinase will be needed. In the same manner, where lox sites are utilized, the Cre recombinase is required. If the non-identical recombination sites comprise both a FRT and a lox site, both the FLP and Cre recombinase will be required in the plant cell.

The FLP recombinase is a protein which catalyzes a site-specific reaction that is involved in amplifying the copy number of the two-micron plasmid of S. cerevisiae during DNA replication. FLP protein has been cloned and expressed. See, for example, Cox (1993) Proc. Natl. Acad. Sci. U.S.A 80: 4223-4227. The FLP recombinase for use in the present disclosure may be that derived from the genus Saccharomyces. It may be preferable to synthesize the recombinase using plant preferred codons for optimum expression in a plant of interest. See, for example, U.S. application Ser. No. 08/972,258 filed Nov. 18, 1997, entitled “Novel Nucleic Acid Sequence Encoding FLP Recombinase,” herein incorporated by reference.

The bacteriophage recombinase Cre catalyzes site-specific recombination between two lox sites. The Cre recombinase is known in the art. See, for example, Guo et al. (1997) Nature 389: 40-46; Abremski et al. (1984) J. Biol. Chem. 259: 1509-1514; Chen et al. (1996) Somat. Cell Mol. Genet. 22: 477-488; and Shaikh et al. (1977) J. Biol. Chem. 272: 5695-5702. All of which are herein incorporated by reference. Such Cre sequence may also be synthesized using plant preferred codons.

Where appropriate, the nucleotide sequences to be inserted in the plant genome may be optimized for increased expression in the transformed plant. Where mammalian, yeast, or bacterial genes are used in the present disclosure, they can be synthesized using plant preferred codons for improved expression. It is recognized that for expression in monocots, dicot genes can also be synthesized using monocot preferred codons. Methods are available in the art for synthesizing plant preferred genes. See, for example, U.S. Pat. Nos. 5,380,831, 5,436,391, and Murray et al. (1989) Nucleic Acids Res. 17: 477-498, herein incorporated by reference. The plant preferred codons may be determined from the codons utilized more frequently in the proteins expressed in the plant of interest. It is recognized that monocot or dicot preferred sequences may be constructed as well as plant preferred sequences for particular plant species. See, for example, EPA 0359472; EPA 0385962; WO 91/16432; Perlak et al. (1991) Proc. Natl. Acad. Sci. USA, 88: 3324-3328; and Murray et al. (1989) Nucleic Acids Research, 17: 477-498. U.S. Pat. Nos. 5,380,831; 5,436,391; and the like, herein incorporated by reference. It is further recognized that all or any part of the gene sequence may be optimized or synthetic. That is, fully optimized or partially optimized sequences may also be used.

Additional sequence modifications are known to enhance gene expression in a cellular host and can be used in the present disclosure. These include elimination of sequences encoding spurious polyadenylation signals, exon-intron splice site signals, transposon-like repeats, and other such well-characterized sequences, which may be deleterious to gene expression. The G-C content of the sequence may be adjusted to levels average for a given cellular host, as calculated by reference to known genes expressed in the host cell. When possible, the sequence is modified to avoid predicted hairpin secondary RNA structures.

The present disclosure also encompasses FLP recombination target sites (FRT). The FRT has been identified as a minimal sequence comprising two 13 base pair repeats, separated by an eight (8) base spacer. The nucleotides in the spacer region can be replaced with a combination of nucleotides, so long as the two 13-base repeats are separated by eight nucleotides. It appears that the actual nucleotide sequence of the spacer is not critical; however, for the practice of the present disclosure, some substitutions of nucleotides in the space region may work better than others. The eight-base pair spacer is involved in DNA-DNA pairing during strand exchange. The asymmetry of the region determines the direction of site alignment in the recombination event, which will subsequently lead to either inversion or excision. As indicated above, most of the spacer can be mutated without a loss of function. See, for example, Schlake and Bode (1994) Biochemistry 33: 12746-12751, herein incorporated by reference.

FRT mutant sites can be used in the practice of the disclosed methods. Such mutant sites may be constructed by PCR-based mutagenesis. Although mutant FRT sites are known (see SEQ ID Nos 2, 3, 4 and 5 of WO1999/025821), it is recognized that other mutant FRT sites may be used in the practice of the present disclosure. The present disclosure is not restricted to the use of a particular FRT or recombination site, but rather that non-identical recombination sites or FRT sites can be utilized for targeted insertion and expression of nucleotide sequences in a plant genome. Thus, other mutant FRT sites can be constructed and utilized based upon the present disclosure.

As discussed above, bringing genomic DNA containing a target site with non-identical recombination sites together with a vector containing a T-DNA expression cassette with corresponding non-identical recombination sites, in the presence of the recombinase, results in recombination. The nucleotide sequence of the T-DNA expression cassette located between the flanking recombination sites is exchanged with the nucleotide sequence of the target site located between the flanking recombination sites. In this manner, nucleotide sequences of interest may be precisely incorporated into the genome of the host.

It is recognized that many variations of the present disclosure can be practiced. For example, target sites can be constructed having multiple non-identical recombination sites. Thus, multiple genes or nucleotide sequences can be stacked or ordered at precise locations in the plant genome. Likewise, once a target site has been established within the genome, additional recombination sites may be introduced by incorporating such sites within the nucleotide sequence of the T-DNA expression cassette and the transfer of the sites to the target sequence. Thus, once a target site has been established, it is possible to subsequently add sites, or alter sites through recombination.

Another variation includes providing a promoter or transcription initiation region operably linked with the target site in an organism. Preferably, the promoter will be 5′ to the first recombination site. By transforming the organism with a T-DNA expression cassette comprising a coding region, expression of the coding region will occur upon integration of the T-DNA expression cassette into the target site. This aspect provides for a method to select transformed cells, particularly plant cells, by providing a selectable marker sequence as the coding sequence.

Other advantages of the present system include the ability to reduce the complexity of integration of transgenes or transferred DNA in an organism by utilizing T-DNA expression cassettes as discussed above and selecting organisms with simple integration patterns. In the same manner, preferred sites within the genome can be identified by comparing several transformation events. A preferred site within the genome includes one that does not disrupt expression of essential sequences and provides for adequate expression of the transgene sequence.

In yet a further aspect, the disclosure provides methods of seed sorting in doubled haploid plants, wherein the method comprises: (a) providing a diploid embryo of a plant with a polynucleotide sequence encoding a truncated ZM-ODP2 polypeptide; (b) regenerating a T₀ plant from the diploid embryo, wherein the T₀ plant expresses the polynucleotide sequence encoding the truncated ZM-ODP2 polypeptide; (c) obtaining a donor ear from the T₀ plant; (d) pollinating the donor ear with pollen from a pollen donor plant; (e) selecting a haploid embryo expressing the polynucleotide sequence encoding the truncated ZM-ODP2 polypeptide and lacking the genome of the haploid inducer plant, wherein the truncated ZM-ODP2 polypeptide promotes chromosome doubling of the haploid embryo to produce a doubled haploid embryo; (f) regenerating a doubled haploid plant from the doubled haploid embryo or a mature seed thereof; and (g) selecting a maternally-derived doubled haploid seed based on the absence of a paternal marker gene caused by parthenogenic haploid induction of a maternal egg cell.

In some aspects, selecting the maternally-derived doubled haploid seed comprising using a manual method or an automated method.

In some aspects, the automated method uses machine vision and/or machine learning methods.

Further, the methods described herein can use automation for producing clonal plants. For example, clonal propagation activities are conceived as being performed using an apparatus for preparing a plant tissue, to hold a tissue, for applying an Agrobacterium to a plant tissue, for transferring plant tissue, for culturing plant tissue, and or for subjecting the tissue to a force to divide the tissue into separate segments. It is expected such steps may use more than one apparatus. In some aspects, automation steps can also comprise integrating analytical capabilities including but not limited to use of different sensors and image capture systems. For example, an apparatus may be used for acquiring an image, such as visual, hyperspectral, thermal, or fluorescence imaging. In another example, an apparatus may be used for measuring, capturing, and analyzing qualitative and quantitative traits, such as biomass, shape, thickness, volume, growth rate, and or morphological characteristics. The apparatus may also be used for measuring and analyzing of other parameters, such as light intensity, light duration, water and nutrient uptake, evaporation and transpiration components, and or quantitative measurement of the complete set of ions or changes in ion production under varied external stimuli.

In some aspects, such methods can include performing feature extraction for classification purposes, wherein such classification can be used for predictive model generation. Such predictive modelling is a computer implemented model encompassing a variety of statistical techniques from data mining, automated feature extraction, and machine learning.

In some aspects, data analysis can comprise linking genotypic data with captured phenotypic data, including methods measuring biomolecules from a tissue before, during, or after such automation methods are performed.

In some aspects, these automated methods can be linked with genotypic data, for example to enable predicting phenotypic performance using a biological model based on genomic data of a characterized plant, tissue, or plant cell treated using methods of the present disclosure.

In some aspects, the methods of the present disclosure can improve the capability for producing clonal plants, including but not limited to aspects for improving the regeneration frequency, improved transplanting success, and ultimately improvements for the reproductive success of the clonal plants produced using such automated treatment, handling, and phenotyping methods.

In some aspects, the results of linking genotypic data with the acquired phenotypic data can enable improving predicting phenotypic performance using a biological model based on genomic data of a characterized plant, tissue, or plant cell treated.

In some aspects, the methods of the present disclosure can improve plant breeding outcomes, for example when such automated treatment, handling, and phenotyping methods that co-integrate the above data analysis and interpretation with the method of selecting clonal doubled haploid plants based on a genomic estimated breeding value. It is expected this method can provide a novel capability to improve the productivity of a non-random, structured breeding population. It is expected this result can more cost-effectively provide a population with the number of individuals required to have some specified quantity of interest. Thus, in comparison to having the same probability in the idealized population, such as the effective population size required when provided a random population, the methods here are expected to improve the relative rate of genetic gain while using relatively fewer resources.

### EXAMPLES

The aspects of the disclosure are further defined in the following Examples, in which parts and percentages are by weight and degrees are Celsius, unless otherwise stated. These Examples, while indicating aspects of the disclosure, are given by way of illustration only. From the above discussion and these Examples, one skilled in the art can ascertain the essential characteristics of the aspects of the disclosure, and without departing from the spirit and scope thereof, can make various changes and modifications of them to adapt to various usages and conditions. Thus, various modifications in addition to those shown and described herein will be apparent to those skilled in the art from the foregoing description. Such modifications are also intended to fall within the scope of the appended claims.

### Example 1: Plasmids

Plasmids detailed in Table 3 were used in the Examples described herein.

TABLE 3
Plasmids

| SEQ ID | | |
|---|---|---|
| NO: | Plasmid | Plasmid Elements |
| 7 | RV027603 | RB + SB-ALS PRO::ZM-ALS (HRA) EXON1 + LOXP + ZM-AXIG1 |
| | | 1XOP-B PRO-V1::ZM-WUS2 (CDNA):IN2-1 TERM + AT-5-IV-2 |
| | | INS + ZM-PLTP PRO::ZM-PLTP 5 UTR:ZM-ODP2 (ALT1):OS-T28 |
| | | TERM:PINII TERM:CZ19B1 TERM + ZM-GLB1 PRO::MO-CRE |
| | | EXON1:ST-LS1 INTRON2-V2:MO-CRE EXON2:PINII TERM-V3 + |
| | | SB-UBI PRO::SB-UBI INTRON1:ZS-GREEN1:OS-UBI TERM + |
| | | LOXP + ZM-ALS (HRA) EXON2:SB-PEPC1 TERM (MOD1) + |
| | | PV-EGG CELL PRO (TR1):PV-PRO31696.1 5UTR:EGG MIN |
| | | PRO::ZM-ODP2 (TR5):PINII TERM + LB |
| 16 | RV048759 | RB + SB-ALS PRO::ZM-ALS (HRA) EXON1 + LOXP + ZM-AXIG1 |
| | | 1XOP-B PRO-V1::ZM-WUS2 (CDNA):IN2-1 TERM + AT-5-IV-2 |
| | | INS + ZM-PLTP PRO::ZM-PLTP 5 UTR:ZM-ODP2 (ALT1):OS-T28 |
| | | TERM:PINII TERM:CZ19B1 TERM + ZM-GLB1 PRO::MO-CRE |
| | | EXON1:ST-LS1 INTRON2-V2:MO-CRE EXON2:PINII TERM-V3 + |
| | | SB-UBI PRO::SB-UBI INTRON1:ZS-GREEN1:OS-UBI TERM + |
| | | LOXP + ZM-ALS (HRA) EXON2:SB-PEPC1 TERM (MOD1):AT- |
| | | RPG ELEMENT-V3 + PV-EGG CELL PRO (TR1):PV-PRO31696.1 |
| | | 5UTR:EGG MIN PRO::ZM-ODP2 (TR5)-V1:PROTEIN LINKER 1 |
| | | (MOD1):AT-CBF1A (MO1) (TR1) (MOD1):PINII TERM + LB |
| 19 | RV020636 | RB + LOXP + FMV ENHANCER:PCSV ENH:MMV ENH:ZM- |
| | | PLTP1 PRO (3X ZM-AS2 EME)::ZM-WUS2 (ALT1):IN2-1 TERM: + |
| | | NOS PRO::CRC (ALT6):SB-GKAF TERM (MOD1) + LOXP + LB |
| 20 | RV047438 | RB + LOXP + ZM-GLB1 PRO::MO-CRE EXON1:ST-LS1 |
| | | INTRON2-V2:MO-CRE EXON2:PINII TERM + LOXP + UBI1ZM |
| | | PRO::UBI1ZM 5UTR (PHI):UBI1ZM INTRON1 (PHI):NPTII:PINII |
| | | TERM + LB |
| 21 | RV047990 | RB + LOXP + UBI1ZM PRO::UBI1ZM 5UTR (PHI):UBI1ZM |
| | | INTRON1 (PHI):ZM-ODP2 (TR5):PINII TERM + ZM-GLB1 |
| | | PRO::MO-CRE EXON1:ST-LS1 INTRON2-V2:MO-CRE |
| | | EXON2:PINII TERM + LOXP + UBI1ZM PRO::UBI1ZM 5UTR |
| | | (PHI):UBI1ZM INTRON1 (PHI):NPTII:PINII TERM + LB |
| 22 | RV047991 | RB + LOXP + ZM-PLTP PRO::ZM-PLTP 5 UTR:ZM-ODP2 |
| | | (TR5):OS-T28 TERM + ZM-GLB1 PRO::MO-CRE EXON1:ST-LS1 |
| | | INTRON2-V2:MO-CRE EXON2:PINII TERM + LOXP + UBI1ZM |
| | | PRO::UBI1ZM 5UTR (PHI):UBI1ZM INTRON1 (PHI):NPTII:PINII |
| | | TERM + LB |
| 23 | RV048128 | RB + LOXP + UBI1ZM PRO::UBI1ZM 5UTR (PHI):UBI1ZM |
| | | INTRON1 (PHI):SV40 NLS:CAS9 EXON1 (SP) (MO):ST-LS1 |
| | | INTRON2:CAS9 EXON2 (SP) (MO):VIRD2 NLS:PINII TERM + |
| | | ZM-U6 POLIII CHR8 PRO::ZM-NAC7-CR5 GUIDE RNA:ZM-U6 |
| | | POLIII CHR8 TERM + ZM-U6 POLIII CHR8 PRO::ZM-NAC7-CR5 |
| | | GUIDE RNA:ZM-U6 POLIII CHR8 TERM + ZM-GLB1 PRO::MO- |
| | | CRE EXON1:ST-LS1 INTRON2-V2:MO-CRE EXON2:PINII TERM + |
| | | UBI1ZM PRO::UBI1ZM 5UTR (PHI):UBI1ZM INTRON1 |
| | | (PHI):ZM-ODP2 (TR5):PINII TERM + LOXP + UBI1ZM |
| | | PRO:UBI1ZM 5UTR (PHI):UBI1ZM INTRON1 (PHI):NPTII:PINII |
| | | TERM + LB |
| 24 | RV048055 | RB + LOXP + UBI1ZM PRO::UBI1ZM 5UTR (PHI):UBI1ZM |
| | | INTRON1 (PHI):SV40 NLS:CAS9 EXON1 (SP) (MO):ST-LS1 |
| | | INTRON2:CAS9 EXON2 (SP) (MO):VIRD2 NLS:PINII TERM + |
| | | ZM-U6 POLIII CHR8 PRO::ZM-NAC7-CR5 GUIDE RNA:ZM-U6 |
| | | POLIII CHR8 TERM + ZM-U6 POLIII CHR8 PRO::ZM-NAC7-CR5 |
| | | GUIDE RNA:ZM-U6 POLIII CHR8 TERM + ZM-GLB1 PRO::MO- |
| | | CRE EXON1:ST-LS1 INTRON2-V2:MO-CRE EXON2:PINII TERM + |
| | | ZM-PLTP PRO::ZM-PLTP 5 UTR:ZM-ODP2 (TR5):PINII TERM + |
| | | LOXP + UBI1ZM PRO::UBI1ZM 5UTR (PHI):UBI1ZM INTRON1 |
| | | (PHI):NPTII:PINII TERM + LB |
| 47 | RV034410 | RB + LOXP + PV-EGG CELL PRO (TR1)::PV-PRO31696.1 |
| | | 5UTR:EGG MIN PRO:ZM-ODP2 (TR5):PINII TERM + ZM- |
| | | EXP31554.1 PRO-V1::SV40 NLS:CAS9 EXON1 (SP) (MO):ST-LS1 |
| | | INTRON2:CAS9 EXON2 (SP) (MO):VIRD2 NLS:6FRAME |
| | | STOPS1:ZM-EGG TERM + ZM-U6 POLIII CHR8 PRO::ZM-NAC7- |
| | | CR5 GUIDE RNA:ZM-U6 POLIII CHR8 TERM + ZM-U6 POLIII |
| | | CHR8 PRO::ZM-NAC7-CR5 GUIDE RNA:ZM-U6 POLIII CHR8 |
| | | TERM + UBI1ZM PRO::UBI1ZM 5UTR (PHI):UBI1ZM INTRON1 |
| | | (PHI):DS-RED2 (TR1):PINII TERM + PV-EGG CELL PRO |
| | | (TR1):PV-PRO31696.1 5UTR:EGG MIN PRO::MO-CRE |
| | | EXON1:ST-LS1 INTRON2-V2:MO-CRE EXON2:PINII TERM + |
| | | UBI1ZM PRO::UBI1ZM 5UTR (PHI):UBI1ZM INTRON1 |
| | | (PHI):NPTII:PINII TERM + LOXP + LB |
| 48 | RV053058 | RB + LOXP + PV-EGG CELL PRO (TR1):PV-PRO31696.1 |
| | | 5UTR:EGG MIN PRO::ZM-ODP2 (TR5)-V1:PROTEIN LINKER 1 |
| | | (MOD1):AT-CBF1A (MO1) (TR1) (MOD1):PINII TERM + PV-EGG |
| | | CELL PRO (TR1):PV-PRO31696.1 5UTR:EGG MIN PRO::ZM- |
| | | CYCD2:PINII TERM + ZM-EXP31554.1 PRO-V1::SV40 NLS:CAS9 |
| | | EXON1 (SP) (MO):ST-LS1 INTRON2:CAS9 EXON2 (SP) |
| | | (MO):VIRD2 NLS:6FRAME STOPS1:ZM-EGG TERM + ZM-U6 |
| | | POLIII CHR8 PRO::ZM-NAC7-CR5 GUIDE RNA:ZM-U6 POLIII |
| | | CHR8 TERM + ZM-U6 POLIII CHR8 PRO::ZM-NAC7-CR5 GUIDE |
| | | RNA:ZM-U6 POLIII CHR8 TERM + UBI1ZM PRO::UBI1ZM 5UTR |
| | | (PHI):UBI1ZM INTRON1 (PHI):DS-RED2 (TR1):PINII TERM + |
| | | PV-EGG CELL PRO (TR1):PV-PRO31696.1 5UTR:EGG MIN |
| | | PRO::MO-CRE EXON1:ST-LS1 INTRON2-V2:MO-CRE |
| | | EXON2:PINII TERM + UBI1ZM PRO::UBI1ZM 5UTR |
| | | (PHI):UBI1ZM INTRON1 (PHI):NPTII:PINII TERM + + LOXP + |
| | | LB |
| 51 | RV048819 | RB + LOXP + PV-EGG CELL PRO (TR1):EGG MIN PRO::PV- |
| | | PRO31696.1 5UTR:ZM-ODP2 (TR5):PINII TERM + PV-EGG CELL |
| | | PRO (TR1):EGG MIN PRO::PV-PRO31696.1 5UTR:ZM- |
| | | CYCD2:PINII TERM + ZM-EXP31554.1 PRO-V1::SV40 NLS:CAS9 |
| | | EXON1 (SP) (MO):ST-LS1 INTRON2:CAS9 EXON2 (SP) |
| | | (MO):VIRD2 NLS:6FRAME STOPS1:ZM-EGG TERM + ZM-U6 |
| | | POLIII CHR8 PRO::ZM-NAC7-CR5:GUIDE RNA:ZM-U6 POLIII |
| | | CHR8 TERM + ZM-U6 POLIII CHR8 PRO::ZM-NAC7-CR5:GUIDE |
| | | RNA:ZM-U6 POLIII CHR8 TERM + UBI1ZM PRO::UBI1ZM 5UTR |
| | | (PHI):UBI1ZM INTRON1 (PHI):DS-RED2 (TR1):PINII TERM + |
| | | PV- EGG CELL PRO (TR1):EGG MIN PRO:PV-PRO31696.1 |
| | | 5UTR::MO-CRE EXON1:ST-LS1 INTRON2-V2:MO-CRE |
| | | EXON2:PINII TERM + UBI1ZM PRO::UBI1ZM 5UTR |
| | | (PHI):UBI1ZM INTRON1 (PHI):NPTII:PINII TERM + LOXP + LB |
| 52 | RV048931 | RB + LOXP + PV-EGG CELL PRO (TR1):EGG MIN PRO::PV- |
| | | PRO31696.1 5UTR:ZM-ODP2 (TR5)-V1:PROTEIN LINKER 1 |
| | | (MOD1):AT-CBF1A (MO1) (TR1) (MOD1):PINII TERM + PV-EGG |
| | | CELL PRO (TR1):EGG MIN PRO::PV-PRO31696.1 5UTR:ZM- |
| | | CYCD2:PINII TERM + ZM-EXP31554.1 PRO-V1::SV40 NLS:CAS9 |
| | | EXON1 (SP) (MO):ST-LS1 INTRON2:CAS9 EXON2 (SP) |
| | | (MO):VIRD2 NLS:6FRAME STOPS1:ZM-EGG TERM + ZM-U6 |
| | | POLIII CHR8 PRO::ZM-NAC7-CR5:GUIDE RNA:ZM-U6 POLIII |
| | | CHR8 TERM + ZM-U6 POLIII CHR8 PRO::ZM-NAC7-CR5:GUIDE |
| | | RNA:ZM-U6 POLIII CHR8 TERM + UBI1ZM PRO::UBI1ZM 5UTR |
| | | (PHI):UBI1ZM INTRON1 (PHI):DS-RED2 (TR1):PINII TERM + |
| | | PV- EGG CELL PRO (TR1):EGG MIN PRO:PV-PRO31696.1 |
| | | 5UTR::MO-CRE EXON1:ST-LS1 INTRON2-V2:MO-CRE |
| | | EXON2:PINII TERM + UBI1ZM PRO::UBI1ZM 5UTR |
| | | (PHI):UBI1ZM INTRON1 (PHI):NPTII:PINII TERM + LOXP + LB |

### Example 2: Agrobacterium-Mediated Transformation of Maize

### A. Preparation of an Agrobacterium Master Plate

Agrobacterium tumefaciens harboring a binary donor vector was streaked from a −80° C. frozen aliquot onto solid 12R medium and cultured in the dark at 28° C. for 2-3 days to make a master plate.

### B. Growing Agrobacterium on Solid Medium

A single colony or multiple colonies of Agrobacterium were picked from the master plate, streaked onto a second plate containing 810K medium, and incubated at 28° C. overnight in the dark. Agrobacterium infection medium (700A; 5 ml) and 100 mM 3′-5′-Dimethoxy-4′-hydroxyacetophenone (acetosyringone; 5 μL) were added to a 14-mL conical tube. About 3 full loops of Agrobacterium from the second plate were suspended in the tube, and the tube was then vortexed to make an even suspension. The suspension (1 ml) was transferred to a spectrophotometer tube and the optical density (550 nm) of the suspension was adjusted to a reading of about 0.35-1.0. The Agrobacterium concentration was approximately 0.5 to 2.0×10⁹ cfu/mL. The final Agrobacterium suspension was aliquoted into 2 mL microcentrifuge tubes, each containing about 1 mL of the suspension. The suspensions were used immediately.

### C. Growing Agrobacterium on Liquid Medium

Alternatively, Agrobacterium can be prepared for transformation by growing in liquid medium. One day before infection, a 125-ml flask is prepared with 30 ml of 557A medium (10.5 g/l potassium phosphate dibasic, 4.5 g/l potassium phosphate monobasic anhydrous, 1 g/l ammonium sulfate, 0.5 g/l sodium citrate dehydrate, 10 g/l sucrose, 1 mM magnesium sulfate) and 30 μL spectinomycin (50 mg/mL) and 30 μL acetosyringone (20 mg/mL). A half loopful of Agrobacterium from a second plate is suspended into the flasks and placed on an orbital shaker set at 200 rpm and incubated at 28° C. overnight. The Agrobacterium culture is centrifuged at 5000 rpm for 10 min. The supernatant is removed and the Agrobacterium infection medium (700A) with acetosyringone solution is added. The bacteria is resuspended by vortex and the optical density (550 nm) of the Agrobacterium suspension is adjusted to a reading of about 0.35 to 2.0.

### D. Maize Transformation

Ears of a maize (Zea mays L.) cultivar were surface sterilized for 15-20 min in 20% (v/v) bleach (5.25% sodium hypochlorite) plus 1 drop of Tween 20 followed by 3 washes in sterile water. Immature embryos (IEs) were isolated from ears and were placed in 2 ml of Agrobacterium infection medium (700A) with acetosyringone solution. The optimal size of the embryos varied based on the inbred. The Agrobacterium infection medium (810K) was drawn off, 1 ml of the Agrobacterium suspension was added to the embryos, and the tube was vortexed for 5-10 sec. The microfuge tube was then incubated for 5 min. The Agrobacterium-embryo suspension was poured onto 7101 (or 562V) co-cultivation medium. Any residual embryos were transferred to the plate using a sterile spatula. The Agrobacterium suspension was drawn off and the embryos placed axis side down on the media. The plate was incubated in the dark at 21° C. for 1-3 days of co-cultivation. The embryos were then transferred to resting medium (605B medium) without selection.

### Example 3: Methods of In Vivo Parthenogenic Haploid Induction and Genetic Chromosome Doubling Using ZM-ODP2-(266-669)

Using the methods described in Example 2, T0 transgenic plants hemizygous for RV027603 (SEQ ID NO: 7) were obtained from a population of transformed immature diploid embryos. The immature diploid embryos had a hybrid, or first filial (F1) generation, genome. Using methods known in the art, single copy T0 plants were identified and grown to maturity. Upon the transition to flowering, the T0 plants were used as female parent plants to provide a donor ear.

The ears of the transgenic female parent plants were shoot-bagged before silk emergence to avoid any foreign pollen contamination. The silks of the donor ears were pollinated with viable pollen grains collected from the anthers of a haploid inducer parent plant, constituting a “haploid induction cross”. The paternal pollen donor possessed a stably integrated, constitutively expressing ZsYELLOW fluorescent protein color marker (YFP; data not shown) for detecting inheritance of the paternal genome caused by double fertilization and the subsequent zygotic embryogenesis of a diploid (2n) embryo.

Immature ears were harvested approximately 18 days after pollination, surface sterilized in 30% Clorox bleach plus 0.5% Micro detergent for 20 minutes and washed twice with sterile water. Immature embryos obtained from each donor ear comprising a biological replicate for a unique transformation event were plated on a petri dish containing 605B resting medium and incubated at 28° C. under dark conditions for 12-24 hours. Haploid (In) embryos possessing only maternal chromosome were detected based on the absence of paternal YFP activity and were retained; diploid embryos expressing the YFP color marker were discarded.

A total of 8 unique events for RV027603 were analyzed. Per event, two groups of haploid embryos were obtained given the hemizygous T-DNA from RV027603 inherited from the maternal plant. A first group of haploid embryos lacking inheritance of T-DNA from RV027603 was used to regenerate “null T1 plants”. A second group of haploid embryos that inherited a T-DNA from RV027603 was used to regenerate “hemizygous T1 plants”. No artificial chromosome doubling treatment, for example a chemical chromosome doubling treatment or agent, was provided to any of the haploid embryos during the experiment.

To assess the efficacy of in vivo genetic chromosome doubling in response to ZM-ODP2-(266-669) activity, the proportion of regenerated T1 plants capable of producing intact, mature seed was evaluated. The ear of each D0 (T1) plant for both the “null” or “hemizygous” T1 groups were shoot-bagged before silk emergence to avoid any foreign pollen contamination. The silks of the ears for each D0 (T1) plant were self-pollinated with pollen grains collected from the anthers of each D0 (T1) plant. After self-fertilization, the plants were grown to physiological maturity, the ears harvested, and D1 (T2) seed was counted per donor ear from each null and hemizygous plant per group per event. Seed counts for fertile plants were sorted into three categories: a low-range consisting of 1-24 kernels/ear, a medium-range consisting of 25-39 kernels/ear, and a high-range consisting of 40 or more kernels/ear.

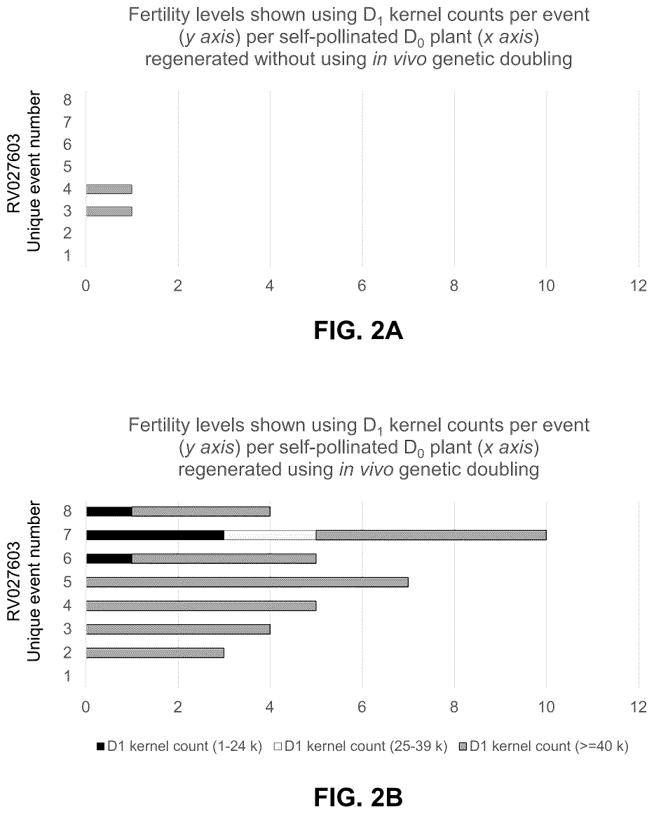

As shown in FIG. 2A, the average level of fertile hemizygous T1 plants (those having ZM-ODP2-(266-669) polypeptide activity) sampled across the 8 events was 14.9%, which was significantly greater (p<0.0053) than the average level of fertile null T1 plants samples across the same 8 events (3.17%).

These data demonstrate in vivo genetic chromosome doubling in response to ZM-ODP2-(266-669) activity achieved a level of chromosome doubling efficiency comparable to that achieved using artificial methods, such as chemical chromosome doubling methods (e.g., colchicine treatment), which has fertility rates commonly ranging from 15-20% for self-pollinated DO plants (see Frei, et. al. 2018. “The Doubled Haploid Facility”, Iowa State University Research and Demonstration Farms Progress Reports 2017(1)).

The fertility levels of the self-pollinated D0/T1 plants that produced D1/T2 kernels were further characterized (FIG. 2B). A majority of ears harvested from self-pollinated hemizygous D0/T1 plants produced 40 or more D1/T2 kernels (31 of 38 self-pollinated D0/T1 plants; 83% of the hemizygous T1 group). These data demonstrate in vivo ZM-ODP2-(266-669) activity resulted in mature, intact seeds containing diploidized maternal embryos that can produce fertile, doubled haploid plants. Such a result is consistent with in vivo chromosome doubling activity being provided to an unfertilized egg cell, for example, at the onset of parthenogenesis, or shortly thereafter, thereby resulting in a method to obtain a plant with a properly diploidized germline.

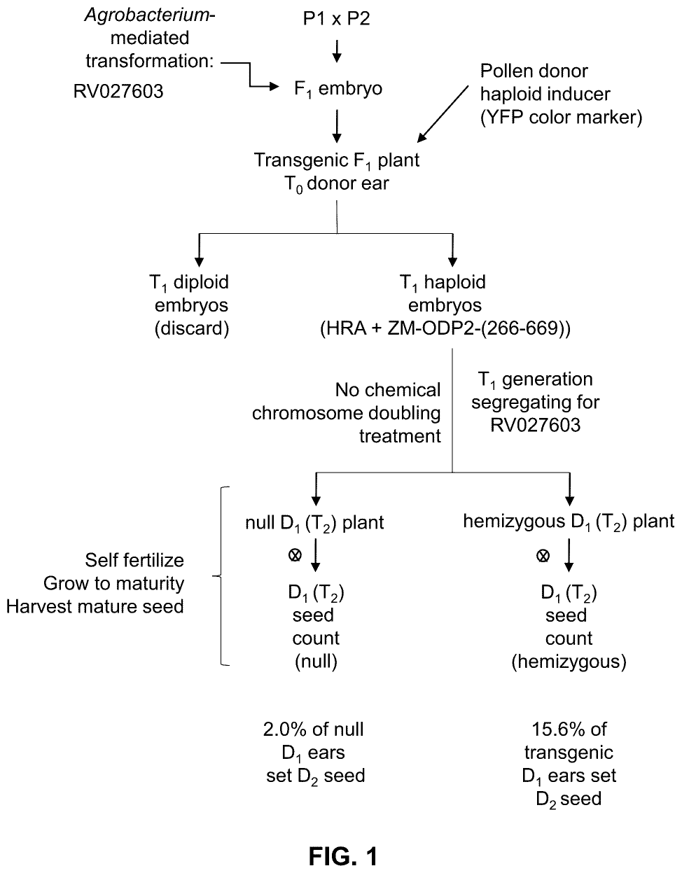

FIG. 1 illustrates a method for providing RV027603 to a diploid embryo, for example an F1 hybrid embryo, to obtain a hemizygous breeding cross. It was expected RV027603 would segregate 1:1. The transgenic F1 hybrid plant provided a T0 donor ear used for a haploid induction cross to obtain T1 generation haploid embryos lacking (null) or inheriting RV027603 (hemizygous). No artificial chromosome doubling treatment was provided to these embryos. Plants regenerated from these embryos were grown to maturity, self-fertilized (represented by a circled X), and mature seed was harvested. Restoration of fertile D1 generation plants in response to RV027603 activity within the unfertilized egg cells of the D1 generation plants is shown.

### Example 4: Methods of In Vivo Parthenogenic Haploid Induction and Genetic Chromosome Doubling Using ZM-ODP2-(266-668):At-CBF1a

In an alternative method to investigate parthenogenic haploid induction and in vivo genetic chromosome doubling in an unfertilized egg cell, a translational fusion protein comprising a heterologous, synthetic transcription factor is used.

This method uses a polynucleotide containing ZM-ODP2 (TR5)-V1 (SEQ ID NO:8) encoding the ZM-ODP2-(266-668) polypeptide (SEQ ID NO: 9) fused with a linker sequence (DNA SEQ ID NO: 10; Protein SEQ ID NO: 11) which in turn is fused to a maize optimized DNA sequence, AT-CBF1A (MO), (SEQ ID NO: 12) encoding a C-repeat/DRE binding factor domain, hereinafter “At-CBF1a” (SEQ ID NO:13). Together, this synthetic transcription factor coding sequence (SEQ ID NO: 14) encodes a ZM-ODP2-(266-668):At-CBF1a fusion protein (SEQ ID NO: 15) that is operably linked to the “PvEC1 promoter” (SEQ ID NO: 6). Using methods known in the art, RV048759 (SEQ ID NO: 16) is obtained having an expression cassette encoding the ZM-ODP2-(266-668):At-CBF1a fusion protein.

Using the methods described in Examples 2 and 3, T0 transgenic plants hemizygous for RV048759 (SEQ ID NO: 16) or RV027603 (SEQ ID NO: 7) were obtained from a population of transformed immature diploid embryos from two different genetic backgrounds. The first genetic background (hereinafter “Background 1”) was “PHR03 x PH184C” and the second genetic background (hereinafter “Background 2”) was X08D492.

The T0 transgenic plants hemizygous for RV048759 were used as female parent plants and the donor ears fertilized with pollen provided by a pollen donor. The silks of the donor ears were pollinated with viable pollen grains collected from the anthers of a haploid inducer parent plant, constituting a “haploid induction cross”. The paternal pollen donor possessed a stably integrated, constitutively expressing ZsYELLOW fluorescent protein color marker (YFP; data not shown) for detecting inheritance of the paternal genome caused by double fertilization and the subsequent zygotic embryogenesis of a diploid (2n) embryo.

A total of 13 unique events for RV048759 and a total of 7 unique events for RV027603 were analyzed in Background 1. A total of 13 unique events for RV048759 and a total of 11 unique events for RV027603 were analyzed in Background 2.

The results for haploid induction from ZM-ODP2-(266-668):At-CBF1a and ZM-ODP2-(266-669) are shown in Tables 4-7. In Background 1, haploid induction in response to ZM-ODP2-(266-668):At-CBF1a ranged from 9.9%-44.4%, with an average haploid induction rate of 31.3%. By comparison, haploid induction in response to ZM-ODP2-(266-669) ranged from 0.6%-7.0% in the same background, with an average haploid induction rate of 3.5%. In Background 2, haploid induction in response to ZM-ODP2-(266-668):At-CBF1a ranged from 16.3%-42.6%, with an average haploid induction rate of 33.9%. By comparison, haploid induction in response to ZM-ODP2-(266-669) ranged from 2.4%-25.8% in the same background, with an average haploid induction rate of 15.1%.

Collectively, these data suggest At-CBF1a improves the haploid induction rate of ZM-ODP2-(266-668).

TABLE 4
Haploid Induction of ZM-ODP2-(266-668):At-CBF1a in Background 1
ZM-ODP2-(266-668):At-CBF1a (RV048759) in Background 1

| | Total | Haploid | Haploid Induction |
|---|---|---|---|
| Event ID | Embryos | Embryos | Rate (%) |
| Event 1 | 536 | 79 | 14.7 |
| Event 2 | 580 | 237 | 40.9 |
| Event 3 | 414 | 125 | 30.2 |
| Event 4 | 558 | 231 | 41.4 |
| Event 5 | 449 | 192 | 42.8 |
| Event 6 | 372 | 165 | 44.4 |
| Event 7 | 349 | 72 | 20.6 |
| Event 8 | 172 | 17 | 9.9 |
| Event 9 | 448 | 174 | 38.8 |
| Event 10 | 362 | 141 | 39.0 |
| Event 11 | 258 | 75 | 29.1 |
| Event 12 | 305 | 54 | 17.7 |
| Event 13 | 184 | 68 | 37.0 |

TABLE 5
Haploid Induction of ZM-ODP2-(266-669) in Background 1
ZM-ODP2-(266-669) (RV027603) in Background 1

| | Total | Haploid | Haploid Induction |
|---|---|---|---|
| Event ID | Embryos | Embryos | Rate (%) |
| Event 1 | 500 | 3 | 0.6 |
| Event 2 | 642 | 32 | 5.0 |
| Event 3 | 313 | 22 | 7.0 |
| Event 4 | 323 | 8 | 2.5 |
| Event 5 | 329 | 8 | 2.4 |
| Event 6 | 671 | 13 | 1.9 |
| Event 7 | 691 | 33 | 4.8 |

TABLE 6
Haploid Induction of ZM-ODP2-(266-668):At-CBF1a in Background 2
ZM-ODP2-(266-668):At-CBF1a (RV048759) in Background 2

| | Total | Haploid | Haploid Induction |
|---|---|---|---|
| Event ID | Embryos | Embryos | Rate (%) |
| Event 1 | 356 | 91 | 25.6 |
| Event 2 | 333 | 142 | 42.6 |
| Event 3 | 289 | 104 | 36.0 |
| Event 4 | 160 | 26 | 16.3 |
| Event 5 | 413 | 138 | 33.4 |
| Event 6 | 245 | 99 | 40.4 |
| Event 7 | 163 | 61 | 37.4 |
| Event 8 | 441 | 109 | 24.7 |
| Event 9 | 499 | 161 | 32.3 |
| Event 10 | 279 | 108 | 38.7 |
| Event 11 | 371 | 150 | 40.4 |
| Event 12 | 387 | 147 | 38.0 |
| Event 13 | 396 | 139 | 35.1 |

TABLE 7
Haploid Induction of ZM-ODP2-(266-669) in Background 2
ZM-ODP2-(266-669) in Background 2

| | Total | Haploid | Haploid Induction |
|---|---|---|---|
| Event ID | Embryos | Embryos | Rate (%) |
| Event 1 | 149 | 6 | 4.0 |
| Event 2 | 366 | 60 | 16.4 |
| Event 3 | 303 | 40 | 13.2 |
| Event 4 | 176 | 16 | 9.1 |
| Event 5 | 358 | 74 | 20.7 |
| Event 6 | 220 | 44 | 20.0 |
| Event 7 | 163 | 42 | 25.8 |
| Event 8 | 208 | 5 | 2.4 |
| Event 9 | 257 | 60 | 23.3 |
| Event 10 | 169 | 19 | 11.2 |
| Event 11 | 297 | 59 | 19.9 |

### Example 5: Methods of In Vivo Parthenogenic Haploid Induction and Genetic Chromosome Doubling Using a Cell Non-Autonomous ZM-ODP2 Fusion Polypeptide

An unexpected finding demonstrated in Example 3 was that 3.2% of the null T1 plants produced mature, intact seed in response to self-pollination. It is hypothesized this result was caused by the ZM-ODP2-(266-669) polypeptide acting as a morphogen with a concentration gradient affecting adjacent cells. More specifically, the ZM-ODP2-(266-669) polypeptide expressed by a sporophytic, diploid (2n) cell hemizygous for the T-DNA of RV027603 acted as a morphogen affecting maternal cells, such as the embryo sac, the unfertilized egg cell, and/or an unfertilized haploid egg cell lacking the T-DNA of RV027603. In this manner, a concentration gradient formed an embryogenic condition sufficient to induce both haploid induction and in vivo chromosome doubling within a null-segregant egg cell such that chromosome doubling and fertility was sufficiently restored in 3.2% of the null T1 plants regenerated from the embryos contacted with the ZM-ODP2-(266-669) polypeptide. This level of restored fertility to a null embryo was greater than expected in response to spontaneous chromosomal doubling, and thus, demonstrated a novel improvement to the state of the art.

A cell non-autonomous fusion polypeptide comprises a polynucleotide containing a ZM-ODP2 (TR5)-V1 sequence (SEQ ID NO: 8), a linker sequence (SEQ ID MP: 10), and cell penetrating polypeptide (CPP) sequence, for example a KNOTTED-1 CPP DNA fragment (DNA SEQ ID NO: 38, Protein SEQ ID NO: 39) encoding a ZM-ODP2-(266-668):KNOTTED-1 CPP fusion polypeptide (DNA SEQ ID NO: 40; Protein SEQ ID NO: 41).

To provide the fusion polypeptide as a solute from a non-reduced, diploid sporophytic cell hemizygous for the T-DNA containing the expression cassette to a reduced, haploid gametic cell, the fusion polypeptide is expressed in the transfer cells located in the basal endosperm. In this experiment, the ZM-ODP2-(266-668):KNOTTED-1 CPP coding sequence is operably linked to a regulatory element with tissue-preferred activity in maize basal endosperm transfer layer (BETL), for example, a BETL9 regulatory element (SEQ ID NO: 42).

The ZM-ODP2-(266-668):KNOTTED-1 CPP coding sequence further comprises a polynucleotide containing a DNA fragment encoding a BETL secretion peptide, for example the BETL9 secretion polypeptide (DNA SEQ ID NO: 43, Protein SEQ ID NO: 44). The BETL9 secretion polypeptide is designed to be fused to the N-terminus, the C-terminus, or alternatively with a copy on the N-terminus and the C-terminus of the ZM-ODP2-(266-668):KNOTTED-1 CPP translational fusion peptide.

To investigate transfer of non-reduced, diploid transfer cells located in the basal endosperm to a reduced, haploid cell, the translational fusion polypeptide further comprises a DNA sequence encoding a polypeptide useful for demonstrating transfer of a translational fusion product. The KNOTTED-1 CPP translational fusion polypeptide is a ZM-ODP2-(266-668):KNOTTED-1 CPP fusion polypeptide (DNA SEQ ID NO: 40, Protein SEQ ID no: 41) containing a BETL9 secretion polypeptide (DNA SEQ ID NO: 43, Protein SEQ ID NO: 44), and more specifically contains a green fluorescent protein from Aequorea coerulescens, herein called “AC-GFP1” (DNA SEQ ID NO: 45, Protein SEQ ID NO: 46).

Using the methods described in Examples 2 and 3, T0 transgenic plants hemizygous for a plasmid containing a ZM-ODP2-(266-668):KNOTTED-1 CPP expression cassette (SEQ ID NO: 40) operably linked to BETL9 regulatory element (SEQ ID NO: 42) are produced. Such hemizygous plants can alternatively express the ZM-ODP2-(266-668):KNOTTED-1 CPP further comprising a BETL9 secretion peptide, or a translation fusion polypeptide encoding a BETL9 secretion polypeptide fused to the ZM-ODP2-(266-668):KNOTTED-1 CPP polypeptide fused to a AC-GFP1 polypeptide as described above.

Post transformation, each regenerated plant that is a hemizygous T₀ plant having one stably inserted copy of a T-DNA is considered a unique event and grown to maturity. The ears of each hemizygous T₀ plant are shoot-bagged before silk emergence to avoid any foreign pollen contamination. The silks of the ears on the plants of the female parent plants are pollinated with viable pollen grains. Such pollen grains are collected from the anthers of a male non-haploid inducer parent plant constitutively expressing a cyan fluorescent protein color marker (CFP). Alternatively, the pollen grains can be collected from the anthers of a male haploid inducer parent plant, preferentially, the pollen donor is a haploid inducer lines, such as Stock 6, RWS, KEMS, KMS, ZMS, or related derivatives thereof, used as a male parent plant. Such a haploid inducer parent plant constitutively expresses a paternal color marker, such as a yellow fluorescent protein color marker (YFP), an anthocyanin color marker, or both.

After fertilization, mature seed is harvested and scored for the presence/absence of the paternal color marker. A seed expressing the paternal reporter gene is discarded; a seed without said paternal activity is retained.

A seed without said paternal activity is sampled in a non-destructive manner for the purpose of DNA isolation. The isolated DNA is used for a diagnostic PCR-based assay to detect presence/absence of any transgene, including determining the copy number of any transgenic construct, and for genotyping using PCR-based genetic marker assay methods known in the art. The genotypic data is used to determine allelic states inherited at genome-wide marker loci useful for breeding selection methods known in the art, such as an estimate to calculate genomic estimated breeding values (GEBVs) for individuals without first having phenotypic data. Such genomic estimated breeding values (GEBVs) provide a method for identifying doubled haploid progeny lacking desirable genotypes that are discarded and doubled haploid progeny possessing desirable genotypes to retain, thereby providing a method to enrich the structure of a doubled haploid population to be optimized for breeding purposes.

### Example 6: Methods of In Vitro Somatic Embryogenesis Induction and Genetic Chromosome Doubling Using ZM-ODP2-(266-669)

The following experiment investigates in vitro somatic embryogenesis induction and chromosome doubling in a maize haploid embryo in vitro to obtain doubled haploid plants.

### Example 6A: Method to Obtain a Genetically Diverse Population of Maize Haploid Embryos

Seeds from an F1 hybrid maize plant resulting from cross fertilization of two genetically different inbred parental strains were planted, and F1 hybrid plants were used as female parent plants (pollen receivers). Genetic diversity is created per ovule, with each ovule producing a genetically unique egg cell due to meiotic recombination during megagametogenesis. Seeds from haploid inducer lines, such as Stock 6, RWS, KEMS, KMS, ZMS, or related derivatives were grown, and the resulting plants were used as male parent plants (pollen donors). The ears of the female parent plants were shoot-bagged before silk emergence. The silks of the ears on the plants of the female parent plants were pollinated with viable pollen grains collected from the anthers of the male parent plants (haploid inducer plants). This pollination was controlled by the method used regularly in maize breeding programs to avoid foreign pollen contamination.

Approximately 9-14 days after pollination, immature ears were harvested. The ears were surface sterilized in 30% Clorox bleach plus 0.5% Micro detergent for 20 minutes, washed twice with sterile water, and dissected to obtain immature embryos from each ear.

The haploid embryos were selected based on the absence of a paternal reporter gene. For example, if the paternal inducer line is stably transformed with a fluorescent reporter trait gene or has an anthocyanin reporter gene, for example the R1-scm gene (see U.S. Pat. No. 8,859,846 incorporated herein by reference in its entirety), then such gene activity can be used at an early developmental stage for ploidy determination. After paternal genome elimination, maternal haploid embryos having only one set of chromosomes from the female parent in the embryo cells are scored as haploid embryos based on the absence of the paternal reporter gene.

This method results in the production of about 2-30% of all embryos being haploid embryos per ear, with frequencies known to differ per choice of the haploid inducer line used. In this experiment, diploid embryos were discarded; haploid embryos were retained for use in the following steps.

### Example 6B: Inducing Simultaneous Somatic Embryogenesis and Chromosome Doubling In Vitro

Haploid embryos from Section A are split into three groups, each group receiving a treatment as shown in Table 8.

TABLE 8
Treatment Groups

| | First | Second |
|---|---|---|
| Treatment | Agrobacterium strain | Agrobacterium strain |
| 1 | RV020636 | RV047438 |
| 2 | RV020636 | RV047990 |
| 3 | RV020636 | RV047991 |

In this experiment, two Agrobacterium LBA4404 THY− strains (See U.S. Pat. No. 8,334,429 incorporated herein by reference in its entirety) are used in a mixture to co-transform each group of embryos. The first Agrobacterium strain shared with all three treatment groups is a strain with RV020636 (SEQ ID NO: 19) which contains a WUS expression cassette operably linked the Zea mays PLTP regulatory element with a maize-derived enhancer modulating element (EME) used in triplicate (+3XEME) positioned near the PLTP promoter TATA box. PHP88156 is useful for contacting a first plant cell with its T-DNA, where the WUS expression cassette provides WUS protein activity that can elicit a growth response in a second plant cell, such as a somatic embryogenesis response. This method is particularly useful when the second plant cell lacks the T-DNA from RV020636. RV020636 is not known to affect chromosome doubling.

For treatment 1, the second Agrobacterium strain with RV047438 (SEQ ID NO: 20) lacks a ZM-ODP2-(266-669) expression cassette and is a negative control. For treatment 2, the second Agrobacterium strain with RV047990 (SEQ ID NO: 21) contains the ZM-ODP2 (TR5) (SEQ ID NO: 1) operably linked to a ubiquitin regulatory element (SEQ ID NO: 17) comprising the UBI1ZM PRO, UBI1ZM 5UTR (PHI), and UBI1ZM INTRON1 (PHI) feature elements. For treatment 3, the second Agrobacterium strain with RV047991 (SEQ ID NO: 22) contains the ZM-ODP2 (TR5) (SEQ ID NO: 1) operably linked to a maize PLTP (phospholipid transfer protein) regulatory element (SEQ ID NO: 18) comprising the ZM-PLTP PRO and ZM-PLTP 5 UTR feature elements. For each treatment, a mixture comprising 10% of the first Agrobacterium with RV020636 and 90% of the second Agrobacterium comprising each respective plasmid is used.

Transformation for each group is performed as described in Example 2 to induce somatic embryogenesis. After 6-10 days, the proliferating tissue of each group is dissected, each portion of dissected tissue is transferred to maturation medium (289Q) and cultured at 26-28° C. under dark conditions. After approximately 6-10 days the sub-cultured tissue is transferred to a light culture room at 26° C. until healthy plantlets with roots develop. Approximately 7-14 days later, plantlets are transferred to flats containing potting soil, and grown for 1 week in a growth chamber, subsequently grown an additional 1-2 weeks in the greenhouse, and then transplanted to soil in pots and grown under greenhouse conditions.

A leaf tissue sample is collected from each regenerated plant, DNA is isolated, and a diagnostic PCR-based assay is performed to detect presence/absence of each T-DNA using methods known in the art. The isolated DNA can also be used for genotyping using PCR-based genetic marker assay methods known in the art. Such genotypic data can be used for a variety of known breeding methods, including but not limited to marker assisted selection and whole genome prediction methods.

### Example 6C: Inducing Simultaneous Genome Modification, Somatic Embryogenesis, and Chromosome Doubling In Vitro

The following experiment investigates obtaining a genome-modified doubled haploid plant from a haploid embryo using an in vitro treatment lacking an artificial, chemical chromosome doubling agent. Immature haploid embryos of a maize F1 hybrid crossed with haploid inducer are obtained as described in Section A and split into two groups for treatment as described below.

The two treatments each comprise a mixture of Agrobacterium LBA4404 THY-strains (See U.S. Pat. No. 8,334,429 incorporated herein by reference in its entirety) to co-transform each group of haploid embryos.

Each treatment uses a first Agrobacterium strain with RV020636 (SEQ ID NO: 19) containing a WUS expression cassette operably linked the maize PLTP (phospholipid transfer protein) regulatory element with a maize-derived enhancer modulating element (EME) used in triplicate (+3XEME) positioned near the PLTP promoter TATA box. RV020636 is useful for contacting a first plant cell with its T-DNA, where the WUS expression cassette provides WUS protein activity that can elicit a growth response in a second plant cell, such as a somatic embryogenesis response. This method is particularly useful when the second plant cell lacks the T-DNA from RV020636. RV020636 is not known to affect chromosome doubling.

Each treatment uses a unique second Agrobacterium strain. The first treatment uses RV048128 (SEQ ID NO: 23) containing, i.) a genome modification expression cassette, ii.) a dual gRNA expression cassette targeting the Zea mays NAC7 locus (SEQ ID: 25), iii.) a ZM-ODP2 (TR5) (SEQ ID: 1) expression cassette operably linked to a ubiquitin regulatory element (SEQ ID NO: 17) comprising the UBI1ZM PRO, UBI1ZM 5UTR (PHI), and UBI1ZM INTRON1 (PHI) feature elements. The second treatment uses RV048055 (SEQ ID NO: 24) containing, i.) a genome modification expression cassette, ii.) a dual gRNA expression cassette targeting a Zea mays NAC7 locus (SEQ ID: 25), iii.) a ZM-ODP2 (TR5) (SEQ ID NO: 1) expression cassette operably linked to a maize PLTP (phospholipid transfer protein) regulatory element (SEQ ID NO: 18) comprising the ZM-PLTP PRO and ZM-PLTP 5 UTR feature elements. For each treatment, a mixture comprising 10% of the first Agrobacterium with RV020636 and 90% of the second Agrobacterium strain containing each respective plasmid is used.

Transformation steps for each treated group are performed as described in Example 2. After approximately 6-10 days the proliferating tissue of each group is dissected, each portion of dissected tissue is transferred to maturation medium (289Q) and cultured at 26-28° C. under dark conditions. After 6-10 days the sub-cultured tissues are transferred to a light culture room at 26° C. until healthy plantlets with roots develop. Approximately 7-14 days later, plantlets are transferred to flats containing potting soil and grown for 1 week in a growth chamber, subsequently grown an additional 1-2 weeks in the greenhouse, and then transplanted to soil in pots and grown under greenhouse conditions.

### Example 7: Methods of In Vivo Parthenogenic Haploid Induction, Genetic Chromosome Doubling, and Genome Modification Using ZM-ODP2-(266-669)

This experiment used a non-haploid inducer line that was a F1 hybrid obtained by cross-fertilization of two parental lines. Approximately 10 days after pollination, an immature, diploid F1 embryo was transformed using the methods described in Examples 2 and 3. Simultaneous genome modification, parthenogenic haploid induction, and in vivo chromosome doubling activities was performed using RV034410 (SEQ ID NO: 47) containing a T-DNA capable of regenerating a F1 hybrid T0 plant with i.) a ZM-ODP2-(266-669) expression cassette for conferring parthenogenic haploid induction and in vivo chromosome doubling activities, ii.) a Cas9 gene editing expression cassette for creating a double strand break, iii) a dual guide RNA (gRNA) expression cassette for conferring targeted genome modification, iv.) a DsRED color marker expression cassette useful as a reporter gene product for detecting presence/absence of T-DNA integration, and v.) a maize optimized (MO) CRE recombinase expression cassette for excising the polynucleotide sequence intervening the two loxP sites within the plasmid sequence.

Post transformation, each regenerated plant that was a hemizygous T₀ (F1 hybrid) plant having one copy of a T-DNA was considered a unique event that was grown to maturity. The ears of each hemizygous T₀ plant were shoot-bagged before silk emergence to avoid any foreign pollen contamination. The silks of the ears on the plants of the female parent plants were pollinated with viable pollen grains. Such pollen grains were collected from the anthers of a male non-haploid inducer parent plant constitutively expressing a cyan fluorescent protein color marker (CFP).

At 9-21 days after pollination, preferentially at 18 days, the immature ears were harvested. The ears were surface sterilized in 30% Clorox bleach plus 0.5% Micro detergent for 20 minutes and washed twice with sterile water. Embryos were isolated using a scalpel and placed on a medium lacking an artificial, chemical chromosome doubling agent, at 28° C. under dark conditions for 12-16 hours. The embryos were scored for the presence/absence of the paternal marker gene inherited from the non-inducer paternal parent. Here, haploid embryos without CFP were retained, while diploid embryos with CFP activity were discarded. The CFP-negative embryos were then cultured under the same conditions and after approximately 6-10 days each plantlet was transferred to a light culture room.

For RV034410, five unique events were sampled resulting in a total of 27 CFP-negative DO embryos that were obtained. These embryos were cultured to regenerate green leaf tissue that were sampled for flow cytometry analysis. Briefly, flow cytometry analysis of ploidy involves staining nuclei from cells with a fluorescent dye that binds to DNA and analyzes samples using a histogram plot showing ploidy patterns based on DNA content. Using this method, patterns associated with haploid or diploid patterns per leaf sample per event were obtained.

Five unique events of plants transformed with RV034410 were sampled and analyzed using flow cytometry analysis for ploidy determination; results are shown in Table 9.

TABLE 9
Ploidy Determination

| RV034410 | CFP-negative D₀ | Haploid | Diploid | Diploid |
|---|---|---|---|---|
| Events | embryo total | (count) | (count) | % |
| Event 1 | 1 | 1 | 0 | 0% |
| Event 2 | 3 | 2 | 1 | 33% |
| Event 3 | 3 | 2 | 1 | 33% |
| Event 4 | 14 | 8 | 6 | 43% |
| Event 5 | 6 | 2 | 4 | 67% |
| Average | | | | 35% |
| Total | 27 | 15 | 12 | |

For these events, 35% of the sampled CFP-embryos that were regenerated into plants exhibited flow cytometry patterns consistent with in vivo chromosome doubling.

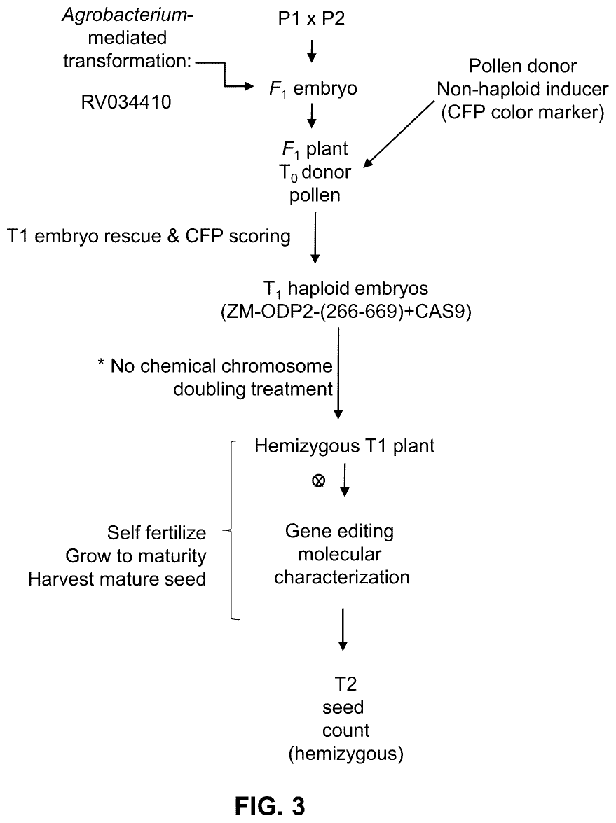

FIG. 3 illustrates a method for providing RV034410 to a diploid embryo, for example an F1 hybrid embryo, to obtain a hemizygous breeding cross. It was expected RV034410 would segregate 1:1. The transgenic F1 hybrid plant provided a T0 donor ear used for a haploid induction cross to obtain T1 generation haploid embryos. Embryos inheriting RV034410 (hemizygous) were regenerated into T1 plants. No artificial chromosome doubling treatment was provided to these embryos. T1 generation haploid embryos were regenerated into plants, analyzed for genome modification at the genomic target site, and grown to maturity to obtain mature, intact T2 generation seed comprising genome-modified, maternally derived doubled haploids.

For haploid induction and genome modification, a total of 7 unique events were analyzed in Background 2. The results for haploid induction and genome modification are shown in Tables 10a and 10b.

TABLE 10a
Haploid Induction of ZM-ODP2-(266-669) +
Genome Editing + Cre Excision

| | | | Haploid | Plant | |
|---|---|---|---|---|---|
| | Total | Haploid | Induction | with Cre | Excision |
| Event ID | Embryos | Plants | Rate (%) | Excision | (%) |
| Event 1 | 1553 | 24 | 1.5 | 14 | 58.3 |
| Event 2 | 3870 | 37 | 1.0 | 21 | 56.8 |
| Event 3 | 3629 | 48 | 1.3 | 25 | 52.1 |
| Event 4 | 2993 | 27 | 0.9 | 16 | 59.3 |
| Event 5 | 454 | 1 | 0.2 | 1 | 100.0 |
| Event 6 | 1564 | 26 | 1.7 | 17 | 65.4 |
| Event 7 | 2141 | 41 | 1.9 | 26 | 63.4 |
| Average | | | 1.2 | | 65.0 |
| Total | 16204 | 204 | | 120 | |

TABLE 10b
Genome Editing of ZM-ODP2-(266-669) +
Genome Editing + Cre Excision

| | | | | Edited | Edited |
|---|---|---|---|---|---|
| | Plant | Total | Edited | Plants | Plants |
| | Returning | Edited | Plants | with Cre | with Cre |
| Event ID | GEMC* | Plants | (%) | Excision | Excision (%) |
| Event 1 | 23 | 23 | 100.0 | 14 | 60.9 |
| Event 2 | 37 | 34 | 91.9 | 20 | 54.1 |
| Event 3 | 48 | 45 | 93.8 | 22 | 45.8 |
| Event 4 | 27 | 26 | 96.3 | 15 | 55.6 |
| Event 5 | 1 | 1 | 100.0 | 1 | 100.0 |
| Event 6 | 25 | 24 | 96.0 | 16 | 64.0 |
| Event 7 | 40 | 39 | 97.5 | 25 | 62.5 |
| Average | | | 96.5 | | 63.3 |
| Total | 201 | 192 | | 113 | |

*GEMC = gene editing molecular characterization

These data demonstrate it was possible to obtain a maternal embryo that was diploidized in vivo. Importantly, this outcome was achieved without performing in vitro tissue culture of an immature haploid embryo, nor was this performed applying a artificial chromosome doubling treatment, for example, a chemical chromosome doubling treatments, such as colchicine. Furthermore, it was possible to obtain such mature seed wherein Cre-mediated excision had occurred.

Given the result shown in Example 3, it is anticipated the current method will enable obtaining mature seed having a maternally derived embryo containing a gene edited, diploid (or “di-haploid”) maternal genome. It is expected that a proportion of the seed will furthermore have maternally derived embryo containing a gene edited, diploid (or “di-haploid”) maternal genome, wherein Cre-mediated excision will enable obtaining a mature seed having a gene edited, di-haploid embryo having an excised T-DNA.

### Example 8: Methods of In Vivo Parthenogenic Haploid Induction, Genetic Chromosome Doubling, and Genome Modification Using ZM-ODP2-(266-668):AtCBF1a Translation Fusion

This experiment uses a non-haploid inducer line that was an F1 hybrid obtained by cross-fertilization of two parental lines. Approximately 10 days after pollination, an immature, diploid F1 embryo is transformed using the methods described in Examples 2 and 3. Simultaneous genome modification, parthenogenic haploid induction, and in vivo chromosome doubling activities is performed using RV053058 (SEQ ID NO: 48) containing a T-DNA capable of regenerating a F1 hybrid T0 plant with i.) a synthetic transcription factor coding sequence (SEQ ID NO: 14) that encodes a ZM-ODP2-(266-668):At-CBF1a fusion protein (SEQ ID NO: 15) that is operably linked to the PvEC1 promoter (SEQ ID NO: 6) for conferring parthenogenic haploid induction and in vivo chromosome doubling activities, ii.) a Cas9 gene editing expression cassette for creating a double strand break, iii) a dual guide RNA (gRNA) expression cassette for conferring targeted genome modification, iv.) a DsRED color marker expression cassette useful as a reporter gene product for detecting presence/absence of T-DNA integration, and v.) a maize optimized (MO) CRE recombinase expression cassette for excising the polynucleotide sequence intervening the two loxP sites within the plasmid sequence.

Post transformation, each regenerated plant that is a hemizygous T₀ (F1 hybrid) plant having one copy of a T-DNA is considered a unique event that is transplanted to soil for growth to maturity. The ears of each hemizygous T₀ plant are shoot-bagged before silk emergence to avoid any foreign pollen contamination. The silks of the ears on the plants of the female parent plants are pollinated with viable pollen grains. Such pollen grains are collected from the anthers of a male non-haploid inducer parent plant constitutively expressing a cyan fluorescent protein color marker (CFP).

It is expected the current method using RV053058 will be an improvement in comparison to the results shown in Example 7.

### Example 9: In Vivo Genome Editing Method Using ZM-ODP2-(266-669) and a Cyclin Gene

The following experiment utilized an expression cassette for conferring parthenogenic haploid induction and in vivo chromosome doubling activity here conferred by two proteins, here using gene products of both the ZM-ODP2-(266-669) and cyclin proteins.

Simultaneous genome modification, parthenogenic haploid induction, and in vivo chromosome doubling activities was performed using RV048819 (SEQ ID NO: 51) containing a T-DNA capable of regenerating a F1 hybrid T0 plant with i.) a ZM-ODP2-(266-669) expression cassette for conferring parthenogenic haploid induction and in vivo chromosome doubling activities, ii.) a Cas9 gene editing expression cassette for creating a double strand break, iii) a dual guide RNA (gRNA) expression cassette for conferring targeted genome modification, iv.) a DsRED color marker expression cassette useful as a reporter gene product for detecting presence/absence of T-DNA integration, v.) a maize optimized (MO) CRE recombinase expression cassette for excising the polynucleotide sequence intervening the two loxP sites within the plasmid sequence, and vi) a polynucleotide containing a ZM-CYCD2 sequence (DNA SEQ ID NO: 49, Protein SEQ ID NO: 50) operably linked to a to the PvEC1 promoter (SEQ ID NO: 6).

This experiment used a non-haploid inducer line that was a F1 hybrid obtained by cross-fertilization of two parental lines. Approximately 10 days after pollination, an immature, diploid F1 embryo was transformed using the methods described in Examples 2 and 3 and the phenotypic analysis was performed as described in Example 8.

The results for haploid induction and genome modification are shown in Tables 11a and 11b. A total of 23 unique events for RV048819 were analyzed. Results are shown in Tables 11a and 11b.

TABLE 11a
Haploid Induction of ZM-ODP2-(266-669) + Cyclin Gene +
Genome Editing + Cre Excision

| | | | Haploid | Plant | |
|---|---|---|---|---|---|
| | Total | Haploid | Induction | with Cre | Excision |
| Event ID | Embryos | Plants | Rate (%) | Excision | (%) |
| Event 1 | 373 | 2 | 0.5 | 1 | 50.0 |
| Event 2 | 253 | 3 | 1.2 | 0 | 0.0 |
| Event 3 | 421 | 1 | 0.2 | 1 | 100.0 |
| Event 4 | 534 | 9 | 1.7 | 7 | 778 |
| Event 5 | 485 | 4 | 0.8 | 3 | 75.0 |
| Event 6 | 430 | 1 | 0.2 | 1 | 100.0 |
| Event 7 | 463 | 1 | 0.2 | 0 | 0.0 |
| Event 8 | 431 | 2 | 0.5 | 2 | 100.0 |
| Event 9 | 385 | 2 | 0.5 | 1 | 50.0 |
| Event 10 | 373 | 1 | 0.3 | 0 | 0.0 |
| Event 11 | 407 | 0 | — | — | — |
| Event 12 | 139 | 0 | — | — | — |
| Event 13 | 400 | 0 | — | — | — |
| Event 14 | 368 | 0 | — | — | — |
| Event 15 | 273 | 3 | 1.1 | 0 | 0.0 |
| Event 16 | 346 | 3 | 0.9 | 3 | 100.0 |
| Event 17 | 347 | 1 | 0.3 | 0 | 0.0 |
| Event 18 | 583 | 1 | 0.2 | 0 | 0.0 |
| Event 19 | 235 | 1 | 0.4 | 1 | 100.0 |
| Event 20 | 513 | 2 | 0.4 | 0 | 0.0 |
| Event 21 | 443 | 2 | 0.5 | 2 | 100.0 |
| Event 22 | 424 | 3 | 0.7 | 1 | 33.3 |
| Event 23 | 475 | 1 | 0.2 | 0 | 0.0 |
| Average | | | 0.5 | | 46.6 |
| Total | 9101 | 43 | | 12 | |

TABLE 11b
Genome Editing of ZM-ODP2-(266-669) + Cyclin Gene +
Genome Editing + Cre Excision

| | | | | Edited | Edited |
|---|---|---|---|---|---|
| | Plant | Total | Edited | Plants | Plants |
| | Returning | Edited | Plants | with Cre | with Cre |
| Event ID | GEMC | Plants | (%) | Excision | Excision (%) |
| Event 1 | 2 | 1 | 50.0 | 1 | 50.0 |
| Event 2 | 3 | 3 | 100.0 | 0 | 0.0 |
| Event 3 | 1 | 1 | 100.0 | 1 | 100.0 |
| Event 4 | 9 | 9 | 100.0 | 7 | 77.8 |
| Event 5 | 4 | 3 | 75.0 | 2 | 50.0 |
| Event 6 | 1 | 1 | 100.0 | 1 | 100.0 |
| Event 7 | 1 | 1 | 100.0 | 0 | 0.0 |
| Event 8 | 2 | 1 | 50.0 | 1 | 50.0 |
| Event 9 | 2 | 2 | 100.0 | 1 | 50.0 |
| Event 10 | 1 | 1 | 100.0 | 0 | 0.0 |
| Event 11 | — | — | — | — | — |
| Event 12 | — | — | — | — | — |
| Event 13 | — | — | — | — | — |
| Event 14 | — | — | — | — | — |
| Event 15 | 3 | 3 | 100.0 | 0 | 0.0 |
| Event 16 | 3 | 3 | 100.0 | 3 | 100.0 |
| Event 17 | 1 | 1 | 100.0 | 0 | 0.0 |
| Event 18 | 1 | 1 | 100.0 | 0 | 0.0 |
| Event 19 | 1 | 1 | 100.0 | 1 | 100.0 |
| Event 20 | 2 | 2 | 100.0 | 0 | 0.0 |
| Event 21 | 2 | 2 | 100.0 | 2 | 100.0 |
| Event 22 | 3 | 3 | 100.0 | 1 | 33.3 |
| Event 23 | 1 | 1 | 100.0 | 0 | 0.0 |
| Average | | | 93.4 | | 42.7 |
| Total | 43 | 40 | | 21 | |

These data demonstrate it was possible to obtain a maternal embryo that was diploidized in vivo. However, it was unexpected that in vivo chromosome doubling activity using two proteins (e.g. ZM-ODP2-(266-669)+cyclin) as performed here would decrease the haploid induction rate from 1.2% as shown in Example 8 to the 0.5% level shown here. Furthermore, the current method resulted in a relatively lower recovery of progeny having Cre-mediated excision (46.6%; Table 11a) and edited plants having Cre-mediated excision (42.7%; Table 11b).

### Example 10: In Vivo Genome Editing Method Using a ZM-ODP2-(266-668):AtCBF1a Translation Fusion and a Cyclin Gene

The following experiment utilized an expression cassette for conferring parthenogenic haploid induction and in vivo chromosome doubling activities using polynucleotide encoding a translational fusion protein as described in Examples 4 and 8.

The method used plasmid RV048931 (SEQ ID NO: 52) containing a polynucleotide containing ZM-ODP2 (TR5)-V1 (SEQ ID NO: 8) encoding the ZM-ODP2-(266-668) polypeptide (SEQ ID NO: 9) fused with a linker sequence (DNA SEQ ID NO: 10; Protein SEQ ID NO: 11) which in turn is fused to a maize optimized DNA sequence, AT-CBF1A (MO), (SEQ ID NO: 12) encoding a C-repeat/DRE binding factor domain (At-CBF1a) (SEQ ID NO:13). Together, this synthetic transcription factor coding sequence (SEQ ID NO: 14) encodes a ZM-ODP2-(266-668):At-CBF1a fusion protein (SEQ ID NO: 15) that is operably linked to the PvEC1 promoter (SEQ ID NO: 6). Further, plasmid RV048931 contains a polynucleotide encoding a cyclin gene used as a genetic chromosome doubling agent in combination with the ZM-ODP2-(266-668):At-CBF1a fusion protein. A polynucleotide containing a ZM-CYCD2 sequence (DNA SEQ ID NO: 49, Protein SEQ ID NO: 50) operably linked to a to the PvEC1 promoter (SEQ ID NO: 6) was used. Additionally, plasmid RV048931 contains the gene editing components as described in Example 9.

T0 transgenic plants hemizygous for RV048931 were obtained from a population of transformed immature diploid embryos. The T0 transgenic plants hemizygous for RV048931 were used as female parent plants and the donor ears fertilized with pollen provided by a pollen donor. The paternal pollen donor possessed a stably integrated, constitutively expressing ZsYELLOW fluorescent protein color marker for detecting inheritance of the paternal genome caused by double fertilization and the subsequent zygotic embryogenesis of a diploid (2n) embryo (Example 3).

A total of 11 unique events for RV0276030 (ZM-ODP2-(266-668):At-CBF1a) were analyzed. Results are shown in Tables 12a and 12b.

TABLE 12a
Haploid Induction of ZM-ODP2-(266-668):AtCBF1a + Cyclin
Gene + Genome Editing + Cre Excision

| | | | Haploid | Plant | |
|---|---|---|---|---|---|
| | Total | Haploid | Induction | with Cre | Excision |
| Event ID | Embryos | Plants | Rate (%) | Excision | (%) |
| Event 1 | 420 | 0 | 0.0 | — | — |
| Event 2 | 414 | 10 | 2.4 | 5 | 50.0 |
| Event 3 | 408 | 11 | 2.7 | 7 | 63.6 |
| Event 4 | 340 | 11 | 3.2 | 7 | 63.6 |
| Event 5 | 358 | 16 | 4.5 | 13 | 81.3 |
| Event 6 | 260 | 8 | 3.1 | 6 | 75.0 |
| Event 7 | 362 | 0 | 0.0 | — | — |
| Event 8 | 336 | 15 | 4.5 | 8 | 53.3 |
| Event 9 | 281 | 2 | 0.7 | 2 | 100.0 |
| Event 10 | 266 | 9 | 3.4 | 5 | 55.6 |
| Event 11 | 307 | 16 | 5.2 | 13 | 81.3 |
| Average | | | 2.7 | | 69.3 |
| Total | 3752 | 98 | | 66 | |

TABLE 12b
Genome Editing of ZM-ODP2-(266-668):AtCBF1a + Cyclin
Gene + Genome Editing + Cre Excision

| | | | | Edited | Edited |
|---|---|---|---|---|---|
| | Plant | Total | Edited | Plants | Plants |
| | Returning | Edited | Plants | with Cre | with Cre |
| Event ID | GEMC | Plants | (%) | Excision | Excision (%) |
| Event 1 | — | — | — | — | — |
| Event 2 | 10 | 8 | 80.0 | 4 | 40.0 |
| Event 3 | 10 | 8 | 80.0 | 5 | 50.0 |
| Event 4 | 11 | 10 | 90.9 | 6 | 54.5 |
| Event 5 | 14 | 11 | 78.6 | 8 | 57.1 |
| Event 6 | 8 | 8 | 100.0 | 6 | 75.0 |
| Event 7 | — | — | — | — | — |
| Event 8 | 15 | 13 | 86.7 | 7 | 46.7 |
| Event 9 | 2 | 2 | 100.0 | 2 | 100.0 |
| Event 10 | 8 | 5 | 62.5 | 4 | 50.0 |
| Event 11 | 14 | 13 | 92.9 | 11 | 78.6 |
| Average | | | 85.7 | | 61.3 |
| Total | 92 | 78 | | 53 | |

These data demonstrate it was possible to obtain a maternal embryo that was diploidized in vivo. Importantly, this outcome was achieved without performing in vitro tissue culture of an immature haploid embryo, and without a artificial chromosome doubling treatment, for example, a chemical chromosome doubling treatments, such as colchicine. Furthermore, it was possible to obtain such mature seed wherein Cre-mediated excision had occurred.

The method demonstrated a haploid induction rate (2.7%), wherein 85.7% of the progeny were gene edited and 61.3% were edited plants with Cre excision. Thus, this method demonstrates an improved level of productivity in comparison to the results shown in Example 9.

### Example 11: Methods of In Vivo Parthenogenic Haploid Induction and Genetic Chromosome Doubling Using AHL

The following experiment utilizes an expression cassette for conferring parthenogenic haploid induction and in vivo chromosome doubling. A first expression cassette expresses a full-length ZM-ODP2 peptide or variant thereof (WO2022087616A1), ZM-ODP2-(266-669) (SEQ ID NO: 2), or ZM-ODP2-(266-668):At-CBF1a fusion protein (SEQ ID NO: 15) that is operably linked to the PvEC1 promoter (SEQ ID NO: 6).

The second expression cassette expresses a polynucleotide encoding a AT-HOOK MOTIF CONTAINING NUCLEAR LOCALIZED (AHL) peptide, for example, an AHL family member as shown in Table 13. The AHL expression cassette is operably linked to a tissue-specific promoter such as “PvEC1 promoter” (SEQ ID NO: 6) and used to transform cells as described in Example 2.

TABLE 13
AHL gene family members

| | DNA SEQ | Protein SEQ |
|---|---|---|
| AHL family member | ID NO | ID NO |
| At-AHL15 | 53 | 54 |
| At-AHL19 | 55 | 56 |
| At-AHL20 | 57 | 58 |
| Zm00001d028577 | 59 | 60 |
| Zm00001d020768 | 61 | 62 |
| ZM00001d002495 | 63 | 64 |
| ZM00001d001784 | 65 | 66 |
| ZM00001d049521 | 67 | 68 |

It is expected that when the combined activities of a BBM/ZM-ODP2 peptide and a AHL family member are provided to an unfertilized egg cell, a combination of parthenogenic haploid induction and in vivo haploid genome doubling will be observed. It is expected that ectopic expression of an AHL family member will increase chromatin decondensation within the unfertilized egg cell, thereby promoting elevated levels of cell fate reprogramming, particularly for genes regulated by BBM/ZM-ODP transcription factor activity, and that the global chromatin decondensation induced by the ectopic AHL activity within the unfertilized egg cell can contribute to endomitosis and promote in vivo haploid genome doubling.

### Example 12: Methods of In Vivo Parthenogenic Haploid Induction and Genetic Chromosome Doubling Using ZM-ODP2-(266-668)-Regulatory Domain Fusions

In an alternative method to investigate parthenogenic haploid induction and in vivo genetic chromosome doubling in an unfertilized egg cell, a translational fusion protein comprising a heterologous, synthetic transcription factor is used.

This method uses a polynucleotide containing ZM-ODP2 (TR5)-V1 (SEQ ID NO:8) encoding the ZM-ODP2-(266-668) polypeptide (SEQ ID NO: 9) fused with a linker sequence (DNA SEQ ID NO: 10; Protein SEQ ID NO: 11) which in turn is fused to a maize optimized DNA sequence encoding a regulatory domain as detailed in Table 14. Together the coding sequence of the fusion protein, for example ZM-ODP2-(266-668):AT-CBF3I, is operably linked to the “PvEC1 promoter” (SEQ ID NO: 6).

Using the methods described in Examples 2 and 3, T0 transgenic plants hemizygous for the ZM-ODP2-(266-668)-Regulatory Domain Fusion are obtained from a population of transformed immature diploid embryos. The T0 transgenic plants are used as female parent plants and the donor ears fertilized with pollen provided by a pollen donor.

It is expected that when the ZM-ODP2-(266-668)-Regulatory Domain Fusion are provided to an unfertilized egg cell, a combination of parthenogenic haploid induction and in vivo haploid genome doubling will be observed.

TABLE 14
Fusion Protein Domains

| | DNA SEQ | PROTEIN SEQ |
|---|---|---|
| Domain | ID NO | ID NO |
| GNAT1 | 26 | 32 |
| GNAT2 | 27 | 33 |
| HAT1 | 28 | 34 |
| HAT2 | 29 | 35 |
| JMJ | 30 | 36 |
| SV40:VP64 | 31 | 37 |
| AT-CBF3I | 69 | 70 |
| HH-VP16 | 71 | 72 |
| VP16 | 73 | 74 |

### Example 13: Methods of In Vivo Parthenogenic Haploid Induction, Genetic Chromosome Doubling, and Genome Modification Using ZM-ODP2-(266-668)-Regulatory Domain Fusions

This experiment uses a non-haploid inducer line that is an F1 hybrid obtained by cross-fertilization of two parental lines. Approximately 10 days after pollination, an immature, diploid F1 embryo is transformed using the methods described in Examples 2 and 3. Simultaneous genome modification, parthenogenic haploid induction, and in vivo chromosome doubling activities is performed using a plasmid containing a T-DNA capable of regenerating a F1 hybrid T0 plant with i.) a synthetic transcription factor coding sequence that encodes a fusion protein of ZM-ODP2-(266-668) and a regulatory domain as detailed in Table 14 that is operably linked to the PvEC1 promoter (SEQ ID NO: 6) for conferring parthenogenic haploid induction and in vivo chromosome doubling activities, ii.) a Cas9 gene editing expression cassette for creating a double strand break, iii) a dual guide RNA (gRNA) expression cassette for conferring targeted genome modification, iv.) a DsRED color marker expression cassette useful as a reporter gene product for detecting presence/absence of T-DNA integration, and v.) a maize optimized (MO) CRE recombinase expression cassette for excising the polynucleotide sequence intervening the two loxP sites within the plasmid sequence.

Post transformation, each regenerated plant that is a hemizygous T₀ (F1 hybrid) plant having one copy of a T-DNA is considered a unique event that is transplanted to soil for growth to maturity. The ears of each hemizygous T₀ plant are shoot-bagged before silk emergence to avoid any foreign pollen contamination. The silks of the ears on the plants of the female parent plants are pollinated with viable pollen grains. Such pollen grains are collected from the anthers of a male non-haploid inducer parent plant constitutively expressing a cyan fluorescent protein color marker (CFP).

1. A method of producing a doubled haploid plant, the method comprising:
providing a plant cell with a polynucleotide sequence encoding at least a truncated ZM-ODP2 polypeptide;
regenerating a T0 plant from the plant cell, wherein the T0 plant expresses the polynucleotide sequence encoding the truncated ZM-ODP2 polypeptide;
obtaining a donor ear from the T0 plant;
pollinating the donor ear with pollen from a pollen donor plant;
expressing the polynucleotide sequence encoding the truncated ZM-ODP2 polypeptide, wherein the truncated ZM-ODP2 polypeptide promotes chromosome doubling of a haploid embryo to produce a doubled haploid embryo;
selecting a doubled haploid embryo lacking the genome of the pollen donor plant; and
regenerating a doubled haploid plant from the doubled haploid embryo or a mature seed thereof.

2. The method of claim 1, wherein chromosome doubling is achieved without a chemical chromosome doubling agent.

3. The method of claim 1, further comprising providing a second genetic chromosome doubling agent to the plant cell, wherein the second genetic chromosome doubling agent comprises a polynucleotide sequence encoding a cyclin gene family member.

4. (canceled)

5. (canceled)

6. (canceled)

7. The method of claim 5, wherein the pollen donor plant comprises a paternal marker gene that is expressed in embryo tissue.

8. The method of claim 1, wherein the truncated ZM-ODP2 polypeptide comprises an amino acid sequence having at least 90% sequence identity to SEQ ID NO: 2.

9. The method of claim 1, wherein the truncated ZM-ODP2 polypeptide is part of a fusion protein that further comprises CBF1a, CBF3I, GNAT1, GNAT2, HAT1, HAT2, JMJ, VP16, or SV40:VP64.

10. The method of claim 9, wherein the fusion protein comprises the truncated ZM-ODP2 polypeptide and CBF1a, and wherein the fusion protein comprises an amino acid sequence having at least 95% sequence identity to SEQ ID NO: 15.

11. A method of producing a doubled haploid plant, the method comprising:
stimulating parthenogenic haploid induction and chromosome doubling by providing a haploid plant cell with a polynucleotide sequence encoding at least a truncated ZM-ODP2 polypeptide;
regenerating a T0 plant expressing the polynucleotide sequence encoding the truncated ZM-ODP2 polypeptide, wherein a haploid set of chromosomes is diploidized;
pollinating the T0 plant;
obtaining a doubled haploid embryo from the T0 plant; and
regenerating a doubled haploid plant from the doubled haploid embryo or a mature seed thereof.

12. The method of claim 11, wherein diploidization is achieved without a chemical chromosome doubling agent.

13. The method of claim 11, further comprising providing a second genetic chromosome doubling agent to the haploid plant cell, wherein the second genetic chromosome doubling agent comprises a polynucleotide sequence encoding a cyclin gene family member.

14. (canceled)

15. The method of claim 1, wherein pollinating the T0 plant comprises self-pollination.

16. The method of claim 1, wherein pollinating the T0 plant comprises pollinating the T0 plant with pollen.

17. The method of claim 11, wherein the truncated ZM-ODP2 polypeptide comprises an amino acid sequence having at least 90% identity to SEQ ID NO: 2.

18. The method of claim 11, wherein the truncated ZM-ODP2 polypeptide is part of a fusion protein that further comprises CBF1a, CBF3I, GNAT1, GNAT2, HAT1, HAT2, JMJ, or SV40:VP64.

19. The method of claim 18, wherein the fusion protein comprises the truncated ZM-ODP2 polypeptide and CBF1a, and wherein the fusion protein comprises an amino acid sequence having at least 95% sequence identity to SEQ ID NO: 15.

20. A method of producing a genome-edited, doubled haploid plant, the method comprising:
providing a plant cell with:
(i) a polynucleotide sequence encoding at least a truncated ZM-ODP2 polypeptide; and
(ii) a polynucleotide sequence encoding a genome-editing component;
regenerating a T0 plant from the plant cell, wherein the T0 plant expresses the polynucleotide sequence encoding at least the truncated ZM-ODP2 polypeptide and the polynucleotide sequence encoding the genome-editing component;
obtaining a donor ear from the T0 plant;
pollinating the donor ear with pollen from a pollen donor;
expressing the polynucleotide sequence encoding the truncated ZM-ODP2 polypeptide and the polynucleotide sequence encoding the genome-editing component, wherein the truncated ZM-ODP2 polypeptide promotes chromosome doubling of a haploid embryo to produce a doubled haploid embryo;
selecting a doubled haploid embryo lacking the genome of the pollen donor plant; and
regenerating a doubled haploid plant from the doubled haploid embryo or a mature seed thereof.

21. The method of claim 20, wherein chromosome doubling is achieved without a chemical chromosome doubling agent.

22. The method of claim 20, further comprising providing a second genetic chromosome doubling agent to the plant cell, wherein the second genetic chromosome doubling agent comprises a polynucleotide sequence encoding a cyclin gene family member.

23. (canceled)

24. (canceled)

25. (canceled)

26. The method of claim 20, wherein the pollen donor plant comprises a paternal marker gene that is expressed in embryo tissue.

27. The method of claim 20, wherein the truncated ZM-ODP2 polypeptide comprises an amino acid sequence having at least 90% identity to SEQ ID NO: 2.

28. The method of claim 20, wherein the truncated ZM-ODP2 polypeptide is part of a fusion protein that further comprises CBF1a, CBF3I, GNAT1, GNAT2, HAT1, HAT2, JMJ, or SV40:VP64.

29. The method of claim 28, wherein the fusion protein comprises the truncated ZM-ODP2 polypeptide and CBF1a, and wherein the fusion protein comprises an amino acid sequence having at least 95% sequence identity to SEQ ID NO: 15.

30. The method of claim 20, wherein the genome-editing component is a Cas9 nuclease or a Cas alpha nuclease, and the method further comprises providing the plant cell with a guide polynucleotide.

31. A method of producing a doubled haploid plant, the method comprising:
inducing somatic embryogenesis in a haploid embryo;
transforming the haploid embryo with a polynucleotide sequence encoding a truncated ZM-ODP2 polypeptide;
obtaining a somatic embryo or somatic embryogenic tissue expressing the polynucleotide sequence encoding the truncated ZM-ODP2 polypeptide;
culturing the somatic embryo or somatic embryogenic tissue to obtain a plantlet, wherein a haploid set of chromosomes is diploidized; and
regenerating a doubled haploid plant from the plantlet or a mature seed thereof.

32. The method of claim 31, wherein inducing somatic embryogenesis in the haploid embryo comprises transforming the haploid embryo with a morphogenic gene expression cassette comprising:
(i) a polynucleotide sequence encoding a WUS/WOX polypeptide, wherein the WUS/WOX polypeptide is selected from WUS1, WUS2, WUS3, WOX2A, WOX4, WOX5, and WOX9;
(ii) a polynucleotide sequence encoding a ZM-ODP2 polypeptide, wherein the ZM-ODP2 polypeptide is selected from BBM2, BMN2, BMN3, and ODP2; or
(iii) a combination of (i) and (ii).

33. The method of claim 31, wherein diploidization is achieved without a chemical chromosome doubling agent.

34. The method of claim 31, a wherein the truncated ZM-ODP2 polypeptide comprises an amino acid sequence having at least 90% sequence identity to SEQ ID NO: 2.

35. A method of producing a genome-edited doubled haploid plant, the method comprising:
inducing somatic embryogenesis in a haploid embryo;
transforming the haploid embryo with:
(i) a polynucleotide sequence encoding a truncated ZM-ODP2 polypeptide; and
(ii) a polynucleotide sequence encoding a genome-editing component;
obtaining a somatic embryo or somatic embryogenic tissue expressing the polynucleotide sequence encoding the truncated ZM-ODP2 polypeptide and the polynucleotide sequence encoding the genome-editing component;
culturing the somatic embryo or somatic embryogenic tissue to obtain a plantlet, wherein a haploid set of chromosomes is diploidized; and
regenerating a doubled haploid plant from the plantlet or a mature seed thereof.

36. The method of claim 35, wherein inducing somatic embryogenesis in the haploid embryo comprises transforming the haploid embryo with a morphogenic gene expression cassette comprising:
(i) a polynucleotide sequence encoding a WUS/WOX polypeptide, wherein the WUS/WOX polypeptide is selected from WUS1, WUS2, WUS3, WOX2A, WOX4, WOX5, and WOX9;
(ii) a polynucleotide sequence encoding a ZM-ODP2 polypeptide, wherein the ZM-ODP2 polypeptide is selected from BBM2, BMN2, BMN3, and ODP2; or
(iii) a combination of (i) and (ii).

37. The method of claim 35, wherein diploidization is achieved without a chemical chromosome doubling agent.

38. The method of claim 35, wherein the truncated ZM-ODP2 polypeptide comprises an amino acid sequence having at least 90% identity to SEQ ID NO: 2.

39. The method of claim 35, wherein the genome-editing component is a Cas9 nuclease or a Cas alpha nuclease, and the method further comprises providing the haploid embryo with a guide polynucleotide.
